(19) United States
(12) Patent Application Publication
(10) Pub. No.: US 2011/0190679 A1
(43) Pub. Date: Aug. 4, 2011

(54) EXTRACORPOREAL CELL-BASED THERAPEUTIC DEVICE AND DELIVERY SYSTEM

(75) Inventors: H. David Humes, Ann Arbor, MI (US); Deborah Buffington, Ann Arbor, MI (US); Gretchen Hageman, Ann Arbor, MI (US)

(73) Assignee: Innovative BioTherapies, Inc., Ann Arbor, MI (US)

(21) Appl. No.: 13/027,481

(22) Filed: Feb. 15, 2011

Related U.S. Application Data
(63) Continuation of application No. PCT/US2009/053516, filed on Aug. 12, 2009.
(60) Provisional application No. 61/189,119, filed on Aug. 15, 2008.

(51) Int. Cl. A61M 1/36; A61M 1/28; C12N 5/00

(52) U.S. Cl. 604/4.01; 604/28; 422/44; 435/402

(57) ABSTRACT

Extracorporeal cell-based therapeutic devices and delivery systems are disclosed which provide a method for therapeutic delivery of biologically active molecules produced by living cells in response to a dynamic physiologic environment. One embodiment includes long hollow fibers in which a layer of cells are grown within the intraluminal volume or within a double hollow-filled chamber. Another embodiment includes a wafer or a series of wafers providing a substrate onto which cells are grown. The wafer(s) are inserted into a device. A device may deliver a pre-selected molecule, for example, a hormone, into a mammal's systemic circulation and/or may deliver a member of different cell products. The device is adapted to secure viable cells which produce and secrete the pre-selected molecule into blood or fluid. The invention also provides a minimally invasive method for percutaneously introducing into a preselected blood vessel or body cavity the device of the invention.

### CROSS-REFERENCE TO RELATED APPLICATIONS

This application is a continuation of the International Patent Application PCT/US09/53516, filed Aug. 12, 2009, which claims priority to and the benefit of U.S. Provisional Patent Application No. 61/189,119, filed Aug. 15, 2008, the entire disclosures of each of which are incorporated herein by reference.

### STATEMENT REGARDING FEDERALLY SPONSORED RESEARCH OR DEVELOPMENT

This invention was made with government support under grant number W81XWH-05-2-0010, and modification numbers P00004 and P00005, awarded by the U.S. Army Medical Research and Materiel Command, and grant number 2R44DK074289-03, awarded by the National Institute of Diabetes and Digestive and Kidney Diseases. The government has certain rights in the invention.

### FIELD OF THE INVENTION

The present invention relates to an extracorporeal therapeutic device for delivering therapeutic molecules into a body. More particularly, this invention relates to an extracorporeal therapeutic device containing viable cells. One way that the molecules can be delivered into a body is through blood circulation or other bodily fluids. When the device is introduced into the blood stream outside the body, the cells produce and secrete the therapeutic molecule into blood or fluid circulating past the device.

### BACKGROUND OF THE INVENTION

Drug delivery devices useful for introducing therapeutic molecules into a mammal have been the subject of considerable research. In one aspect, the research has focused on the development of devices that deliver molecules produced from cellular metabolism. Efforts have also focused on producing an implantable cell based delivery system that can remain in a patient for an extended period of time.

An implantable device for delivering a pre-selected molecule, for example, a hormone, into a mammal's systemic circulation is described in U.S. Pat. No. 6,716,208, the entire contents of which is incorporated by reference. The device described comprises an implantable blood permeable element that can be anchored to an inner wall of an intact blood vessel and a capsule that is held in place within the blood vessel by the anchored blood permeable element. The capsule encloses viable cells which produce and secrete the preselected molecule into blood passing the capsule. The patent also describes a method for percutaneously introducing the device into a preselected blood vessel.

Intracorporeal cell based delivery devices must be sized to fit within a body, typically a body lumen (such as a blood vessel) and, accordingly, have certain size limitations because of the reduced-size requirements. Further, when an intracorporeal cell based delivery system is designed, the anchors used to attach the device to the body, e.g., blood vessel, must be configured to secure the device without introducing trauma to the body. Finally, implantable devices can be difficult to retrieve, especially if they are left within the body for an extended period of time.

In vitro experiments with a larger extracorporeal device utilizing porcine or human proximal tubule progenitor cells have shown differentiated transport and metabolic functions of the renal tubule assist device (RAD).[references 4-6] The bioartificial kidney (BAK) set-up consists of a filtration unit (a conventional synthetic hemofiltration cartridge) followed in series by the tubule (RAD) unit. The tubule unit is able to maintain viability because oxygen along with metabolic substrates and growth substances are delivered to the tubule cells from both intraluminal ultrafiltrate and blood in the extracapillary space Immunoprotection of the cells is achieved due to the impenetrability of immunoglobulins and immunocompetent cells across the hollow fibers. Rejection of non-autologous cells does not occur.

Pre-clinical studies in large animals have demonstrated that the BAK successfully replaced filtration, transport, metabolic, and endocrinologic functions of the kidney in acutely uremic dogs.[5] Further pre-clinical experiments in acutely uremic dogs have also evaluated the influence of the RAD under stress states. Acutely nephrectomized animals were challenged with infusions of endotoxin (lipopolysaccharide) intravenously or with intraperitoneal administration of doses of viable E. Coli before treatment with either cell or sham control RADs in a BAK.[references 7,8] In these experiments, cell RADs provided metabolic renal replacement and resulted in higher anti-inflammatory plasma levels, better hemodynamic stability, and, in the E. Coli sepsis model, longer survival times compared to sham controls. To further evaluate the role of the BAK in septic shock, a swine model with normal kidney function was given large doses of E. Coli intraperitoneally.[reference 9] All animals developed acute tubular necrosis (ATN) with oligo/anuria within 2-4 hours following administration, and RAD treatment resulted in better cardiovascular performance, lower plasma levels of the pro-inflammatory cytokines, and longer survival times compared to sham controls.

These supportive pre-clinical experiments were the basis for testing human cell RADs in Phase I/II and Phase II clinical trials in intensive care unit (ICU) patients with acute renal failure (ARF) and multi-organ failure (MOF). A favorable Phase I/II safety trial [reference 10] led to an FDA-approved, randomized, controlled, open-label Phase II investigation at 12 clinical sites to determine whether this cell therapy approach alters patient mortality. This Phase II study involved 58 patients, of whom 40 were randomized to RAD therapy and 18 made up a control group with comparable demographics and severity of illness. The early results have been as compelling as the Phase I/II results. Renal cell therapy improved the 28-day mortality rate from 61% in the conventional hemofiltration-treated control group to 34% in the RAD-treated group.[references 11,12] This survival impact continued through the 90- and 180-day follow-up periods (p<0.04), with the Cox proportional hazard ratio indicating that the risk of death was 50% of that observed in the conventional continuous renal replacement therapy group. This survival advantage with renal cell therapy was observed for various etiologies of ARF and regardless of organ failure number (1 to 5+) or the presence of sepsis. Subset analysis of patients with concomitant severe sepsis or septic shock demonstrated an incidence of sepsis of 73% and 67% in the cell therapy and conventional therapy groups, respectively. RAD therapy was associated with a mortality rate of 34% in patients with sepsis, compared to 67% in the conventional treatment group. Thus, these clinical results suggest a major effect on survival rates in these desperately ill patients. The clinical use of renal tubule cell therapy for patients with severe sepsis will not require this complex two-cartridge system with two extracorporeal pump systems, since most are not in ARF.

The present invention provides an extracorporeal cell based delivery system that is designed to address aspects of an intracorporeal cell based delivery system. Further, the present invention provides an extracorporeal device designed to introduce therapeutic agents into a mammal that secretes a pre-selected molecule or a combination of cell products directly into the blood stream or into a body fluid or body cavity and addresses the challenges of the prior art. The invention will be more clearly understood from the description, which follows.

Further, a miniaturized cell therapy device will not require extensive extracorporeal blood pump systems. In addition, a miniaturized device that could be stored at the clinical site for immediate use is required to succeed as a commercial product. The current RAD is stored at a central manufacturing facility at 37° C. and must be shipped at 37° C. to the clinical site, delaying treatment and adding to the cost of therapy. Development of a cell device that can be cryopreserved and stored at clinical sites can help safely bring the device to market.

### SUMMARY OF THE INVENTION

The present invention provides an extracorporeal therapeutic device for delivery of a pre-selected molecule or cell products into a mammal, for example, into the circulatory system or body fluids of a mammal. An embodiment of the invention enables molecules to be introduced into the circulatory system or a body cavity without invasive surgical procedures. Once the device is deployed, it delivers the molecule directly into the blood stream or body fluid. In addition, the device of the invention is adapted to produce and thereafter secrete the pre-selected molecule or cell product into the blood stream or body fluid over a determined period of time. The extracorporeal device and method provide an easy and reproducible system for delivering therapeutically effective amounts of a gene product, for example, a hormone, growth factor, anti-coagulant, immunomodulator, or the like, directly into the blood stream or body fluid of the recipient without the disadvantages of an invasive implantation procedure.

An extracorporeal device that administers a preselected molecule(s) into the mammal over a predetermined period presents advantages over the prior art. An extracorporeal device has the advantage of being easily taken out of the circulation system compared to the efforts required to remove an implanted device. Accordingly, the present invention provides an extracorporeal device for delivering, over a determined period of time, a preselected molecule or cell products into the systemic circulation of a mammal. In another aspect, the present invention provides a method for non-surgically introducing the device into blood circulation of a mammal that is capable of delivering the preselected molecule or cell products into systemic circulation.

In one embodiment, the device includes a capsule that contains viable cells which produce and secrete the preselected molecule into the blood stream. The device may include an anchoring element, which anchors the device to an inner surface of a tube that circulates blood extracorporeally.

The term “extracorporeal circuit” as used in this specification embraces any tube or conduit outside the body that may be connected to the circulatory system or body fluid compartment in a mammal and provides for the flow of blood or fluid through the tube or conduit by natural (e.g., heart) or artificial (e.g., mechanical pump) circulation. An extracorporeal device of the present invention is configured to be disposed in an extracorporeal circuit.

The term “anchoring element” as used in this specification embraces any structure that may be inserted into the lumen of an extracorporeal circulatory system blood tube or conduit and that, once inserted, may be anchored, for example, by hooks, barbs, or stents, to an inner surface of the tube or conduit. In an exemplary embodiment, the anchoring element may be a blood clot filter-type structure. A variety of blood clot anti-migration filters useful in the practice of the invention are known in the art. The currently preferred anchoring element is an anti-migration filter known as a “Greenfield® vena cava filter”. Useful Greenfield® vena cava filters are described in detail in U.S. Pat. Nos. 4,817,600 and 5,059,205, the entire disclosures of which are incorporated by reference.

The term “capsule” as used in this specification embraces any hollow structure dimensioned to fit within the lumen of a tube or conduit used in an extracorporeal circuit and does not occlude or prevent blood or fluid flow. In one embodiment, the capsule is held in place within the extracorporeal blood circuit by anchoring element(s). For example, the capsule may be retained upstream of the anchoring element, alternatively, the anchoring element may be located downstream of the anchoring element and retained in place by an attachment, for example, a hook or tether, extending from the anchoring element to the capsule. In addition, the capsule may be conical or wedge-like in shape to decrease the turbulence of blood flowing past the capsule. In a preferred embodiment, the capsule is formed from a material that can filter particles such that particles (including cells) below a certain size can pass through and particles above a certain size are prevented from passing through. The filter forms an ultrafiltrate from the blood to minimize the entry of proteins greater than 100,000 molecular weight so that immunoglobulins can be excluded from the bathing media around the cells, especially for nonautologous cells not to activate an immunologic response.

The capsule may include either a single hollow fiber or a bundle of hollow fibers made from a semi-permeable membrane. The semi-permeable membrane filter preferably has pores of a size sufficient to permit the diffusion of a preselected molecule or cell products therethrough but yet small enough to exclude the passage of cells therethrough. The pores preferably are designed to permit the preselected molecule produced by the cells to diffuse directly into the blood stream passing the hollow fiber while preventing the cells from migrating out of the hollow fiber and into the systemic circulation.

A variety of polymers are useful in producing the biocompatible semi-permeable membrane of the present invention. They include, but are not limited to polyalginate, polyvinylchloride, polyvinylidene fluoride, polyurethane isocyanate, cellulose acetate, cellulose diacetate, cellulose triacetate, cellulose nitrate, polysulfone, polystyrene, polyurethane, polyvinyl alcohol, polyacrylonitrile, polyamide, polymethylmethacrylate, polytetafluoroethylene, and polyethylene oxide. In addition, useful semi-permeable membranes may be produced from a combination of such polymers.

In an embodiment, the viable cells enclosed within the semi-permeable hollow fiber(s) of the capsule, preferably are eukaryotic cells, and most preferably are mammalian cells. Although the device described herein may comprise cells which naturally produce and secrete the preselected molecule or other cell products, it is contemplated that genetically engineered cells, i.e., cells transfected with, and capable of expressing a nucleic acid encoding the pre-selected molecule, may likewise be used in the practice of the invention.

In another embodiment, the preselected molecule can be a protein, and most preferably is a hormone, for example, erythropoietin or insulin. It is contemplated, however, that the device may be used to deliver into the systemic circulation any molecule that can be produced and secreted from a viable cell. Although single cell types that produce and secrete a single preselected molecule may be used in the invention, it is understood that cells belonging to a particular cell type that produce and secrete a plurality of preselected molecules likewise may be used in the practice of the present invention. Similarly, it is contemplated that a plurality of cell types, wherein cells belonging to each cell type produce and secrete different preselected molecules, may be combined in a capsule thereby to produce a device that delivers a desirable combination of preselected molecules into the circulation.

Preferred embodiments of the device include three configurations. Each preferred configuration isolates the therapeutic cells to minimize the immune response. In a first configuration, a device consists of a cartridge, a cell bearing unit which may be in the form of tubes attached to the cartridge and an anchoring system. The therapeutic cells are disposed within the tubes and the cells are isolated by the size of the pores in the tube. In a second configuration, the cell bearing unit is in the form of disks that are disposed in the cartridge. The therapeutic cells on the disks are protected from immunologic rejection by isolating the disks in the cartridge and providing pores in the cartridge that prevents the cells from being exposed to undesirable elements while allowing free physiologic exchange for the cells within the extracorporeal blood or fluid stream. A third configuration is a combination of the configurations described above. Other configurations are possible.

The devices utilizing cells disposed in hollow fibers and/or cells disposed upon a substrate, such as disks, also can be included in extracorporeal circuits utilizing pumps and fluid separation devices (e.g., hemofilters, dialyzers, and the like) which can provide the devices with fluids for treatment, such as ultrafiltrate derived from blood or peritoneal fluid or peritoneal fluid itself, such that cells are protected from molecules in the body fluids having the ability to mount an immune response. Such extracorporeal circuits can be used in various applications, including, for example, chronic applications and acute applications. In chronic applications, cells can be isolated from a biopsy taken from the subject to be treated and these cells can be expanded prior to use. Because such cells are derived from a host subject, an immunoprotective barrier, such as a filter, may not be needed. In acute applications, the filters may provide immunoprotection by limiting the immune-stimulating molecules from passing through and contacting the cells. Additionally, the animal is protected from the cells which could elicit an immune response by contact of the molecules produced by the cells. Any cells can be used in embodiments according to the invention, such as eukaryotic cells, such as mammalian cells, and such as renal cells.

Thus, in one aspect of the invention, an extracorporeal therapeutic system includes a housing defining an interior space, a frozen substrate having a trabecular structure disposed within the housing, and at least one cell disposed on the substrate. This aspect or any of the following aspects can have any of the following features. The housing can include an inlet for receiving a fluid and an outlet for releasing a processed fluid. At least one flow separator can be disposed between the inlet and the substrate. The substrate can be initially separate from the housing, and/or can be coated with a biomatrix material such as collagen IV.

In another aspect of the invention, an extracorporeal therapeutic system includes a housing defining an interior space, a substrate including a carbon material coated with niobium disposed within the housing, and at least one cell disposed on the substrate. This aspect or any of the following aspects can have any of the following features. The substrate can have a trabecular structure, and the substrate can be initially separate from the housing. The substrate can be coated with collagen IV. The substrate can also be coated with any three-dimensional biomatrix material that protects the integrity and durability of the carbon material, and the biomatrix material can promote or increase cell expansion, attachment, and/or viability. The housing can include an inlet for receiving a fluid and an outlet for releasing a processed fluid. At least one scaffold to retain the substrate can be disposed within the housing, and at least one flow separator can be disposed between the inlet and the substrate. At least one baffle can also be disposed between the inlet and the substrate. The substrate can be frozen.

In another aspect of the invention, an extracorporeal therapeutic system includes a housing, at least one hollow fiber associated with the housing, a biomatrix material disposed within the at least one hollow fiber, and at least one cell disposed within the biomatrix material. This aspect or any of the following aspects can have any of the following features. The cell can be disposed upon a particle. The biomatrix material can be selected from, but is not limited to, alginate solution, gelled alginate solution, or physiologic buffer, and the biomatrix material can also be selected from nutragen or matrigel. Other examples of biomatrix material can include collagen I or any three-dimensional forming or formed biocompatible matrix. The hollow fiber can be disposed within the housing, and the housing can include an inlet and an outlet.

In another aspect of the invention, a substrate for maintaining cells includes a carbon material coated with niobium and with collagen IV and has a trabecular structure. This aspect or any of the following aspects can have the following features. The substrate can also include a cell that has the potential to provide therapeutic value, such as a eukaryotic cell, a mammalian cell, and/or a renal cell. The substrate can be frozen.

In another aspect of the invention, a method for loading an extracorporeal therapeutic system includes thawing a cryopreserved cell and loading the cell into the system. This aspect or any of the following aspects can have any of the following features. The system can include a housing and at least one hollow fiber associated with the housing, and the cryopreserved cell is thawed, mixed with a biomatrix material, and loaded into the hollow fiber. The system also or alternatively can include the cryopreserved cell disposed on a substrate, which can be loaded into the housing. The substrate can include a carbon material coated with niobium and, optionally, collagen IV.

In another aspect of the invention, a method for extracorporeal therapy includes connecting an extracorporeal therapeutic system to the peritoneum of a mammal, so that a peritoneal fluid of the mammal circulates through the system. This aspect or any of the following aspects can have any of the following features. The system can include a housing and at least one hollow fiber associated with the housing, and the system also can include a biomatrix material disposed within the hollow fiber and at least one cell disposed within the biomatrix material. The system also or alternatively can include a housing and a substrate including a carbon material coated with niobium and, optionally, collagen IV, and at least one cell can be disposed on the substrate. The system also or alternatively can include a housing and a frozen substrate having a trabeculated structure. At least one cell can be disposed on the substrate. The housing can also include an inlet for receiving fluid and an outlet for releasing a processed fluid.

In another aspect of the invention, an extracorporeal cell based therapeutic device includes (a) an anchor system which can be capable of attaching the device to an extracorporeal tube, which when attached to the inner wall of the tube permits blood in the tube to pass therethrough; and (b) a capsule including a plurality of pores and having viable cells disposed therein, so that the capsule, when introduced into the tube, can be retained within the tube by the anchor system and the pores permit nutrients to enter the capsule to maintain viability of the cells disposed therein. This aspect or any of the following aspects can have any of the following features. The capsule can be defined by a semi-permeable membrane, and the semi-permeable membrane can include a material selected from polyvinylidene fluoride, polyvinylchloride, polyurethane, polyalginate, polystyrene, polyurethane, polyvinyl alcohol, polyacrylonitrile, polyamide, polymethylmethacrylate, polyethylene oxide, polytetrafluorethylene, isocyanate, cellulose acetate, cellulose diacetate, cellulose triacetate, cellulose nitrate, polysulfone, and mixtures thereof. The viable cells can be disposed on a plurality of filaments, and the filaments can be metallic. The pores can be dimensioned to prevent passage of antibodies therethrough, and the cells can be disposed on at least one disk. The cells can be eukaryotic cells, such as mammalian cells. The capsule can be adapted to be separated from the tube by attaching and detaching the anchor system from the tube. The capsule can include at least one hollow fiber. The pores can permit solutes less than 150 kD to pass therethrough, and the device can be configured to provide a therapeutically significant amount of a molecule with or without using an artificial blood pump.

In another aspect of the invention, an extracorporeal cell based therapeutic device includes (a) an attachment system anchorable to an inner wall of an extracorporeal tube adapted to provide a conduit for blood, which when anchored to the inner wall of a tube permits blood to circulate; and (b) a capsule defining a plurality of pores and having viable cells disposed therein, so that the capsule can be retained within the tube by the attachment system and the pores permit nutrients to enter the capsule to maintain viability of the cells disposed therein. This aspect or any of the following aspects can have any of the following features. The pores can be dimensioned to exclude an agent in the blood which is detrimental to cell viability. The anchor can be metallic. The capsule when introduced into the tube can be retained upstream of the attachment system, and the capsule can include at least one disk. The cells can also be genetically engineered cells.

In another aspect of the invention, a method of introducing an extracorporeal cell-based therapeutic device into the circulatory system of a mammal includes the steps of: (a) anchoring an attachment system to an inner wall of an extracorporeal tube adapted to provide a conduit for bodily fluid, which when anchored to the inner wall of a tube permits the bodily fluid to circulate; and (b) inserting a capsule defining a plurality of pores and having viable cells disposed therein, so that the capsule can retained within the tube by the attachment system and the pores permit nutrients to enter the capsule to maintain viability of the cells disposed therein. This aspect or any of the following aspects can have any of the following features. In step (a), the attachment system can be introduced into the tube via a catheter, and in step (b), the capsule can be introduced into the tube via a catheter. The device can be used to deliver a therapeutic amount of a molecule in the conduit with or without the use of an artificial body fluid pump. The conduit can be attached to the peritoneum and the peritoneal fluid passes through the conduit.

In another aspect of the invention, an extracorporeal cell based therapeutic device includes (a) an attachment system anchorable to an inner wall of an extracorporeal tube adapted to provide a conduit for a bodily fluid, which when anchored to the inner wall of a tube permits the bodily fluid to circulate; and (b) a capsule defining a plurality of pores and having viable cells disposed therein, so that the capsule, when introduced into the tube, can be retained within the tube by the attachment system and the pores permit nutrients to enter the capsule to maintain viability of the cells disposed therein. This aspect can have any of the following features. The tube can be adapted to be connected to the peritoneum and the bodily fluid includes peritoneal fluid, and the tube can be adapted to be connected to blood vessels and the bodily fluid includes blood. The attachment system can be separable from the tube.

### BRIEF DESCRIPTION OF THE DRAWINGS

The foregoing and other objects, aspects, features, and advantages of the invention will become more apparent and may be better understood by referring to the following description taken in conjunction with the accompanying drawings, in which:

FIG. 1 is a perspective side view of a device according to an embodiment of the present invention;

FIG. 2 is a perspective side view of a device according to another embodiment of the present invention;

FIG. 3 is a detail cross section of the downstream end of the device illustrated in FIG. 2;

FIG. 4 is a cross section of the device illustrated in FIG. 2 and shows the flow of fluid through the device;

FIG. 5 is a perspective view of another embodiment of the invention;

FIG. 6 is a schematic cross section illustrating the embodiment of FIG. 5;

FIG. 7 is a detail view of an ultrafiltrate tube illustrated in FIGS. 5 and 6;

FIG. 8 is a schematic view of an extracorporeal blood circuit that can be used with embodiments of the present invention;

FIGS. 9-12 are graphs representing testing of embodiments of the present invention;

FIG. 13 is a perspective side view of another embodiment of the present invention and certain components of that embodiment;

FIG. 14 is a perspective side view of the device illustrated in FIG. 13;

FIG. 15 is a perspective view of another embodiment of the present invention;

FIG. 16 is an exploded view of the embodiment illustrated in FIG. 15;

FIG. 17A is a schematic view of a hollow fiber extracorporeal treatment device according to the invention and FIG. 17B is a schematic depicting cell seeding of the embodiment;

FIGS. 18A and B are schematic views of two extracorporeal blood circuits that can be used with an embodiment of the present invention, such as those shown in FIGS. 13-16;

FIG. 19 is a schematic view of an extracorporeal peritoneal fluid circuit that can be used with an embodiment of the present invention, such as those shown in FIGS. 13-16;

FIG. 20 is schematic view of another extracorporeal peritoneal fluid circuit that can be used with an embodiment of the present invention, such as those shown in FIGS. 13-16;

FIG. 21 is a schematic view of another extracorporeal peritoneal fluid circuit that can be used with an embodiment of the present invention, such as those shown in FIGS. 13-16;

FIG. 22 is a schematic of an extracorporeal blood circuit that can be used with an embodiment of the present invention, such as that shown in FIGS. 17A and B;

FIG. 23 is a graph of the effect of renal assist device intervention on mean arterial pressure in a septic pig;

FIG. 24 is a graph of the effect of renal assist device intervention on cardiac output in a septic pig;

FIG. 25 is a graph of the effect of renal assist device intervention on stroke volume in a septic pig;

FIG. 26 is a graph of the effect of renal assist device intervention on hematocrit in a septic pig;

FIG. 27 is a graph of the effect of renal assist device intervention on the survival rate of a septic pig;

FIG. 28 is a graph of the number of primary porcine renal epithelial cells (PPRECs) on collagen-IV coated disks over time;

FIGS. 29A-D show cell growth on carbon disks;

FIG. 30 is a graph of the average oxygen consumption for pre- and post-cryopreserved PPREC cells;

FIG. 31 is a graph of glucose consumption rates for pre- and post-cryopreserved PPREC cells;

FIG. 32 is a graph of lactate production for pre- and post-cryopreserved PPREC cells;

FIG. 33 is a graph of the survival times of a septic pig treated with a renal assist device, a sham renal assist device, and embodiments of the present invention (the device of FIG. 13 used in the circuits of FIGS. 18A and B);

FIG. 34 is a graph of non-stimulated and Lipopolysaccharide (LPS)-stimulated normal leukocytes under standard media and PPREC-conditioned media exposure;

FIG. 35 is a graph of an assessment of leukocyte activation in multiple cell type populations under normal conditions, after 5 hours of sepsis and after maximal stimulations by phorbol myristate acetate (PMA);

FIG. 36 is a graph of a Kaplan-Meier survival curve through 180 days comparing continuous venovenous hemofiltration (CVVH) therapy and CVVH therapy plus RAD therapy;

FIG. 37 is a graph of lamb-renal epithelial cell (LREC) active disk oxygen consumption rates versus fixed disk oxygen consumption rates based on in vitro testing of the embodiment illustrated in FIG. 13;

FIG. 38 is a graph of the difference in glucose consumption rates of LRECs after a freeze thaw process based on in vitro testing of the embodiment illustrated in FIG. 13;

FIG. 39 is a graph of the difference in oxygen consumption rates of LRECs after a freeze thaw process based on in vitro testing of the embodiment illustrated in FIG. 13;

FIG. 40 is a graph of the LREC oxygen consumption rates determined by ex vivo testing using the circuits illustrated in FIGS. 19-20;

FIG. 41 is a graph of the oxygen consumption rates determined by ex vivo testing using the circuits illustrated in FIGS. 19-20;

FIG. 42 is a graph of the pH change in peritoneal dialysate (PD) solution and serum, as assessed via arterial blood gas analysis, after recycling commences in testing using the circuits illustrated in FIGS. 19-20;

FIG. 43 is a graph of the bicarbonate change in PD solution and serum, as assessed via arterial blood gas analysis, after recycling commences in testing using the circuits illustrated in FIGS. 19-20;

FIG. 44 is a graph of the potassium change in PD solution and serum, as assessed via arterial blood gas analysis, after recycling commences in testing using the circuits illustrated in FIGS. 19-20;

FIG. 45 is a graph of the calcium change in PD solution and serum, as assessed via an Abbott Point-of-Care i-STAT analyzer, after recycling commences in testing of the circuits illustrated in FIGS. 19-20;

FIG. 46 is a graph of lactate production by live cells of the embodiment illustrated in FIGS. 15 and 16;

FIG. 47 is a graph of glucose consumption pre- and post-cryopreservation during testing of the embodiment illustrated in FIGS. 15 and 16;

FIG. 48 is an image of HEP-G2 cells grown to near tissue density in an embodiment of the present invention;

FIG. 49 is a graph of the growth curve of two flasks with PPREC-seeded microcarriers;

FIG. 50 is a graph of lactate production of PPREC-seeded microcarriers;

FIG. 51 is a graph of oxygen consumption of PPREC-seeded microcarriers;

FIG. 52 shows histology images of hollow fiber loaded PPREC-seeded microcarriers taken after 24 hours in culture. The image on the left is a 5× magnification of microcarriers loaded in a 500 nm inner diameter fiber. The image on the right is at 10× magnification;

FIGS. 53A-D show images of DAPI staining of PPRECs on gelatin microcarriers at 5× magnification. FIG. 53A shows an image of microcarriers pre-freeze. FIG. 53B shows an image of microcarriers post-thaw that were frozen in Cryostor™ CS5 cryopreservative medium. FIG. 53C shows an image of microcarriers post-thaw frozen in 5% DMSO. FIG. 53D shows an image of microcarriers post-thaw frozen in 10% DMSO; and

FIG. 54 is a graph of a comparison of IL-8 secretion rates on baseline and LPS-stimulated conditions in PPREC in two-dimensional culture.

FIG. 55 is a schematic view of another extracorporeal peritoneal fluid circuit that can be used with an embodiment of the present invention, such as those shown in FIGS. 13-16.

FIG. 56 is a graph of CD11b mean fluorescent intensity (MFI) during the course of human renal epithelial cell (HREC)-seeded BRECS-d cellular therapy in uremic sheep. MFI was measured at the following time points: (1) PRESURD1, which was 1 day prior to sheep nephrectomy; (2) PREBRD0, which was the time point just prior to BRECS-d therapy when the sheep were at their most uremic state; (3) BRD1 to BRD5, which corresponded to 1-5 days of BRECS-d therapy, respectively; (4) BR FINAL, which was the final day of BRECS-d therapy, in this case day 6, when the BRECS-d unit was removed; (5) POSTBRD1, which was 24 hours following BRECS-d removal; and (6) FINAL, which was the last time point of this study, occurring at 48 hours following BRECS-d removal. MR levels as shown are average values calculated based on the number of animals that remained in the study at the various time points.

FIG. 57 is a graph of the average oxygen consumption for LREC-seeded BRECS-d units and HREC-seeded BRECS-d units during the course of cellular therapy in normal and uremic sheep, respectively. The BRECS-d units were maintained for up to 13 days of therapy in normal sheep and up to 6 days of therapy in uremic sheep. Three point moving averages (value at the designated time point averaged with the 2 successive preceding time point values) were generated to display an accurate trendline. Oxygen consumption rates as shown are average values calculated based on the number of animals that remained in the study at the various time points.

FIGS. 58A-D are images of HRECs grown on collagen IV-coated carbon disks from BRECS-d units. Photographs are from three-dimensional microscopic fields of cut carbon disks to show en face labeling of surface antigens. FIG. 58A is an image of DAPI staining. FIG. 58B is an overlay immunohistochemistry (IHC) image of ZO-1 and AT-1 staining, indicative of renal epithelial cell tight junctions and centralized cilia, respectively. The insert depicts HRECs grown in monolayers on conventional cell culture plates, as opposed to three-dimensional disks, to better display these two differentiated phenotypes. FIG. 58C is an IHC image of CD 13, which is a brush border enzyme that facilitates Na⁺² dependent amino acid transport. FIG. 58D is an IHC image of γ-glutamyltranspeptidase (γGT), which is a brush border enzyme that facilitates glutathione metabolism. All primary antibodies were visualized with either the secondary antibody anti-rabbit IgG AlexaFluor 488 conjugate or anti-mouse IgG AlexaFluor 594 conjugate. FIG. 58A was taken with a 5× objective. FIGS. 58B-D were taken with a 10× objective.

FIG. 59 is a table showing commercially available antibodies that were used in the immunohistochemical analysis of HRECs.

FIG. 60 is a graph of the average cell number in PPREC-seeded BRECS-d units during a time course of 186 days in culture (n=4). Cell numbers were extrapolated from lactate production. Three point moving averages (value at the designated time point averaged with the 2 successive preceding time point values) were generated to display an accurate trendline. Corresponding cellular oxygen consumption rates in the BRECS-d units are also depicted.

FIG. 61 is a graph of glutathione (GSH) degradation rates in HREC-seeded BRECS-d units during a time course of 3 months in culture (n=4). GSH was measured during a 60 minute incubation period at t=0 and t=60 minutes.

FIG. 62 is graph showing temperature changes in four acellular BRECS-d units thawed over time using the thawing protocol described in Example 8. The four studies demonstrate internal temperature control without reaching temperatures above 39° C.

FIG. 63 is a graph of the average cell number in PPREC-seeded BRECS-d units before cryopreservation and post-thaw (n=5). BRECS-d units were frozen in CryoStor CS5 (Biolife Solutions, Inc.), which is a cryopreservation media containing 5% DMSO, and the units were thawed according to a 20 minute protocol described in Example 8. Points A and B identify the freeze and thaw time points, respectively. The average cell number per BRECS-d unit at various time points in culture is shown (Pre-Freeze and Post-Thaw represent before freezing and post thawing, respectively). Three point moving averages (value at the designated time point averaged with the 2 successive preceding time point values) were generated to display an accurate trendline.

FIG. 64 is a graph comparing the average cell number in a first group of PPREC-seeded BRECS-d units that were frozen and stored in liquid nitrogen vapor phase (−140° C.) (n=2) and a second group of units that were transferred from liquid nitrogen storage to −80° C. storage for 1 week and 1 month (n=2; one unit was stored at −80° C. for 1 week, and one unit was stored at −80° C. for 1 month) (“LqN Pre-FREEZE” and “LqN Post-THAW” are data from before freezing and storage in liquid nitrogen at −140° C. and after thawing, respectively; “−80 Pre-FREEZE” and “−80 Post-THAW” are data from before freezing at −140° C. and storage at −80° C. and after thawing, respectively). For the second group of BRECS-d units, the average cell number shown represents the average value calculated from a data set at each time point that included those units stored at −80° C. for 1 week and those units stored at −80° C. for 1 month. Points A and B denote cell numbers before freezing and after thawing, respectively, for units transferred from liquid nitrogen storage to −80° C. Points C and D show cell numbers before freezing and after thawing, respectively, for units stored only in liquid nitrogen vapor phase. Three point moving averages (value at the designated time point averaged with the 2 successive preceding time point values) were generated to display an accurate trendline. The x-axis reflects actual days in culture and does not include days in cryopreservation.

FIG. 65 is a graph showing temperature change as recorded by three thermocouples (TC1, TC3, TC4) placed inside a cryopreserved aceullular BRECS-d unit initially at the liquid nitrogen vapor phase (−140° C.). The unit was transferred to a Styrofoam cooler with dry ice, sealed and monitored over 36 hours to determine the length of time temperature could be maintained.

### DETAILED DESCRIPTION

The present invention provides an extracorporeal device for delivering molecules into a mammal, for example, the systemic circulation of a mammal. The device of the invention is adapted for introduction into an extracorporeal circuit, such as into an extracorporeal blood conduit or a peritoneal fluid circuit. After introduction into the extracorporeal circuit, such as the extracorporeal blood conduit, the device permits the pre-selected molecule(s) or cell product(s) to diffuse out of the device. In the case of the extracorporeal circuit, these pre-selected molecule(s) or cell product(s) diffuse out of the device and into the blood stream or body cavity of the recipient, which in certain aspects does so in response to blood parameters, for example, oxygen tension in the case of erythropoietin-producing cells. Also, the glucose concentrations in the bathing media around the insulin producing cells can stimulate the production of insulin.

The embodiments of the invention have at least some of the following advantages. Because the device is extracorporeal, there is less size constraint and the cell bearing units can be sized larger than if a device was implanted. Further, because the device is not implanted in the body, the anchoring system does not need to be designed to be atraumatic. A cartridge embodiment that fits along a conduit is contemplated without an anchoring system. Because the device is not intended to be inserted in a blood vessel, the introduction of the device to the blood stream is much simpler.

The device includes a capsule or housing that holds cell-bearing material. The cells on the cell bearing material are prevented from provoking an immunological response by isolating the cells using a porous material that creates a barrier between the blood and the cells. The barrier allows the cells to have sufficient physiological exchange (e.g., drawing sustaining nutrients and oxygen) with the blood and delivery of the metabolic products produced by the cells. The metabolic products may be in response to small messenger molecules that might be circulated in the blood stream as a result of the patient's pathologic condition. Also, the metabolic production can be stimulated by introducing a messenger molecule into the bloodstream of the patient. Additionally or alternatively, cells in devices according to the invention are also spared from opportunities of clotting. When a bloodless circuit is utilized, the primary concern of clotting is alleviated by removing contact of the device with blood. Additional concerns related to blood viscosity, nutrient concentration, and immunological response are also alleviated by allowing an altered bodily fluid, such as ultrafiltrate derived from blood or peritoneal fluid, or with peritoneal fluid itself, to contact the cells. In this case, the cells may draw nutrients and oxygen and deliver their metabolic products to these fluids. In one embodiment, with respect to, but not limited to, the chronic application of the device, an autologous cell source can be utilized. When an autologous cell source is utilized, filters placed pre- and post-device in an extracorporeal circuit optionally may or may not be used.

An embodiment of the device of the present invention includes an anchor system that secures the device to an inner wall of a extracorporeal blood circuit. The anchor system may have arms that are adapted to be in a reduced profile configuration during delivery into the blood circuit and expand into a delivered profile so that the arms expand and extend to the blood conduit. The anchoring system may use hooks, barbs, or stents disposed upon the arm of the anchor system. The anchoring system is designed such that when anchored to the wall of the blood conduit, the system permits blood in the vessel to pass through and around the device.

The device includes a shape, e.g., cone, facing upstream that minimizes turbulence of blood as it flows beyond the capsule. In one embodiment, the device comprises a semipermeable housing containing viable cells which produce and secrete the pre-selected molecule. The viable cells may be disposed on a flat surface such as a disk. In another embodiment, the cells are contained within porous tubes that allow the physiologic activity of the cells to occur and from which the preselected molecule or cell products can flow into the bloodstream or body fluids. The tubes may extend beyond the capsule.

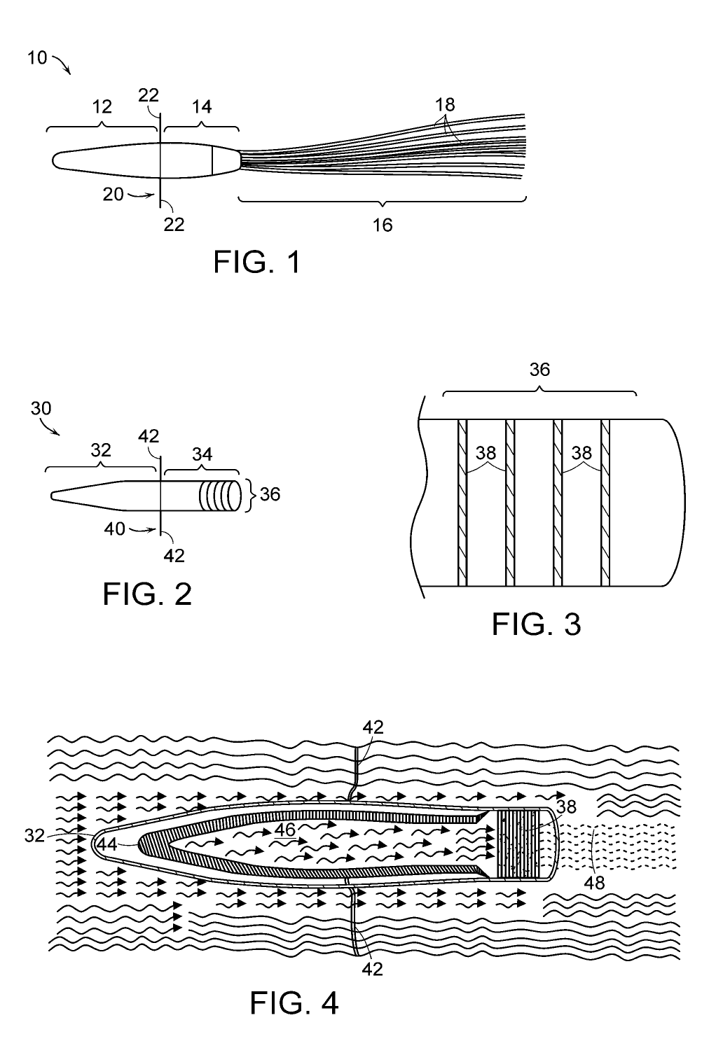

Embodiments of the present invention will now be described in greater detail with reference to the attached drawings, which are provided for purposes of illustration and are not meant to be limiting of the scope of the invention. Referring to the drawings, FIG. 1 illustrates schematically a device 10 useful in the practice of an embodiment of the present invention. In FIG. 1, the device 10 includes a nose cone 12 and a body 14 that is used to secure a cell bearing unit 16 comprised of hollow fibers 18 made of a semi-permeable material which encloses viable cells for delivering the preselected molecules. In this embodiment, the nose cone and the body of the device support the cell bearing unit 16 comprising the hollow fibers 18.

The viable cells may be attached to an inner surface of a fiber. Whether the cells are attached to the inner surface will depend upon the cell type included in the device. For example, some cell types grow in an anchorage dependent manner upon a solid surface while other cell types have no anchorage dependency and grow in suspension. The choice of cell type, however, is dependent upon the desired application.

The device of FIG. 1 is attached to an extracorporeal blood circuit by a suitable anchor system 20 that includes anchors 22. Two anchors 22 are illustrated, however, more or fewer than two could be used in a suitable design. The anchors are configured to hold the device securely in the extracorporeal conduit (shown in FIG. 8). The anchors may be retractable in a reduced profile for delivery and expanded into a deployed profile when the device is at the appropriate location on the conduit. Various springy or resilient biocompatible material may be used including nitinol or other spring material.

FIGS. 2-4 illustrate another embodiment of the invention where the device 30 includes a nose cone 32 and a body 34 into which the cell bearing unit 36 is attached. In this embodiment, the cell bearing unit includes several semi-permeable membrane disks 38 which contain the viable cells. The size and number of disks used may vary depending on the type and amount of the preselected molecule being delivered. It is contemplated that semi-permeable membranes may be defined by either the same or different polymeric compositions. An anchoring system 40 with anchors 42 is structured similarly and operates in a similar manner as the anchoring system described in connection with FIG. 1.

A detail view of the disk 38 is illustrated in FIG. 3. The disk 38 is approx 1 cm in diameter and has, in one embodiment, cells associated with the membranes such that the cells are exposed to the blood or fluid constituents for nutrients and the cells are able to deliver the desired product(s) for the blood. In a preferred embodiment, the membranes have a trabecular structure to enhance the exchange of physiologic material. In an embodiment, the disk is similar to a coin that is trabeculated and porous. The configuration allows flow-through of various media components and anchorage dependent growth especially of epithelial cells at higher tissue density.

FIG. 4 illustrates a device in a blood conduit. Blood flow is illustrated by the wavy lines. A filter 44 is provided in the device to exclude large blood constituents, e.g., macrophages and immunoglobulins, from causing an undesired immune response with the cells on the disks 38. The filtered material 46, also called ultrafiltrate, is allowed to flow across the disks 38. In other embodiments, they may flow through the membranes. The ultrafiltrate provides sustaining nutrients to the cells on the disk and carries away the products of cell metabolism. The resultant stream of ultrafiltrate blood and the products of metabolism is schematically illustrated as wavy dotted lines 48. Optionally, a filter can be used at the downstream outlet of the device.

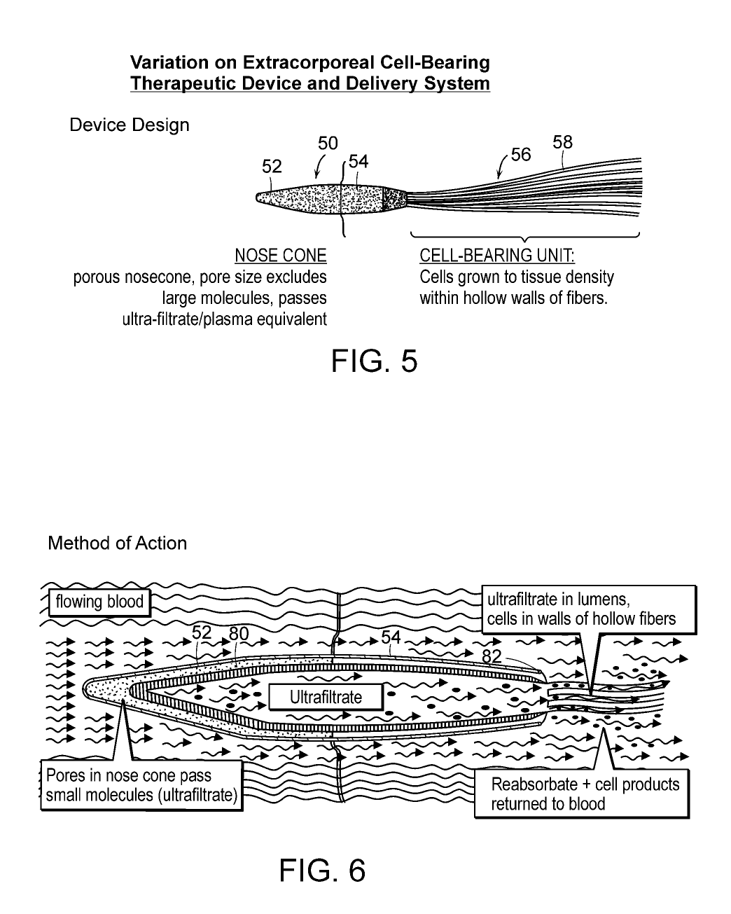

In another embodiment of the invention, illustrated in FIGS. 5-7, the hollow tubes may be constructed to provide a multi-layered structure. The device 50 includes a porous nose cone 52 that filters out large blood constituents and a body 54 that holds one end of a series of hollow tubes 56. A single hollow tube is identified by reference numeral 58.

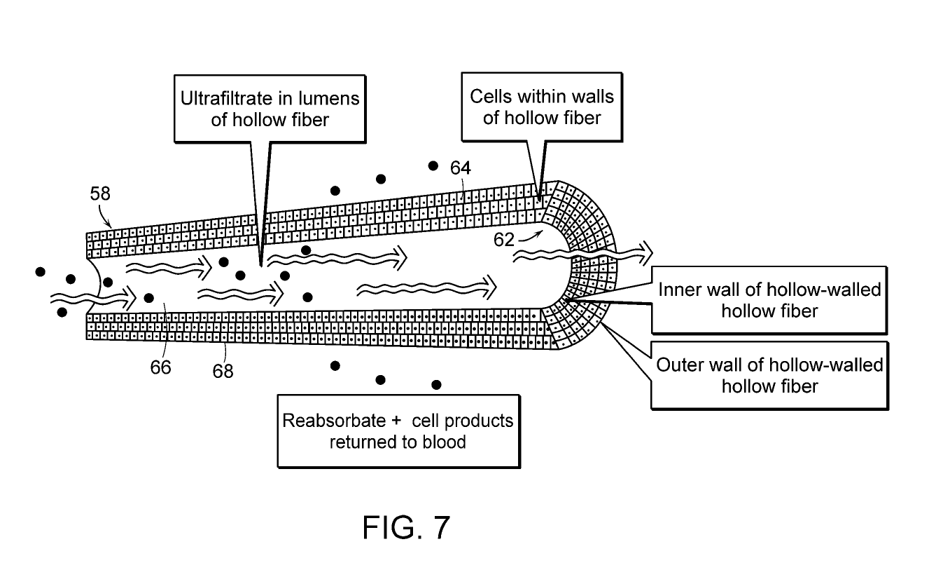

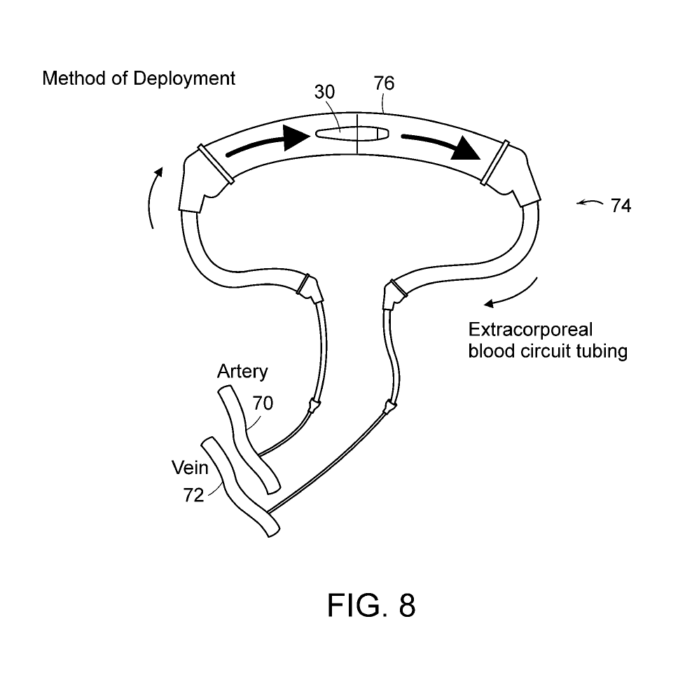

The body includes a manifold (illustrated in FIG. 6) that distributes the filtered blood into the center of the hollow tubes, one cross section of which is illustrated in FIG. 7. The distal end of the hollow tubes may be blocked (not illustrated) to provide desirable flow dynamics or to prevent the flow of unfiltered blood from being introduced to the tubes. As such, the flow path of the filtered blood can be through the tube or through the wall, depending on the desired construct. In either case, sufficient sustaining nutrients should be available to the cells within the wall of the tube.

As illustrated in FIG. 7, the hollow tube 58 includes a central lumen 62 through which the filtered blood passes. Cells 64 are disposed along a tubular medium (illustrated in axial cross section) between an inner wall 66 and an outer wall 68 and cells are disposed such that filtered blood or fluid travels across the cells. The cells are thus provided with life sustaining nutrients enabling the production of desirable cell products. In the embodiment illustrated, the outer wall of the hollow tube 58 allows the cellular products to be delivered through the tube but does not allow large particles into the tube to contact the cells. Both the inner and outer wall may have such filtering function.

Alternatively, the end of the tube is not blocked and the filtrate is allowed to bath the cells and has a sufficient flow rate such that the cells are provided with the proper amount of nutrients and the unfiltered blood is not allowed to travel upstream to the cells.

### The Anchoring System

As mentioned above, anchoring systems that may be used in the instant invention include devices that provide blood clot filtering or blood vessel stents. Useful anchoring elements are characterized by their ability to be anchored within the lumen of a conduit without occluding or preventing blood flow. One of the advantages of the present invention is that the extracorporeal device anchoring system does not have to be designed to be secured in a blood vessel without damaging the blood vessel. This minimizes the concern for tissue damage.

Blood clot filters are used routinely by medical practitioners to prevent the migration of potentially life threatening blood clots within the vasculature. Blood clot filters typically are designed to be implanted and anchored within the lumen of a blood vessel. When implanted, the anti-migration filters permit blood in the vessel to pass while simultaneously trapping blood clots. The devices and techniques useful toward attaching a filter to a blood vessel can be applied to extracorporeal devices. Of course, because the device is not intended to be introduced into the vasculature, the design of the anchors need not have the high level of precision required for their use.

Filters described in U.S. Pat. Nos. 4,817,600 and 5,059,205, referred to in the art as Greenfield® filters and available from Medi-Tech®, Boston Scientific Corporation, Natick, Mass., are particularly well suited to the practice of the invention. The cone-shaped Greenfield® vena cava filters are designed to provide maximal entrapment area for trapping blood clots while maintaining patency of the blood vessel after trapping emboli. The spacing between the six legs of the Greenfield® vena cava filters ensures the trapping of emboli greater than 3 mm (Greenfield et al. (1989) “Venous Interruption” Chapter 68, pp. 929-939 in “Haimovici's Vascular Surgery Principles and Techniques, Third Edition,” Appleton and Lange, Norwalk, Conn./San Mateos, Calif.). Accordingly, the filters may be able to capture capsules greater than 3 mm in diameter. In another embodiment of the invention, the device is incorporated into a tube that is inserted into an extracorporeal conduit. In this embodiment, the anchors are unnecessary because the device is secured within the tube.

### Device Design

The extracorporeal drug delivery device of the various embodiments of the present invention may be capable of delivering a preselected drug or cell products over an extended period of time. Because the device is not designed to be implanted, the size of the capsule can be scaled for higher delivery rates by increasing the size of the capsule and the drug delivery media.

Because the device permits delivery of the preselected molecule over defined periods of time, another important consideration in the design of the device is the configuration that will maintain the viability of the cells enclosed in the device. It is understood that a variety of factors, for example: the supply of oxygen and nutrients to the cells in the capsule; the removal of waste products from the cells in the capsule; the minimization of host immune responses directed against the cells in the capsule; the proliferative activity of the cells; and whether cells located at the center of the capsules are susceptible to pressure necrosis, all of which may influence the design and preparation of a cell containing tube(s).

In addition to adequate aeration, it is important that the encapsulated cells obtain sufficient amounts of essential nutrients from the blood supply to remain viable. The transport oxygen is almost entirely by diffusion and this is a significant aspect in maintaining cell viability. Once the geometry of a hollow fiber has been optimized for oxygen transport, then the hollow fiber inherently will be able to permit the diffusion of adequate amounts of nutrients into the lumen of the capsule from the blood stream. Similarly, such a geometry is contemplated also to permit diffusion of cell metabolites, including, waste products and the preselected molecule, out of the hollow fiber and into the blood stream.

The hollow fibers preferably are produced from a semi-permeable membrane having pores dimensioned to permit the diffusion of oxygen and nutrients into the lumen of the hollow fiber while permitting the efflux of cellular waste products and the pre-selected molecule out of the hollow fiber. In addition, the pores preferably are dimensioned to exclude the passage of cells therethrough. Accordingly, the pores are designed to prevent migration of the viable cells from the lumen of the hollow fiber into the blood steam, thereby maintaining the implanted cells at a single location in the host to facilitate their subsequent removal if or when necessary. The pores also are designed to prevent the influx of the hosts immune cells, for example, macrophages and lymphocytes, which if allowed to enter the lumen of the hollow fibers may be detrimental to the viability of the cells enclosed therein. The membrane, therefore, provides an immuno-protected environment that protects cells enclosed therein from an immune response. This may be an important consideration if the implanted cells are non-autologous in nature. If autologous cells are used per size restrictions longer than molecular dimension then the design would be modified accordingly.

The hollow fibers comprising, or for incorporation within, the capsule may be produced from biocompatible polymers which include, but are not limited to, polyvinylchloride, polyvinylidene fluoride, polyurethane isocyanate, polyalginate, cellulose acetate, cellulose diacetate, cellulose triacetate, cellulose nitrate, polysulfone, polystyrene, polyurethane, polyvinyl alcohol, polyacrylonitrile, polyamide, polymethylmethacrylate, polyethylene oxide, polytetrafluoroethylene, or copolymers thereof. A summary of currently available hollow fibers, including methods of manufacture and the names of commercial suppliers, is set forth in Radovich (1995) “Dialysis Membranes: Structure and Predictions” Contrib Nephrol., Basel, Karger, 113:11-24, the entire disclosure of which is incorporated herein by reference. In addition, polytetrafluorethylene polymer hollow fibers are available commercially from Impra, Inc., Tempe, Ariz. or W.L. Gore and Associates, Flagstaff, Ariz. U.S. Pat. No. 6,716,208, the contents of which is incorporated by reference, provide more details of the components of the device.

### Disks

The disks provide a suitable material onto which the cells can be disposed. In a preferred form, the disks have a trabecular structure that allows the cells to grow into the medium. Alternatively, the cells may be disposed on the surface of the membrane. The disk material may consist of a variety of different types of compounds, including ceramics, carbon, and metallic substances. In one embodiment, the disks are made from carbon material coated with niobium (Nb). In certain embodiments, the disk material may be a porous, metal-coated reticulated open cell foam of carbon-containing material, the metal coating selected from, but not limited to, tantalum, titanium, platinum (including other metals of the platinum group), Nb, hafnium, tungsten, and combinations thereof. The coatings (which may be non-metal as well) also may include, but are not limited to, silica, nickel, alumina, gold, collagen, non-siliceous materials, and nanofibrous scaffolds, complexes, composites, or constructs formed by any variety of methods including, but not limited to, nano-magnetic deposition, nanocasting, fiberbonding, electrospinning, or any variation on these techniques. The coatings can increase integrity and strength of the disks. Alternatively, other shapes of substrates on and/or in which cells are disposed may be used, such as rectangular or square substrates. Further, the substrates can be made from various polymers, which may have a trabeculated structure. The polymers can promote or increase cell expansion, attachment, and/or viability.

### Use of the Device

Cell therapy presents itself as a new approach to the treatment of acute and chronic diseases. This therapeutic approach has its origins in the growing appreciation that most disease processes are not due to the lack of a single protein but develop due to alterations in complex interactions of a variety of cell products. Cell therapy depends on cell and tissue culture methodologies to expand specific cells to replace important differentiated processes deranged or lost in various disease states. Recent approaches have made progress by placing cells into hollow fiber bioreactors or encapsulating membranes as a means to deliver cell activities to a patient, requiring complex extracorporeal pump systems and large bioreactor devices. One clinical disorder that may be treatable with cell therapy is the systemic inflammatory response syndrome, or SIRS. It is a catastrophic sequela of a variety of clinical insults, including infection, pancreatitis, and cardiopulmonary bypass, and claims over forty thousand lives in the U.S. each year. The most common cause of SIRS is bacteria-induced septic shock. The exceptionally high mortality associated with the syndrome is due in part to the development of the highly lethal multiple system organ failure syndrome (MOF) in a subset of patients with SIRS.[references 1,2]

One area that presents opportunities is the development of miniaturized cell therapy devices containing renal tubule cells, which play an important immunologic regulatory role in septic shock. The successful development of a prototype is an important step toward allowing cell therapy to be delivered in a simple extracorporeal cassette without extracorporeal pump systems, thereby expanding this therapy to a broader indication, especially early severe sepsis syndrome prior to established acute renal failure (ARF).

An extracorporeal device utilizing a standard hemofiltration cartridge seeded with approximately 10⁸ renal tubule cells grown from adult stem/progenitor cells as confluent monolayers along the inner surface of the fibers has been successfully fabricated with human cells.[reference 4] This initial cell therapy device is large (12×4-in cylinder) and requires an additional extracorporeal pump circuit to deliver blood and plasma ultrafiltrate to the cell-containing device. These elements were designed as an add-on to current dialysis treatment in patients with ARF.

The successful creation of the miniature device proposed has the potential to lead to a variety of other cell therapy devices including “wearable artificial organs”. Current cell therapy approaches target the use of stem cells for neurodegenerative disorders (Parkinson's, Alzheimer's), spinal cord injury, heart disease (congestive heart failure, myocardial infarct), pancreas disease (diabetes), liver disease (cirrhosis, hepatitis), kidney disease (end-stage renal disease [ESRD], ARF), blood (sickle cell anemia), muscle disorders (muscular dystrophy), skin (burns), and bones (arthritis, osteoporosis). From a market standpoint, the value of cell-based markets is estimated to be $26.5 billion in 2005, $56.0 billion in 2010, and $96.0 billion in 2015. More than 300 companies are involved in cell therapy. The near-term market for the use of renal cell devices is also very large and includes (U.S. only): ARF (100,000 patients, $2 billion market), ESRD (400,000 patients, $20 billion cost), and cardiorenal syndrome (350,000 patients, $25 billion cost).

Any of the embodiments of devices for use in extracorporeal circuits according to the invention (whether or not utilizing pumps and/or fluid separation devices) can be used to treat the disease states described herein (such as the cell therapy targets described above). Moreover, any type of cell can be used with any of the embodiments of devices for use in extracorporeal circuits according to the invention, including, for example, a eukaryotic cell, such as a mammalian cell, such as a renal cell (e.g., a renal tubule cell). The cells can treat bodily fluids in order to prevent or alleviate the disease conditions described herein.

### Extracorporeal Treatment Devices

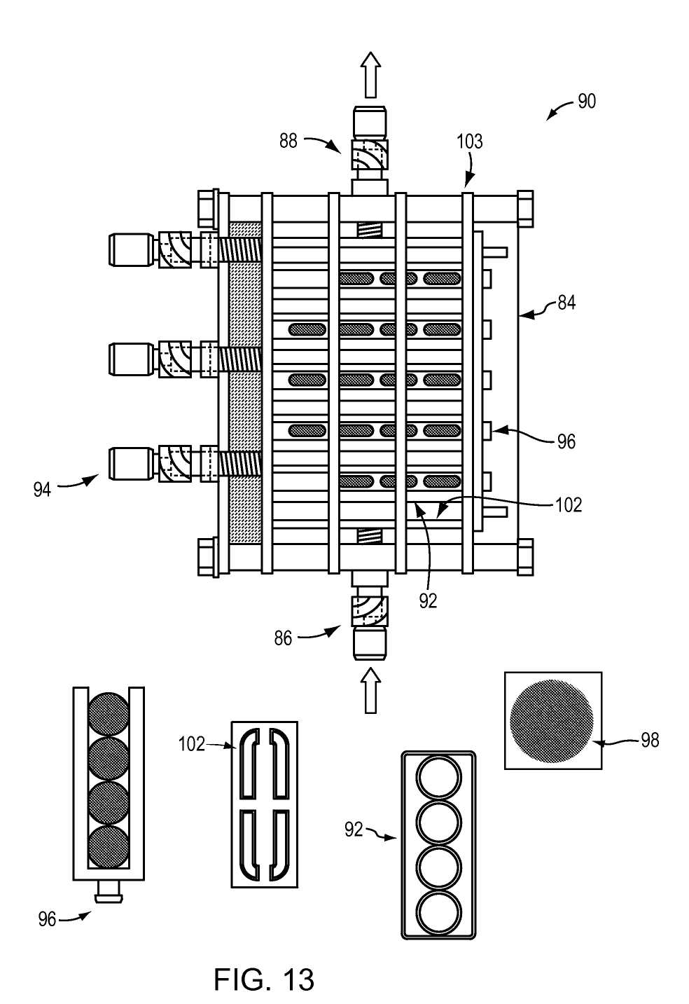

FIG. 13 depicts another embodiment of an extracorporeal treatment device where the device 90 includes a housing 84 defining an interior space. Gaskets 103 interface with the housing 84 to seal the housing. There are four gaskets shown in this embodiment, but devices may have fewer or more than shown. The housing 84 includes an inlet 86 for receiving a fluid and an outlet 88 for releasing a processed fluid. Within the interior space of the housing 84, viable cells are disposed on a substrate, in this case on disks 98. The cells can be any that are described herein. In this embodiment, the disks 98 are constructed from carbon material coated with Nb, have a trabeculated shape and are porous. Optionally, the disks 98 also are coated with a biomatrix material, such as, for example, collagen IV. The disks either can be initially separate from the housing 84 and inserted therein or can be shipped to a customer with the disks in place. Scaffolds 96 retain the disks 98 within the interior space of the housing 84, and are shaped like a tuning fork in this embodiment. There are five scaffolds shown in this embodiment, but devices may have fewer or more of the them than shown. Additionally, each scaffold may hold fewer or more disks. A flow separator 102 within the interior of the housing 84 is disposed between the inlet 86 and the disks 98. When fluid enters the housing 84 through the inlet 86, the flow separator 102 provides desirable flow dynamics to allow the fluid to pass through the disks 98 and adjacent the viable cells. Additionally, baffles 92 are disposed between the inlet 86 and the disks 98 to further direct the fluids through the disks 98. Each of the baffles 92 is adjacent or proximate to a scaffold 96. The housing 84 also includes ports 94. The ports 94 allow for pressure release, fluid expansion, extracorporeal flow, and analysis probes. Additionally, the device may have fewer, more, or no ports.

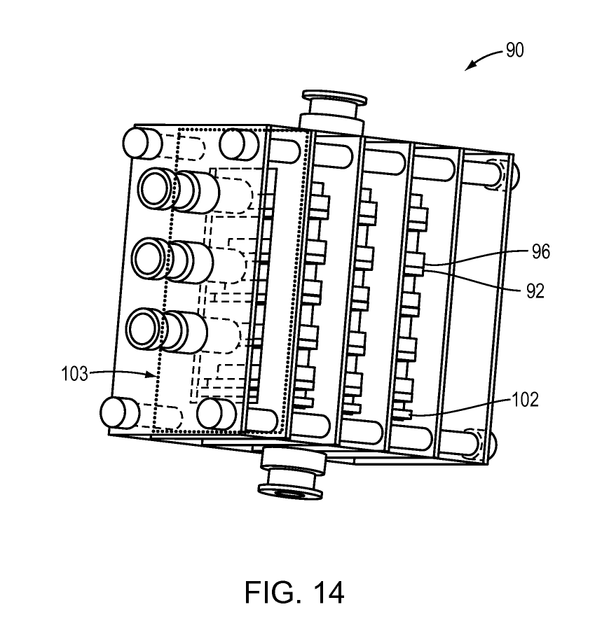

FIG. 14 illustrates schematically another view of FIG. 13. The gaskets 103 have a substantially rectangular, annular shape with a rectangular aperture therethrough, and are placed about the interior space of the housing. Additionally, the gaskets can have other shapes, including the apertures formed therethrough.

The embodiment illustrated in FIGS. 13 and 14 distributes a fluid through the disks and adjacent the viable cells. As the fluid flows into the housing 84 through the inlet 86, the flow separator 102 is designed to prevent the fluid from solely flowing through the center of the housing. Rather, as shown in the embodiment in FIG. 13, the flow separator 102 has four apertures that distribute the fluid to the front, back, and sides of the housing 84. There are four apertures shown in this embodiment, but flow separators may have fewer or more apertures of various shapes. As the fluid passes through the flow separator 102, the fluid next encounters a baffle 92. As shown in the embodiment in FIG. 13, each baffle 92 has apertures that correspond to the number and shape and size of the disks 98; here, each baffle 92 has four circular apertures. The apertures of the baffles 92 are designed to allow the fluid to pass through the disks 98 and adjacent the viable cells. Additionally, baffles may have fewer or more apertures of various shapes. After the fluid passes through each of the baffles 92 and disks 98 throughout the housing 84, the processed fluid exits the housing 84 through the outlet 88. Fluids that may pass through this device include ultrafiltrate (derived from blood and/or peritoneal fluid) and peritoneal fluids (including peritoneal dialysate).

The materials of the embodiment illustrated in FIGS. 13 and 14 are biocompatible. For example, the materials should not leach anything upon contact with a liquid, and toxins should not make contact with biological fluid. The materials can be sterilized with the use of, but not limited to, ethylene oxide, an autoclave, gamma radiation, cold sterilant, or any combination thereof. Useful materials also should not crack or substantially change when transitioning from various temperatures, such as a change to room temperature. The materials may include both virgin materials and materials with biocompatible coatings. For example, the housing may be produced from a biocompatible material such as polycarbonate, polysulfone, or acrylic. Also, the baffles and flow separator may be produced from a material which includes, but is not limited to, polycarbonate, polysulfone, or acrylic. The scaffolds may be produced from a material such as polypropylene. Further, the gaskets may be produced from a material such as ethylene propylene diene monomer, tetrafluoroethylene perfluoromethyl vinyl ether, polysteel, or silicone rubber. The substrates on which the cells are located (in this case, the disks) can be made from a variety of materials, including ceramics, carbon, and metallic substances. For example, the substrate may be made from a porous, metal-coated reticulated open cell foam of carbon-containing material, the metal coating selected from, but not limited to, tantalum, titanium, platinum (including other metals of the platinum group), Nb, hafnium, tungsten, and combinations thereof. The coatings (which may be non-metal as well) also may include, but are not limited to, silica, nickel, alumina, gold, collagen, non-siliceous materials, and nanofibrous scaffolds, complexes, composites, or constructs formed by any variety of methods including, but not limited to, nano-magnetic deposition, nanocasting, fiberbonding, electrospinning, or any variation on these techniques. Alternatively, other shapes of substrates on and/or in which cells are disposed may be used, such as rectangular or square substrates. Further, the substrates can be made from various polymers, which may have a trabeculated structure. The polymers can promote or increase cell expansion, attachment, and/or viability. Additionally, the components of the embodiment in FIGS. 13 and 14 may be produced from a biocompatible material which includes, but is not limited to, polycarbonate, polystyrene, high density polyethylene, low density polyethylene (such as any polyethylene composite), polysulfone, polypropylene, stainless steel, aluminum, titanium alloy, acrylic, syndiotactic polystyrene (such as any polystyrene composite), high impact polystyrene, ultra high molecular weight polyethylene, polyamides, polybutylene terephthalate, polyester alloy, polyetherimide, polyetheretherketone, polyphenlene ether, polyether sulfone, polyphenylsulfone, thermoplastic polyurethane, polyvinyl chloride, polytetrafluoroethylene, perfluoro alkoxy, silicone rubber, ethylene propylene diene monomer, polyvinyl fluoride, ethylene-tetrafluoroethylene, fluorinated ethylene propylene, ethylene-chlorotrifluoroethylene, polychloroprene, fluorosilicone, tetrafluoroethylene perfluoromethyl vinyl ether, tetrafluoroethylene propylene copolymer, thermoplastic elastomer (such as olefinic), styrenic thermoplastic elastomer, thermoplastic polyester urethane elastomer, copolyester ether elastomer, terlux, tritan, delrin, polyurethane adhesive, silicone adhesive, helixone, or copolymers thereof.

The substrate in this embodiment can be shipped to the site of use with cells grown on the substrate and in a cryopreserved state. When the medical professional wishes to use the device, the substrate, and the cells thereon, can be thawed and loaded into the device. This configuration facilitates ease of manufacturing, shipping, and storage, as the pre-grown cells on the substrate can be shipped and stored in a frozen state.

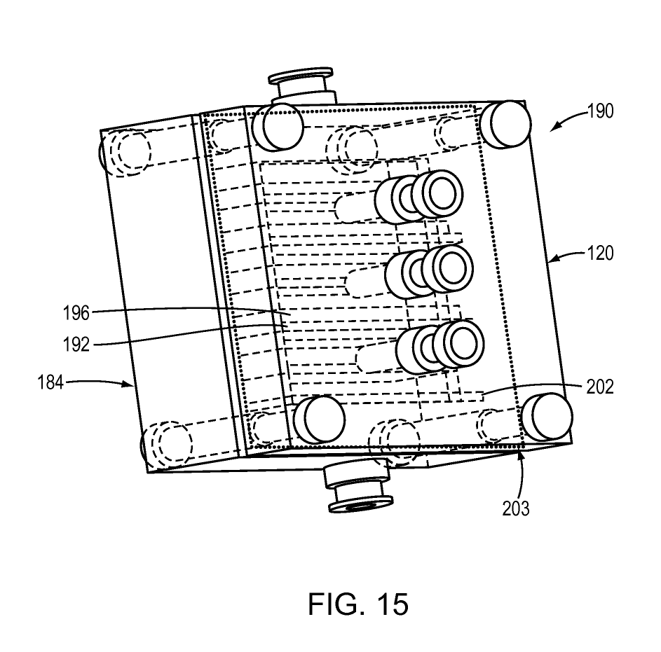

FIG. 15 illustrates schematically another embodiment where the entire device 190 can be freezable or cryopreserved. In this embodiment, the device 190 includes a housing 184 defining an interior space, a lid 120 to cover the housing 184, and a gasket 203 to seal together the lid 120 and the housing 184. The gasket 203 has a substantially rectangular, annular shape with a rectangular aperture therethrough, and is placed about the interior space of the housing. Additionally, the gasket can have other shapes, including the apertures formed therethrough. Alternatively, two gaskets may be used to maintain a seal.

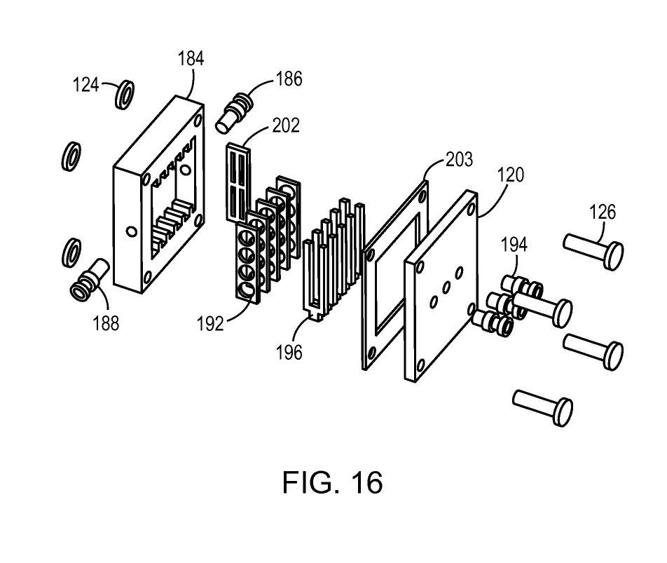

FIG. 16 illustrates an exploded view of the embodiment shown in FIG. 15. The housing 184, lid 120, and gasket 203 are held together by nuts 124 and bolts 126, which are placed in grooves of the housing 184, lid 120, and gasket 203. In certain situations, each bolt can be approximately 1 inch without interior threading. Alternatively, the bolts can be longer or shorter or with interior threading. The housing 184 includes an inlet 186 for receiving a fluid and an outlet 188 for releasing a processed fluid. Within the interior space of the housing 184, viable cells are disposed on a substrate, in this case on disks (not shown). In this embodiment, the disks are constructed from carbon material coated with Nb, have a trabeculated shape and are porous. Each disk can have a diameter of 8 mm, although the size of the disk can be greater or lesser in size. Optionally, the disks also are coated with a biomatrix material, such as, for example, collagen IV. Alternatively, the disks may be constructed from ceramics or metallic substances, or the disk may be a porous, metal-coated reticulated open cell foam of carbon-containing material, the metal coating selected from tantalum, titanium, platinum (including other materials of the platinum group), Nb, hafnium, tungsten, and combinations thereof. Alternatively, other shapes of substrates on and/or in which cells are disposed may be used, such as rectangular or square substrates. Further, the substrates can be made from various polymers, which may have a trabeculated structure. The polymers can promote or increase cell expansion, attachment, and/or viability. This embodiment is typically shipped to a customer in a frozen state with the disks in place. Again, this provides a benefit in manufacturing, shipping, and storage such that the completed device, with pre-grown cells in place, can be shipped in a frozen state to the site of use. A medical professional then thaws the entire device prior to use.

Scaffolds 196 retain the disks within the interior space of the housing 184, and are shaped like a tuning fork in this embodiment. There are five scaffolds shown in this embodiment, but devices may have fewer or more of the them than shown. Additionally, each scaffold may hold any number of disks. A flow separator 202 within the interior of the housing 184 is disposed between the inlet 186 and the disks. When fluid enters the housing 184 through the inlet 186, the flow separator 202 provides desirable flow dynamics to allow the fluid to pass through the disks and adjacent the viable cells. Additionally, baffles 192 are disposed between the inlet 186 and the disks to further direct the fluids through the disks. Each of the baffles 192 is adjacent or proximate to a scaffold 196. The housing 184 also includes ports 194. The ports 194 allow for pressure release, fluid expansion, extracorporeal flow, and analysis probes. Additionally, the device may have fewer, more, or no ports. In this figure, the inlet 186, outlet 188, and ports 194 are luer fittings. These components can be selectively attached or detached from tubing or grooves. Alternatively, the inlet 186, outlet 188, and ports 194 need not be luer fittings. In certain situations, an adhesive (not shown) can be used with luer fittings, such as the inlet 186, outlet 188, and ports 194 in this figure, to further maintain a seal.

The embodiment illustrated in FIGS. 15 and 16 distributes uniformly a fluid through the disks and adjacent the viable cells. As the fluid flows into the housing 184 through the inlet 186, the flow separator 202 is designed to prevent the fluid from solely flowing through the center of the housing. Rather, as shown in the embodiment in FIG. 16, the flow separator 202 has four apertures that distribute the fluid to the front, back, and sides of the housing 184. There are four apertures shown in this embodiment, but flow separators may have fewer or more apertures of various shapes. As the fluid passes through the flow separator 202, the fluid next encounters a baffle 192. As shown in the embodiment in FIG. 16, each baffle 192 has four circular apertures that correspond to the number and shape and size of the disks. The apertures of the baffles 192 are designed to allow the fluid to pass through the disks and adjacent the viable cells. Additionally, baffles may have fewer or more apertures of various shapes. After the fluid passes through each of the baffles 192 and disks throughout the housing 184, the processed fluid exits the housing 184 through the outlet 188. Fluids that may pass through this device include ultrafiltrate (derived from blood and/or peritoneal fluid) and peritoneal fluids (including peritoneal dialysate).

The materials of the embodiment illustrated in FIGS. 15 and 16 are biocompatible. For example, the materials should not leach anything upon contact with a liquid, and toxins should not make contact with biological fluid. The materials can be sterilized with the use of, but not limited to, ethylene oxide, an autoclave, gamma radiation, cold sterilant, or any combination thereof. Useful materials also should not crack or substantially change when transitioning from various temperatures, such as a change to room temperature. The materials may include both virgin materials and materials with biocompatible coatings. For example, the housing may be produced from a biocompatible material such as polycarbonate, polysulfone, or acrylic. Also, the baffles and flow separator may be produced from a material which includes, but is not limited to, polycarbonate, polysulfone, or acrylic. Further, the gasket or gaskets may be produced from a material which includes, but is not limited to, ethylene propylene diene monomer, tetrafluoroethylene perfluoromethyl vinyl ether, polysteel, or silicone rubber. The substrates on which the cells are located (in this case, the disks) can be made from a variety of materials, including ceramics, carbon, and metallic substances. For example, the substrate may be made from a porous, metal-coated reticulated open cell foam of carbon-containing material, the metal coating selected from, but not limited to, tantalum, titanium, platinum (including other metals of the platinum group), Nb, hafnium, tungsten, and combinations thereof. The coatings (which may be non-metal as well) also may include, but are not limited to, silica, nickel, alumina, gold, collagen, non-siliceous materials, and nanofibrous scaffolds, complexes, composites, or constructs formed by any variety of methods including, but not limited to, nano-magnetic deposition, nanocasting, fiberbonding, electrospinning, or any variation on these techniques. Additionally, the bolts may also be constructed from a material which includes, but is not limited to, stainless steel. The scaffolds may be produced from a material such as polypropylene. The inlet, outlet, and ports may be produced from a material which includes, but is not limited to, polycarbonate or polypropylene. The adhesive may be produced from a material which includes, but is not limited to, polyurethane adhesive or silicone adhesive. Furthermore, the components of the embodiment in FIGS. 15 and 16 may be produced from a biocompatible material which includes, but is not limited to, polycarbonate, polystyrene, high density polyethylene, low density polyethylene (such as any polyethylene composite), polysulfone, polypropylene, stainless steel, aluminum, titanium alloy, acrylic, syndiotactic polystyrene (such as any polystyrene composite), high impact polystyrene, ultra high molecular weight polyethylene, polyamides, polybutylene terephthalate, polyester alloy, polyetherimide, polyetheretherketone, polyphenlene ether, polyether sulfone, polyphenylsulfone, thermoplastic polyurethane, polyvinyl chloride, polytetrafluoroethylene, perfluoro alkoxy, silicone rubber, ethylene propylene diene monomer, polyvinyl fluoride, ethylene-tetrafluoroethylene, fluorinated ethylene propylene, ethylene-chlorotrifluoroethylene, polychloroprene, fluorosilicone, tetrafluoroethylene perfluoromethyl vinyl ether, tetrafluoroethylene propylene copolymer, thermoplastic elastomer (such as olefinic), styrenic thermoplastic elastomer, thermoplastic polyester urethane elastomer, copolyester ether elastomer, terlux, tritan, delrin, polyurethane adhesive, silicone adhesive, helixone, or copolymers thereof.

The embodiment of the present invention illustrated in FIGS. 15 and 16 accomplishes freezing or cryopreservation of the entire device 190 in order to maintain cell viability. For example, the device 190 can be viable in a liquid nitrogen vapor phase at a temperature range of negative 90 to negative 150 degrees Celsius; alternatively, the device 190 can be stored in the liquid phase at negative 190 degrees Celsius. The device 190 is also biocompatible, maintains seal without leaking or cracking, and withstands sterilization and pressure. The device 190 may be stored in a sterile cryo-compatible bag for freezing and thawing. Additionally or alternatively, the device 190 may incorporate (temporarily or permanently) a one way outward valve at the inlet. The valve allows for outlet venting during the freeze/thaw process while keeping the outside, non-sterile air from entering the device.

The embodiments of the present invention illustrated in FIGS. 13-16 may also include a pre- and/or a post-filtration unit integrated with the housing, thus creating a porous barrier between the viable cells and the body fluid flowing into the treatment device. The filtration unit prevents the cells within the housing from provoking an immunological response and produces an ultrafiltrate that provides nutrients and oxygen to the cells. Additionally, in the embodiments shown in FIGS. 13-16, the flow separator can either be separate from or integrated with the housing. Further, the substrate on which the cells are disposed can be any shape, such as rectangular or square.

Devices of the present invention as illustrated in FIGS. 13-16 may be sealed with a snap-fit or screw-fit design rather than using gaskets, nuts, and/or bolts. In addition, the snap-fit or screw-fit design can incorporate a holding system for the substrates (e.g., disks) into the housing, such as a combined scaffold/baffle assembly. Further, the design can have a flow separator integrated with the housing.

The embodiments of the present invention illustrated in FIGS. 13-16 can employ the use of dynamic culture and seeding methods, such as, for example, for renal cell therapy. The disks of the embodiments of FIGS. 13-16 can be cultured under rotational flow. Further, the disks can be fated to the devices 90, 190 of FIGS. 13-16 either post seeding or after about two weeks in a dynamic culture system (such as a dynamic Starwheel culture system). The devices 90, 190 can be incorporated into a bloodless circuit for chronic or acute use.

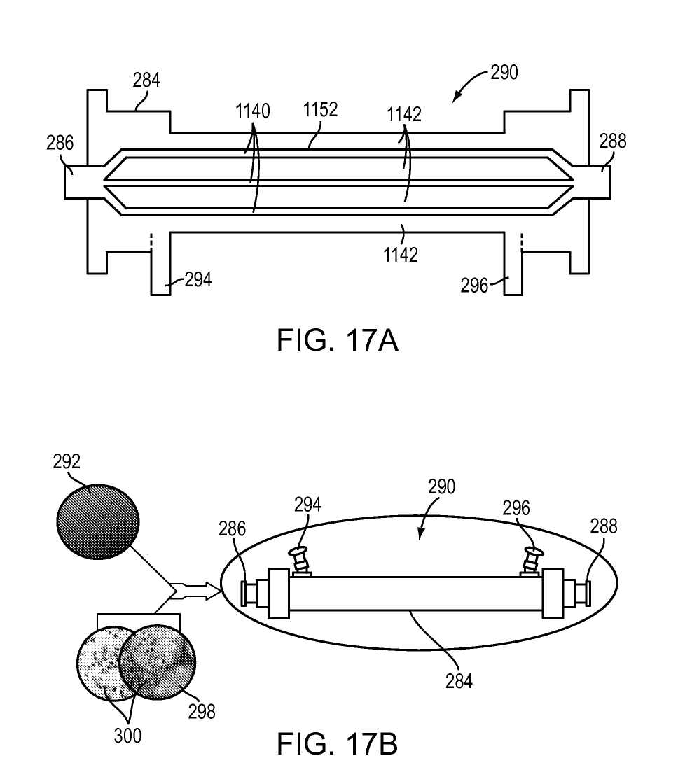

FIGS. 17A and B depict another embodiment 290 of the present invention where viable cells are seeded in hollow fibers 1152 contained within a housing 284. The housing 284 includes an inlet 286 for receiving a fluid, an outlet 288 for releasing a processed fluid, and two ports 294, 296. Hollow fibers 1152 connect the inlet 286 and the outlet 288. The hollow fibers 1152 define a luminal space referred to as an intracapillary space (ICS) 1140. The space surrounding the hollow fibers 1152 is the extracapillary space (ECS) 1142 and is within the housing 284. In certain situations, an adhesive (not shown) can be used to occupy the spaces between the hollow fibers 1152 at the inlet 286 and at the outlet 288 of the device 290. The adhesive holds the hollow fibers 1152 in place and defines the ICS and the ECS of the device 290. The device 290 can sustain cells 300 for incorporation into ex vivo circuits or implantation.

Viable cells 300 are seeded within the ICS 1140 of at least one hollow fiber 1152. The cells 300 can be adherent cells. In certain embodiments, the cells 300 are grown on particles 298, such as beads or other microcarriers, and are seeded into the ICS 1140 while on the particles 298. Examples of microcarriers include, but are not limited to, HyQ® Spheres™ microcarriers (available from HyClone, Inc., South Logan, Utah and SoloHill Engineering, Inc., Ann Arbor, Mich.), Cultispher® microcarriers (available from HyClone, Inc. South Logan, Utah), Cytodex® microcarrier beads (available from GE Healthcare Bio-Sciences Corp., Piscataway, N.J.), and Glass microcarrier beads (available from Sigma-Aldrich, Inc., St. Louis, Mo.). The HyQ® Spheres™ microcarrier is a cross-linked, polystyrene microcarrier. It can be optionally coated with collagen or animal-derived component free materials. Further, it can have a cationic charge layering surface modification. The Cultispher® microcarriers have larger surface areas for attachment and are dissolvable for cell retention. They come in three types (G: basic gelatin cross linked, S: large pore cross linked, and GL: high temperature cross-linked). The Cytodex® microcarrier bead is a dextran, cross-linked microcarrier. It has positively charged Diethylaminoethyl (DEAE) groups throughout the matrix. The Glass microcarrier beads can be of varying size and density.

When the cells 300 are within the device 290, they are located within a biomatrix material 292, which itself is disposed within the ICS 1140. Thus, the cells are suspended in the biomatrix material 292 or physiologic buffer (e.g., a 3-D matrix suspension) in the hollow fibers 1152. Examples of a biomatrix material 292 or physiologic buffer include, but are not limited to, alginate solution, gelled alginate solution, nutragen, matrigel, collagen I or any three-dimensional forming or formed biocompatible matrix.

The hollow fibers 1152 of this or other hollow-fiber embodiments of the invention can have any of the following design characteristics. The hollow fibers can have immunoprotective properties, and cells can be cultured on the inside of the fibers. The pores through the hollow fibers can be of a size within the range of about 0.01 μm to about 0.1 μm. Pore sizes also can be measured as a molecular weight cutoff (MWCO) and can be of a type useful for ultrafiltration. MWCO and size are loosely related with 0.01 μm equating to a MWCO of about 70,000 kD and 0.1 μm equating to a MWCO of about 500,000 kD. The hollow fibers typically have a MWCO of about 40,000-50,000 kD. The hollow fibers can function as a barrier between the nutrient supply source and the cells.

The biomatrix material and/or cells can be inserted into the device at the point of manufacture or, alternatively, at the clinical site. If the cells are inserted at the point of manufacture, the device can be frozen and delivered in such a state. Then, the entire device is thawed prior to use by the medical professional. If the cells are inserted at the clinical site, the cells can be cryopreserved separately from the device. Then, the cryopreserved cells are thawed at the clinical site, when they are to be used, mixed with the biomatrix material and injected (e.g., with a syringe) into the device. Alternatively, the cryopreserved cells can be pre-mixed with the biomatrix material and both components can be thawed when used. In one embodiment, cryopreserved cells, grown on microcarriers, are thawed and, if necessary, mixed with a three-dimensional biomatrix, such as alginate. This mixture is injected into the device, and the mixture is allowed to gel. In the case of alginate, the addition of calcium chloride is beneficial for gel formation, although this step will vary depending on the biomatrix material and does not limit any three-dimensional matrix utilization. Alternatively, a three dimensional matrix may be excluded.

The materials of the embodiment illustrated in FIGS. 17A and B are biocompatible. For example, the materials should not leach anything upon contact with a liquid, and toxins should not make contact with biological fluid. The materials can be sterilized with the use of, but not limited to, ethylene oxide, an autoclave, gamma radiation, cold sterilant, or any combination thereof. Useful materials also should not crack or substantially change when transitioning from various temperatures, such as a change to room temperature. The materials may include both virgin materials and materials with biocompatible coatings. For example, the housing or cartridge may be produced from a biocompatible material such as polycarbonate. Also, the hollow fibers (e.g., semi-permeable encapsulating membranes) may be produced from a material which includes, but is not limited to, polysulfone, poloyetherimide, polyether sulfone, helixone, polyalginate, polyvinylchloride, polyvinylidene fluoride, polyurethane isocyanate, cellulose acetate, cellulose diacetate, cellulose triacetate, cellulose nitrate, polysulfone, polystyrene, polyurethane, polyvinyl alcohol, polyacrylonitrile, polyamide, polymethylmethacrylate, polytetafluoroethylene, polyethylene oxide, or copolymers thereof. The inlet, outlet, and ports may be produced from a material which includes, but is not limited to, polycarbonate or polypropylene. The adhesive may be produced from a material which includes, but is not limited to, polyurethane adhesive or silicone adhesive. Furthermore, the components of the embodiment in FIGS. 17A and B, with the exception of the viable cells, may be produced from a biocompatible material which includes, but is not limited to, polycarbonate, polystyrene, high density polyethylene, low density polyethylene (such as any polyethylene composite), polysulfone, polypropylene, stainless steel, aluminum, titanium alloy, acrylic, syndiotactic polystyrene (such as any polystyrene composite), high impact polystyrene, ultra high molecular weight polyethylene, polyamides, polybutylene terephthalate, polyester alloy, polyetherimide, polyetheretherketone, polyphenlene ether, polyether sulfone, polyphenylsulfone, thermoplastic polyurethane, polyvinyl chloride, polytetrafluoroethylene, perfluoro alkoxy, silicone rubber, ethylene propylene diene monomer, polyvinyl fluoride, ethylene-tetrafluoroethylene, fluorinated ethylene propylene, ethylene-chlorotrifluoroethylene, polychloroprene, fluorosilicone, terlux, tritan, delrin, polyurethane adhesive, silicone adhesive, helixone, or copolymers thereof.

### Extracorporeal Circuits for Treatment Devices

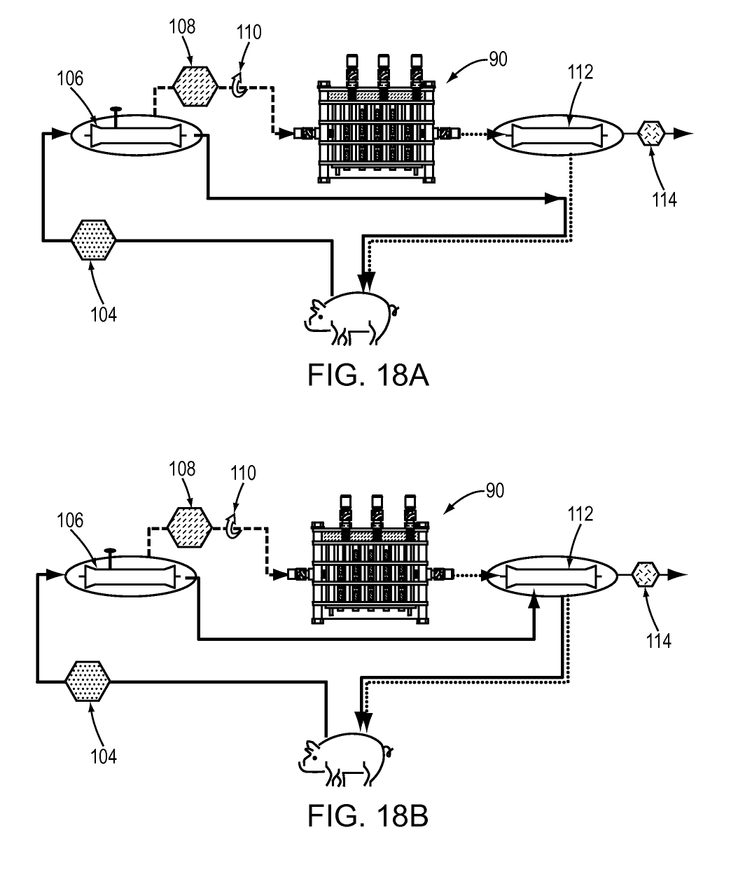

The extracorporeal devices described herein can be used with extracorporeal circuits as illustrated in FIGS. 18A and B (FIGS. 18A and B show the device of FIG. 13, but the circuits can be used with other devices according to the invention, such as, for example, the devices of FIGS. 15 and 16). The blood from a subject (e.g., an animal or a human) is pumped by a first pump 104. The blood is then circulated through a first hemofilter 106 having hollow fibers therein defining an ICS and an ECS. The blood passes through the ICS, and is split into a filtered blood fraction and an ultrafiltrate fraction. The flow rate of ultrafiltrate generated by the first hemofilter 106 is controlled by a second pump 108 that passes the ultrafiltrate from the first hemofilter, through a thermal regulator 110 for maintenance of temperature within the device 90 illustrated in FIG. 13, into the device 90 where the ultrafiltrate flows across the viable cells on the disks 98, and into a second hemofilter 112. The second hemofilter 112 outlet flow rate is controlled by a third pump 114 to allow for maximal ultrafiltrate filtration to pass through to the ECS of the second hemofilter 112, while maintaining physiologic pressures. In FIG. 18A, upon exiting the second hemofilter 112, the processed ultrafiltrate is combined with the filtered blood, which came directly from the first hemofilter 106, and is returned to the subject. In FIG. 18B, the filtered blood from the first hemofilter 106 flows directly to the second hemofilter 112 and is combined with the processed ultrafiltrate. The combined processed ultrafiltrate and filtered blood exit the second hemofilter 112 and are returned to the subject.

The circuits of FIGS. 18A and B utilize, for example, central line catheter access (due to the blood flow rates desired to generate beneficial ultrafiltrate flow rates to sustain the nutritional and oxygen needs of the cells) and a multiple pump system with pre- and post-device hemofilters. The actual treatment device portion of the circuit utilizes ultrafiltrate, rather than blood, thus eliminating potential clotting in the cell unit. It also isolates the cells in the device from immunological insult. The therapy circuit can be readily managed in an intensive care unit (ICU) setting.

The premise of this cell-based therapy is that as the ultrafiltrate passes through the cell chamber of the treatment device, systemic small-molecular-weight molecules in the ultrafiltrate, which have been produced due to a disease state, such as a septic disease state, trigger a therapeutic response in the cells within the treatment device, which in turn secrete small-molecular-weight molecules into the passing ultrafiltrate. This processed ultrafiltrate is then refiltered across the hollow fiber membrane of the second hemofilter and is then joined to the filtered blood and returned to the systemic circulation, with the released cell factors providing therapeutic value, for example, to the septic state.

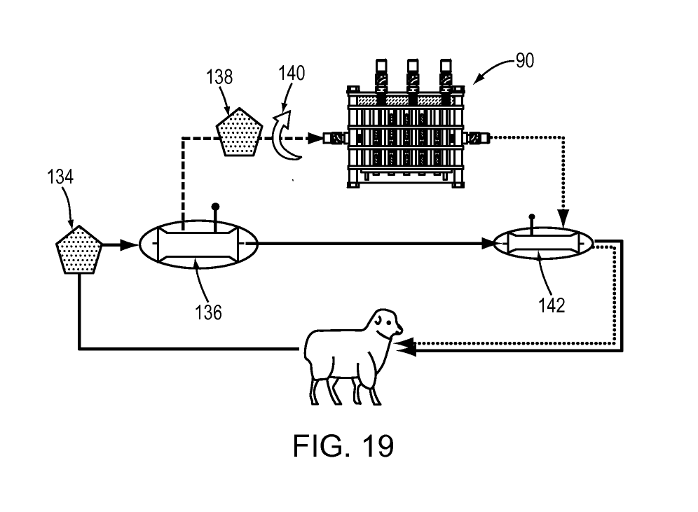

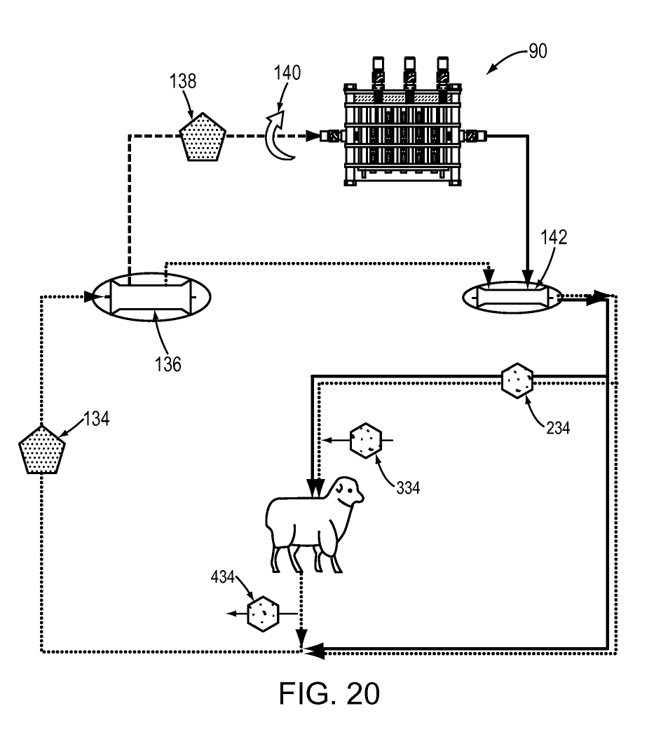

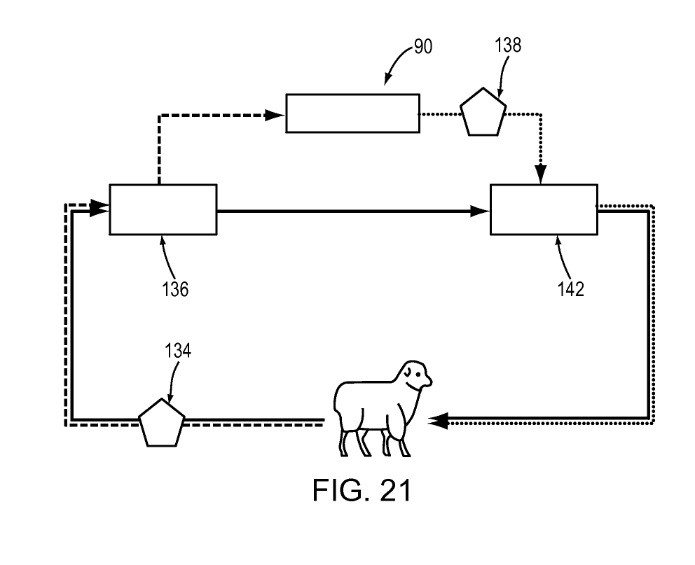

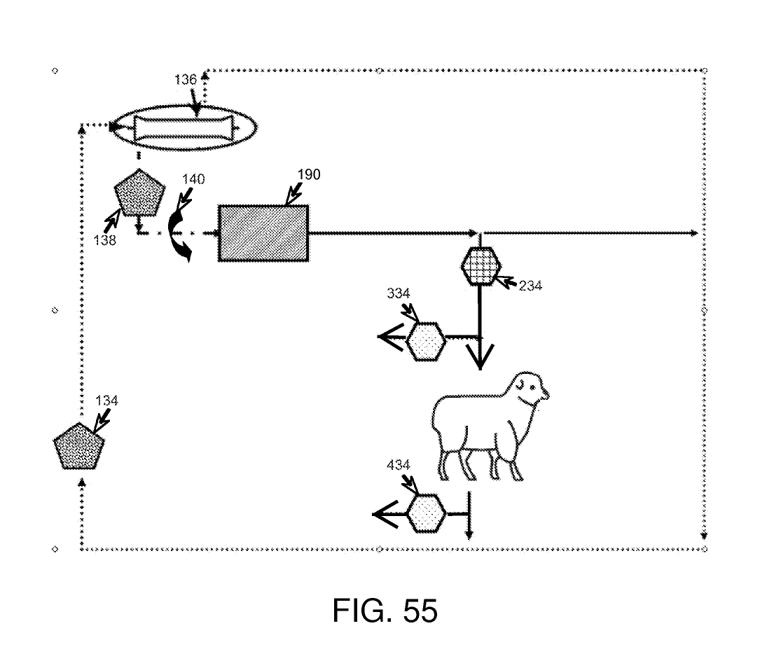

In alternative extracorporeal circuits, such as those shown in FIGS. 19-21 and FIG. 55, the lines may circulate other body fluids, for example, peritoneal fluid that is removed from the peritoneum. Peritoneal fluid is circulated outside the body, where the cells within the treatment device are able to deliver the desired molecules to the fluid. This processed fluid is then reintroduced into the peritoneum. The extracorporeal devices described herein can be used with the extracorporeal circuits of FIGS. 19-21 and FIG. 55. FIGS. 19 and 20 show the device of FIG. 13, but the circuits can be used with other devices according to the invention, such as, for example, the device of FIGS. 15 and 16. Similarly, the circuit of FIG. 21 shows the device of FIG. 13, but it too can be used with other devices according to the invention, such as, for example, the device of FIGS. 15 and 16. Further, the circuit of FIG. 55 shows the device of FIGS. 15 and 16, but it can be used with other devices according to the invention, such as, for example, the device of FIG. 13. In FIGS. 19 and 20, the extracorporeal circuits add peritoneal dialysis fluid to peritoneal fluid. Various compositions of peritoneal dialysis solutions can be used, including, but not limited to, peritoneal dialysate fluid with 1.5% dextrose solution (Deflex®; code 054-20201, Na 132 mEq/L, Ca 3.5 mEq/L, Mg 0.5 mEq/L, Cl 95 mEq/L, Lactate 40 mEq/L, Dextrose 1.5%, available from Fresenius Medical Care North America, Waltham, Mass.), 4.25% dextrose solution (Deflex®; code 054-20204, Na 132 mEq/L, Ca 3.5 mEq/L, Mg 0.5 mEq/L, Cl 95 mEq/L, Lactate 40 mEq/L, Dextrose 4.25%, available from Fresenius Medical Care North America, Waltham, Mass.), or Extraneal™ solution (Na 132 mEq/L, Ca 3.5 mEq/L, Mg 0.5 mEq/L, Cl 96 mEq/L, Lactate 40 mEq/L, Dextrose 0%, Icodextrin 7.5%, available from Baxter International, Inc., Deerfield, Ill.). For human subjects, peritoneal dialysate fluid with 2.5% dextrose solution (Deflex®; code 054-20202, Na 132 mEq/L, Ca 3.5 mEq/L, Mg 0.5 mEq/L, Cl 95 mEq/L, Lactate 40 mEq/L, Dextrose 2.5%, available from Fresenius Medical Care North America, Waltham, Mass.) can be used. The subject's belly is filled with about 1 L to about 3 L of peritoneal dialysis fluid, which is maintained by an additional pump. It is infused at a rate to generally match the reabsorption by the subject. This allows for a volume to be maintained in the belly for recirculation. Although the terminology, peritoneal fluid and peritoneal fluid ultrafiltrate, is used in the description of FIGS. 19-21 and FIG. 55, it should be understood that the circuits can be used with or without the addition of peritoneal dialysis fluid such that peritoneal fluid ultrafiltrate with or without addition of peritoneal dialysis fluid (either before or after ultrafiltration) and/or peritoneal fluid with or without addition of peritoneal dialysis fluid can be used.

In FIG. 19, the peritoneal fluid of a subject (e.g., an animal or a human) is pumped via a first pump 134 through a first dialysis filter 136 having hollow fibers therein defining an ICS and an ECS. The pump can pump at a rate including, but not limited to, of about 25 mL/min to about 200 mL/min The peritoneal fluid passes through the ICS of the dialysis filter 136, and is split into an unfiltered peritoneal fluid fraction and an ultrafiltrate peritoneal fluid fraction. The flow rate of peritoneal fluid ultrafiltrate generated by the first dialysis filter 136 is controlled by a second pump 138 that passes the peritoneal fluid ultrafiltrate from the first dialysis filter 136, through an optional thermal regulator 140 for maintenance of temperature within the device 90 illustrated in FIG. 13, and into the treatment device 90. In the treatment device 90, the peritoneal fluid ultrafiltrate flows across the viable cells on the disks 98, exits the device, and enters a second dialysis filter 142. Alternatively, the thermal regulator 140 need not be used. Upon exiting the second dialysis filter 142, the processed peritoneal fluid ultrafiltrate has been combined with the unfiltered peritoneal fluid, which came directly from the first dialysis filter 136, and is returned to the subject. Peritoneal dialysis fluid is infused via a pump (not shown) at a point before the first pump 134. The extracorporeal circuit of FIG. 21 is similar to the circuit of FIG. 19, except the second pump 138 is placed downstream from the treatment device 90 rather than upstream.

The circuit of FIG. 20 also is similar to that shown in FIG. 19. The peritoneal fluid of a subject (e.g., an animal or a human) is pumped via a first pump 134 through a first dialysis filter 136 having hollow fibers therein defining an ICS and an ECS. The first pump 134 can pump at a rate including, but not limited to, about 5 mL/min to about 200 mL/min The peritoneal fluid is forced from the ICS, across the hollow fibers, to the ECS of the first dialysis filter 136, and all fluid is filtered. Only a portion of the filtered peritoneal fluid, however, moves on to the treatment device 90. The flow rate of peritoneal fluid ultrafiltrate generated by the first dialysis filter 136 is controlled by a second pump 138 that passes some of the peritoneal fluid ultrafiltrate from the first dialysis filter 136, through an optional thermal regulator 140 for maintenance of temperature within the device 90 illustrated in FIG. 13, and into the treatment device 90. In the treatment device 90, the peritoneal fluid ultrafiltrate flows across the viable cells on the disks, exits the device 90, and enters a second dialysis filter 142 with hollow fibers defining an ICS and an ECS. Alternatively, the thermal regulator 140 need not be used. The excess ultrafiltrate that was not delivered to the cells in the treatment device 90 is also passed on to the second dialysis filter 142 and mixed with the processed ultrafiltrate from the treatment device 90. The remixed ultrafiltrate is filtered a second time by being forced across the hollow fibers from the ECS into the ICS of the second dialysis filter 142. Upon exiting the ICS of the second dialysis filter 142, part of the fluid is returned to the subject under the control of another pump 234. The remaining fluid is recirculated to the beginning of the circuit. This circuit may decrease the stress on the abdominal cavity of the subject while still allowing for the flow rates desirable to maintain the circuit and cells. The circuit also allows for ultrafiltration to further protect the subject from bacteria and fibrin produced in the belly of the subject. Because the subject will be resorbing fluid through the abdomen, fresh PD fluid is added to the circuit (at pump 334) to maintain volume in the belly. To ensure balance, at least one pump can optionally be used to pump (at pump 434) some excess fluid to waste.

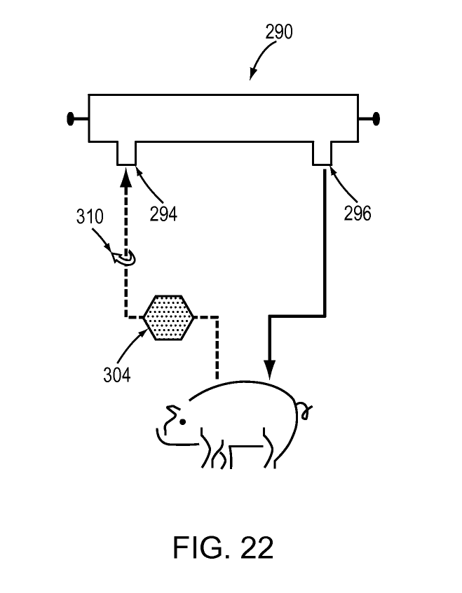

In an alternative extracorporeal circuit, shown, for example, in FIG. 22, the blood from a subject (e.g., an animal or a human) is pumped by a pump 304 (FIG. 22 shows the device of FIGS. 17A and B, but the circuit can be used with other devices according to the invention). The pump 304 passes the blood through a thermal regulator 310 for maintenance of temperature within the treatment device 290, illustrated in FIGS. 17A and B, which has hollow fibers 1152 therein defining an ICS 1140 and an ECS 1142. The blood enters the ECS 1142 of the treatment device 290 through a first port 294, passes by the outside of the hollow fibers 1152, and then exits the device 290 at a second port 290. This processed blood is then returned to the subject. Systemic small-molecular-weight molecules from the blood that have been produced due to the disease state of the subject (e.g., a septic disease state) trigger a therapeutic response of the viable cells, which in turn secrete small-molecular-weight molecules that cross the hollow fiber membrane 1152 and are returned to the system circulation of the subject, providing therapeutic value to the disease state. The circuit of FIG. 22 utilizes, for example, a peripheral catheter for blood access (due to the desirable blood flow rates of less than about 50 mL/min to support the treatment device 290). Peripheral access for this therapy circuit allows it to be managed in a non-ICU-based hospital setting.

Additionally, the lines of the circuit shown in FIG. 22 may circulate other body fluids, for example, peritoneal fluid that is removed from the peritoneum. Peritoneal fluid is circulated outside the body, where the cells within the treatment device is able to deliver the desired molecule(s) to the fluid. This processed fluid is then reintroduced into the peritoneum.

An alternative extracorporeal circuit is shown in FIG. 55. In this circuit design, the peritoneal fluid of the subject (e.g., animal or human) is pumped via a first pump 134 through a dialysis filter 136 having hollow fibers therein defining an ICS and an ECS. The first pump 134 can pump at a rate including, but not limited to, about 5 mL/min to about 200 mL/min The peritoneal fluid to sustain the treatment device 190 is pulled across the hollow fiber membrane from the ICS to the ECS by a second pump 138 and moves through an optional warming device 140 before being delivered to the treatment device 190. The flow rate of peritoneal fluid ultrafiltrate generated by the first dialysis filter 136 is controlled by the second pump 138. In the treatment device 190, the peritoneal fluid ultrafiltrate flows across the viable cells on the disks and exits the device 190. The processed ultrafiltrate continues passage via another pump 234 that controls delivery to the patient. The processed ultrafiltrate that is not delivered to the patient rejoins the remainder of the peritoneal fluid that is not pushed across at the dialysis filter 136, forming a bypass loop. These combined fluids are recirculated to the beginning of the circuit. As with FIGS. 19-21, pump 334 controls the influx of fresh peritoneal dialysis solution to the animal and pump 434 removes processed PD to waste. The absence of the second dialysis filter (e.g., dialysis filter 142 in FIG. 20) allows processed ultrafiltrate to be delivered directly to the patient without interference.

Practice of the invention will be still more fully understood from the following examples, which are presented herein for illustration only and should not be construed as limiting the invention in any way.

### EXAMPLES

### Example 1

Early prototype formulation of miniaturized cell therapy devices are schematized in FIGS. 1 and 8. Of note, this arteriovenous catheter circuit does not require blood pumps for blood flow through the circuit.

Fabrication and in vitro testing of one prototype, shown in FIG. 1, will be assessed. This prototype will be fabricated to contain 1.0×10⁸ renal tubule cells in high density growth within the hollow fibers. Preliminary data suggest that 30 hollow fibers (250 μm×10 cm in length) can maintain 1.0×10⁸ cells in a high-flow situation with adequate oxygenation and nutrient supply in vitro. Initial studies with permanent cell lines have demonstrated that a simpler hollow fiber prototype can maintain this degree of cell density over several weeks. If these initial prototypes are able to maintain cell viability in a cell incubator over 3-5 days, they will be available for efficacy testing in the porcine septic shock model. This experiment is an important proof of concept that renal tubule cells can be placed and maintained in a hollow fiber device with high cell density to provide enough cells in a miniaturized cassette.

Fabrication and in vitro testing of a second prototype with cryopreservation storage capacity. Prototype development of a second cell therapy device is also schematized in FIG. 1. This second device, illustrated in FIGS. 2 and 3, contains a series of disks forming a trabecular structure on which cells attach and grow. The use of individual disks allows for the capability to deliver a range of therapeutic doses taking into account different ages, sizes, and degrees of disease states of the subject. Preliminary data suggest up to 1.0×10⁷ cells can grow on each disk, which is made of a new biomaterial carbon coated with Nb. Ten disks can be placed within a cartridge that acts as a semipermeable membrane to produce plasma ultrafiltrate. This high rate of ultrafiltrate flow provides nutrients and oxygen to the cells on the disks to maintain viability, functionality, and efficacy, while preventing immunologic rejection. Preliminary data have also suggested that the cells on the disk can be cryopreserved and maintain adherence and viability upon thawing and warming to body temperature. Thus, distribution of cell therapy devices will be made simpler with onsite freezer storage, rather than storage in a 37° C. incubator and rapid transport to the clinical care setting from the manufacturing site. When in vitro experiments with this prototype demonstrate maintenance of cell viability over 3-5 days, in vivo testing in the porcine model can proceed.

The extracorporeal device of various embodiments of the present invention can be used with a blood or fluid conduit as illustrated in FIG. 8. The blood from a patient can be through an Aterio Venous (AV) line that could be used in dialysis. Also, venous to veneous (VV) lines can be used. As illustrated, an artery 70 and vein 72 can be used to extract blood and reintroduce blood for an extracorporeal circuit 74. Various suitable connections may be used to increase the diameter of the conduit. The device 30 is illustrated as being attached to the largest conduit 76. The lines may circulate body fluids, for example, peritoneal fluid that is removed from the peritoneum and circulated outside the body where the device is able deliver the desired molecule to the fluid and then reintroduced into the peritoneum.

It is understood, however, that the physicians judgment based upon clinical circumstances should determine on a case by case basis the optimal mode for introducing the device as well as the optimal location for anchoring the device. Such judgments are contemplated to be within the scope of expertise of the skilled physician.

Practice of the invention will be still more fully understood from the following examples, which are presented herein for illustration only and should not be construed as limiting the invention in any way.

Two different device designs are envisioned for the therapeutic delivery system:
1. Cells would be grown on Nb-coated carbon based, disk-shaped scaffolds. These cell-seeded disks would be grown in culture and then cryopreserved. Disks could be shipped frozen to clinical sites were they could be kept frozen until required for cell-based therapy. Thawed disks could be placed in the device housing just prior to therapy. The number of disks used per therapy session (loaded into the holding device) could be varied depending on the cellular dose required.
2. Cells resuspended in a biomatrix 3-D gel would be seeded into the lumen of a hollow fiber bioreactor. Two methods of deployment to the clinical site are to be tested: 1) mature cultured cell loaded bioreactors would be shipped as needed under optimal shipping conditions (to be determined) and then used in the clinical setting. 2) A tube of cryopreserved cells and a tube of pre-mixed liquid biomatrix would be sent to the clinical site. These could be stored until needed. Upon clinical need, cells would be thawed, mixed with biomatrix and syringe loaded into the bioreactor. The cryopreserved tube could contain various cell numbers for dose dependent therapy.

### Cell Seeded Nb-Coated Carbon Based, Disk-Shaped Scaffolds:

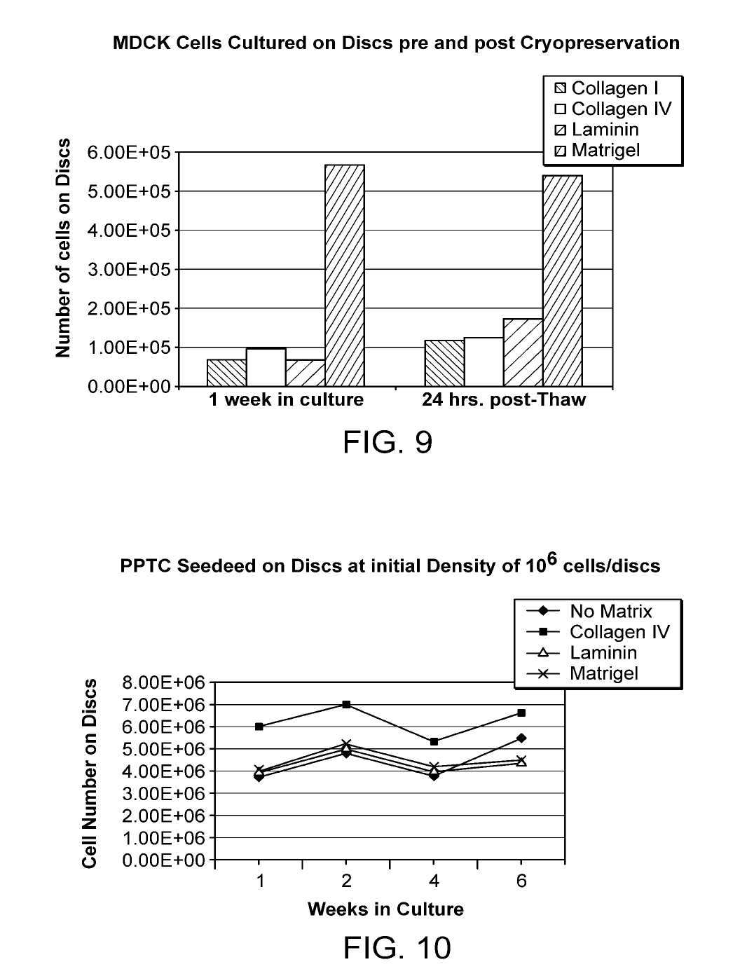

An immortalized renal cell line (MDCK) was seeded unto Nb-coated carbon based disks. Prior to cell seeding, disks were coated with either collagen I, collagen IV, laminin, or matrigel. Seeding densities were 10⁶ cells/disk. The disks were maintained in a spinner culture for 1 week. Cell-seeded disks were then cryopreserved, stored in liquid nitrogen and then thawed, with a non-stir rest period under tissue culture conditions of 2-4 hours post thaw. Disks were then reinstated in spinner culture bottles and assayed for cell density 24 hours post thaw. Lactate production was utilized to determine cell expansion on the disks. MDCK cell densities on disks with various bio-matrix coatings pre and post cryopreservation are depicted in the graph of FIG. 9.

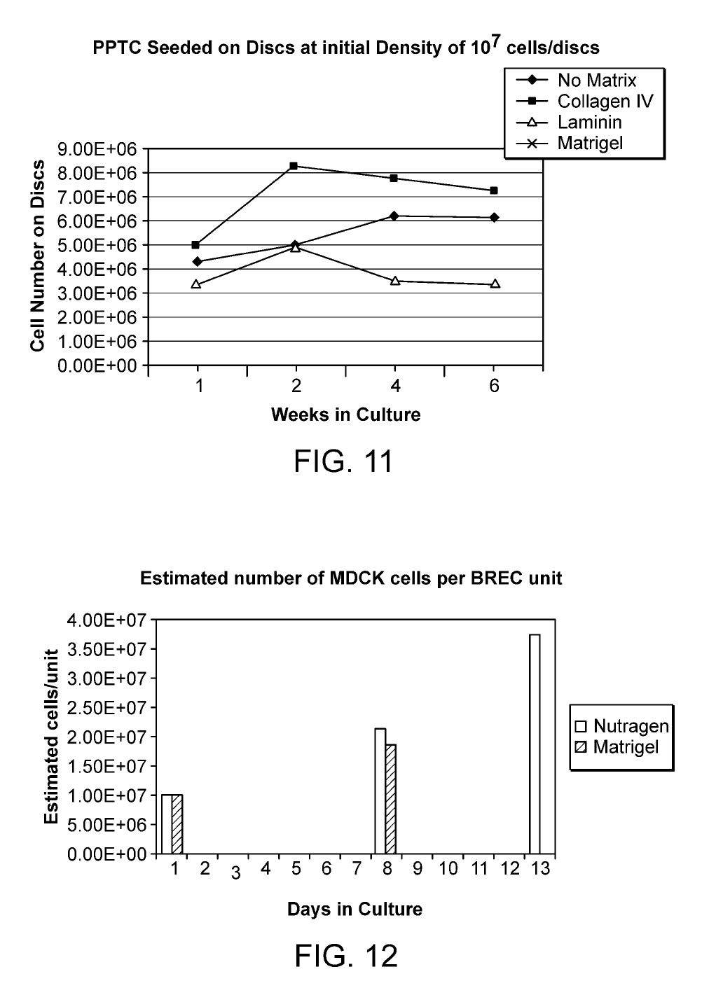

In order to ascertain if primary cells could also be maintained on the Nb-coated carbon based disks, primary porcine renal proximal tubule cells (PPTC) were seeded on disks coated with various bio-matrixes (either no matrix, collagen IV, laminin, or matrigel) at 10⁶ and 10⁷ cells/disk. Cell-seeded disks are currently being maintained in spinner culture. Primary porcine renal tubule cell growth rates on disks, as determined by lactate production, are depicted FIGS. 10 and 11.

### Hollow Fiber Bioreactor Containing Cells Resuspended in a Biomatrix 3-D Gel:

Cells from an immortalized renal cell line (MDCK) resuspended in either 1) nutragen (collagen I (3.12 mg/mL) gel supplemented with laminin (100 μg/mL) and collagen IV (100 μg/mL)) or 2) matrigel were seeded into hollow fiber bioreactors at a density of 2.06×10⁷ cells/mL of gel. Cell loaded bioreactors were maintained in culture for 8 (matrigel) and 13 (nutragen) days, with cell density assessed via lactate production. Results are depicted in the FIG. 12. BREC refers to Bioartificial Renal Epithelial Cell.

### Example 2

### Testing of Cell-Seeded Nb-Coated, Carbon-Based, Disk-Shaped Substrates

This example describes the design of the device illustrated in FIG. 13 (referred to in this Example as the “BRECS-d” or “BRECS”), which is constructed such that the disks can be cryopreserved, allowing for a simplified manufacturing process and ease of clinical storage and deployment. Additionally, in circuits utilizing blood as the treated fluid, although the BRECS-d therapy circuit typically utilizes central line catheter access (due to the blood flow rates desired to generate beneficial ultrafiltrate flow rates to sustain the nutritional and oxygen needs of the cells)) and a multiple pump system with pre- and post-BRECS hemofilters, the actual BRECS-d portion of the circuit utilizes ultrafiltrate rather than blood, thus eliminating potential clotting in the cell unit as well as further isolating the cells in the unit from immunological attack. BRECS-d blood therapy circuits, illustrated, for example, in FIGS. 18A and B, which use the BRECS-d device of FIG. 13, can be readily managed in an ICU setting.

### Background and Significance

Tissue engineering and cell therapy is a new and exciting approach to the treatment of acute and chronic diseases. The potential success of this therapeutic approach lies in the growing appreciation that most disease processes are not due to the lack of a single protein but develop due to alterations in complex interactions of a variety of cell products. Cell therapy is dependent on cell and tissue culture methodologies to expand specific cells to replace important differentiated processes deranged or lost in various disease states. Recent approaches have made progress by placing cells into hollow fiber bioreactors or encapsulating membranes as a means to deliver cell activities to a patient. A reasonable extension of this approach is to add cell therapy to the current successful renal substitution processes of hemodialysis and hemofiltration.

The research outlined herein is a new therapeutic approach based upon the thesis that renal tubule cells play an important immunologic regulatory role in septic shock. Accordingly, the effect of renal tubule cell therapy improves cardiovascular parameters, immunologic activity, and ultimate survival rate of normal, non-uremic large animals in septic shock. The approach is based upon the observation that severe septic shock results in ATN and ARF within hours of bacteremia in a porcine model of septic shock. Thus, ARF develops early in the time course of septic shock, a time frame not appreciated clinically since it takes several days to observe a rise in blood urea nitrogen (BUN) and serum creatinine after the acute insult. Experiments suggest that septic shock induces renal tubule injury, loss of immunologic balance, and cardiovascular collapse with declines in solid organ perfusion. This proposal will develop a device prototype to deliver cell therapy in simpler extracorporeal perfusion systems with greater distribution and storage capabilities for on-site, on-demand availability.

The disorder of ARF, or ATN, may be especially amenable to cell therapy in conjunction with continuous hemofiltration techniques, since acute hemodialysis or hemofiltration has yet to reduce the mortality rate of ATN below 50 percent, despite advances in synthetic materials and extracorporeal circuits. ATN develops predominantly due to the injury and necrosis of renal proximal tubule cells. The early replacement of the functions of these cells during the episode of ATN, which develops concurrently with septic shock, will provide almost full renal replacement therapy in conjunction with hemofiltration. The addition of metabolic activity, such as ammoniagenesis and glutathione reclamation, endocrine activity, such as vitamin D₃ activation, and cytokine homeostasis is contemplated to provide additional physiological replacement activities to change the current natural history of this disease process.

In this regard, the cause of death in patients with acute renal failure is usually the development of SIRS, often secondary to sepsis, with resulting cardiovascular collapse, ischemic damage to vital organs, and MOF. The propensity of patients with ARF to develop SIRS and sepsis suggests that renal function, specifically renal tubule cell function secondary to ATN, plays an important immunomodulatory role in individuals under stress states. The renal tubule cell's roles in glutathione reclamation, glutathione peroxidase synthesis, and activation of vitamin D₃, with its important immunoregulatory functions, are pathways to maintain tissue integrity and host defense under stress conditions. A less recognized role of the kidney, and the renal tubule cells, is its potential immunoregulatory function. The kidney is derived embryologically from dorsal mesoderm, a collection of cells also important in the development of bone marrow stem cells. The maturation of cells responsible for erythropoietin synthesis and activation of 1,25-(OH)₂ vitamin D₃ in the kidney is reflective of this embryonic origin. Phylogenetically, in bony fish and amphibians without lymph systems, the kidney is the major antibody-producing organ. Not surprisingly, mammalian renal proximal tubule cells are immunologically active. They are antigen-presenting cells that possess co-stimulatory molecules and that synthesize and process a variety of inflammatory cytokines.

ARF, and ATN that results in the loss of the kidney's immunoregulatory function, results in a propensity to develop SIRS, sepsis, MOF, and a high risk of death. A rise in sepsis events can be observed from 3% to nearly 60% in patients who develop ARF during the post-op course following open heart surgery. A counterpart to this loss of immunologic function has been observed in chronic renal insufficiency and end-stage renal disease, which are clearly pro-inflammatory states. The degree of inflammation in these patient populations has been highly correlated to mortality rates. The loss of renal tubule cells, rather than loss of filtration and clearance function, may be the cause of this inflammatory dysregulation observed in these patients as well.

In the past decade, a large constellation of data has provided new insights into the inflammatory response that seems to underlie the MOF syndrome. There are now data linking patient outcome to initial plasma levels of tumor necrosis factor (TNF)-a, interleukin (IL)-6, and other proinflammatory cytokines. There is also increasing agreement that organ dysfunction in sepsis arises from oscillating patterns of inflammation from systemic spread of mediators beyond their usual autocrine or paracrine pathways, alternating with immunosuppression either as compensation for or exhaustion of the inflammatory response. This futile and lethal state of affairs has been referred to as “immunologic dissonance.” This paradigm has led to efforts to interrupt this process by targeting individual elements in the cascade or by reducing the overall burden of inflammatory mediators by plasma replacement or adsorption. Other approaches, including plasmapheresis, plasma exchange, hemofiltration, or monoclonal antibodies directed at various components of the inflammatory cascade, have not consistently demonstrated clinically useful interruption of the inflammatory cascade, despite promising in vitro and animal studies.

Bioartificial Renal Tubule Assist Device (RAD)

An extracorporeal device was recently developed that utilized a standard hemofiltration cartridge seeded with approximately 10⁸ renal tubule cells which grow as confluent monolayers along the inner surface of the fibers. The nonbiodegradability and the pore size of the hollow fibers allow the membranes to act as both scaffolds for the cells and as an immunoprotective barrier. In vitro studies of this renal tubule assist device (RAD) have demonstrated that the cells retain differentiated active transport properties, differentiated metabolic activities, and important endocrine processes. See Humes et al., Metabolic Replacement of Kidney Function in Uremic Animals with a Bioartificial Kidney Containing Human Cells, Am. J. Kidney Dis. 39(5):1078-1087 (2002). Additional studies have shown that the RAD, when incorporated in series with a hemofiltration cartridge in an extracorporeal blood perfusion circuit, replaces filtration, transport, metabolic, and endocrine functions of the kidney in acutely uremic dogs. Id. Furthermore, other recent studies have demonstrated that the RAD ameliorates endotoxin shock in acutely uremic animals. RADs consisting of both porcine and human cells have been successfully fabricated and tested.

The bioartificial kidney set-up consists of a filtration device (a conventional hemofilter) followed in series by the tubule RAD unit. Specifically, blood is pumped out of a large animal using a peristaltic pump. The blood then enters the fibers of a conventional hemofilter, where ultrafiltrate is formed and delivered into the lumen of the hollow fibers within the RAD, downstream to the conventional hemofilter. Processed ultrafiltrate exiting the RAD is collected and discarded as urine. The filtered blood exiting the hemofilter enters the RAD through the ECS port and disperses among the fibers of the device. Upon exiting the RAD, the processed blood travels through a third pump and is delivered back to the animal. This additional pump is required to maintain appropriate hydraulic pressures within the RAD. In this regard, the pressures of the blood and ultrafiltrate just before entry into the RAD are monitored. Heparin is delivered continuously into the blood before entering the RAD to diminish clotting within the device. The RAD is oriented horizontally and placed into a temperature-controlled environment. The temperature of the cell compartment of the RAD must be maintained at 37° C. throughout its operation to ensure optimal functionality of the cells. Maintenance of a physiologic temperature is a critical factor in the functionality of the RAD. The unit is able to maintain viability because metabolic substrates and low-molecular-weight growth factors are delivered to the tubule cells from the ultrafiltration fluid and the blood in the ECS. Furthermore, immunoprotection of the cells grown within the hollow fiber is achieved due to the impenetrance of immunoglobulins and immunologically competent cells through the hollow fibers. Rejection of the cells, therefore, does not occur. This arrangement thereby allows the filtrate to enter the internal compartments of the hollow fiber network, lined with confluent monolayers of renal tubule cells for regulated transport and metabolic function.

Improvements to Renal Cell Therapy Devices

The RAD is an extracorporeal device utilizing a standard hemofiltration cartridge containing renal tubule cells grown from adult stem/progenitor cells as confluent monolayers along the inner surface of the fibers. This initial cell therapy device requires large cell numbers for seeding, is labor intensive to generate and maintain and cannot be held in a frozen state for ready deployment at a clinical site. In addition, the RAD is large (12×4-in cylinder) and requires an additional extracorporeal pump circuit to deliver blood and plasma ultrafiltrate to the cell-containing device. The RAD was designed as an add-on to current dialysis treatment in patients with ARF. The clinical use of renal tubule cell therapy for patients with severe sepsis may not require this complex two-cartridge system with two extracorporeal pump systems, since most of these patients are not in ARF. Accordingly, a miniaturized cell therapy device will not require extensive extracorporeal blood pump systems. In addition, a miniaturized device that could be stored at the clinical site for immediate use is desirable to succeed as a commercial product. The current RAD is stored at a central manufacturing facility at 37° C. and must be shipped at 37° C. to the clinical site, delaying treatment and adding to the cost of therapy. Development of a cell device that can be cryopreserved and/or stored at clinical sites is a desirable feature for optimal deployment of such devices. The research described herein will develop a prototype for these more flexible device requirements for easier clinical implementation. As detailed below, this research will develop 1) a miniaturized bioartificial renal cell system (BRECS-d) device that can be manufactured under conditions that allow for cryopreserved cell stocks to be stored at clinical sites for easy deployment and 2) a simplified, bloodless circuit for administration of BRECS-d therapy. These improvements will enable use of the technology for a broader number of indications. Results of the work done with the larger RAD system served as initial proof-of-principle for these miniature BRECS-d devices.

A 10-16 Hour Porcine Gram-negative Model of Septic Shock

Based on severe septic shock results in ATN and ARF within hours of bacteremia in a porcine model of septic shock (as indicated by cessation of urine output and renal histology), the effect of renal tubule cell therapy improves cardiovascular parameters, immunologic activity, and survival rate of normal, non-uremic large animals in septic shock. Thus, ARF develops early in the time course of septic shock, a time frame not appreciated clinically since it takes several days to observe a rise in BUN and serum creatinine after the acute insult. Septic shock induces early renal tubule injury, loss of immunologic balance and cardiovascular collapse with declines in solid organ perfusion.

RAD therapy at the onset of bacteremia (RAD therapy is initiated at the same time that bacteria is infused) in these established animal models “prevents” the rapid deterioration from SIRS. To assess more effectively the efficacy of renal cell therapy to treat sepsis and septic shock, a more reproducible porcine model of septic shock, in which the time to death is greater than 4-6 hours after initiation of sepsis, needs to be developed.

Results

A reproducible model of SIRS in a porcine model of sepsis to test various innovative approaches to treat this clinical disorder has been established and published. See Humes et al., Cell Therapy With a Tissue Engineered Kidney Protects Against the Multi-Organ Consequences of Septic Shock, Critical Care Med. Vol. 31, at 2421-2428 (2003). To improve this animal model, the model was refined to be more consistent and reproducible. The model was also extended to the tissue-specific effects of SIRS after potential therapeutic interventions were attempted. The published model had a wide variability in cardiovascular parameters and time to death (3-7 hours in sham control and 7-12 hours in cell-RAD treatment groups). To improve the reproducibility of this model, volume resuscitation protocol was increased from 100 mL/hr to 150 mL/hr immediately after the crystalloid/colloid bolus infusion at the time of bacteria administration, as detailed below. In addition to this improved resuscitation protocol, the tissue-specific consequences of sepsis with or without RAD therapy were evaluated with bronchoalveolar lavage (BAL) to better understand the immunoregulatory role of renal tubule cell therapy. BAL specimens were used to assess pulmonary microvascular damage and inflammation in response to SIRS. Data detailed below demonstrated that renal cell therapy was associated with less protein leak from damaged blood vessels and less inflammation in BAL fluid samples, as well as improvement of other cardiovascular effects of SIRS.

Pre-Clinical Large-Animal Model

The improved porcine model of septic shock was used to preliminarily assess the multiorgan effects of RAD intervention. The following data are derived from a total of 14 animals: 7 sham controls and 7 RADs (second cartridge containing porcine renal epithelial cells).

Cardiovascular Parameters

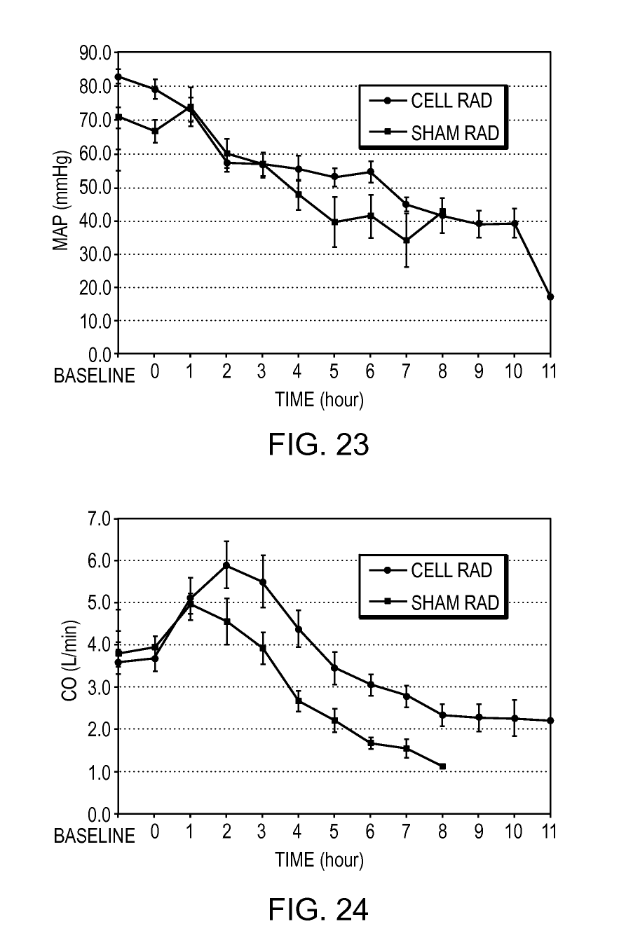

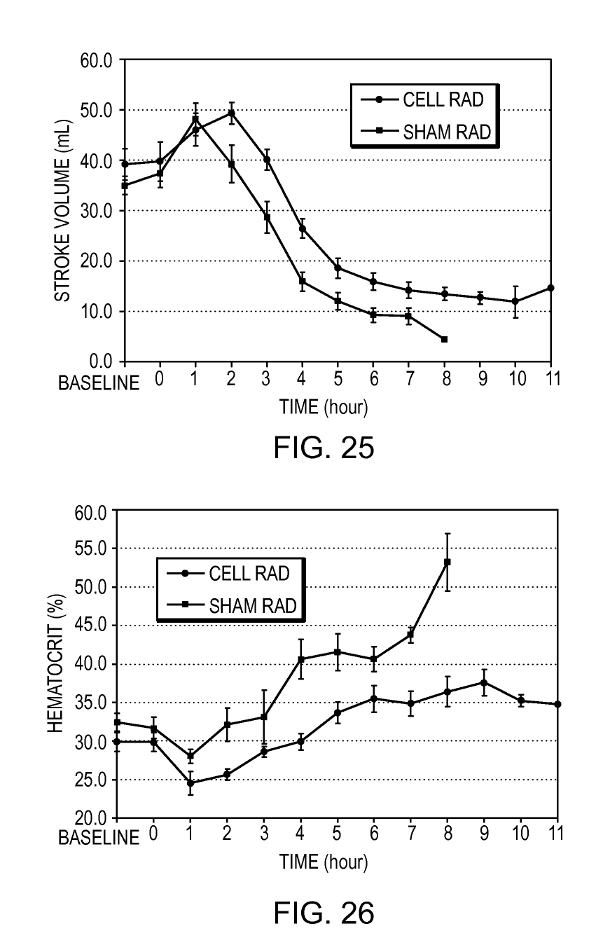

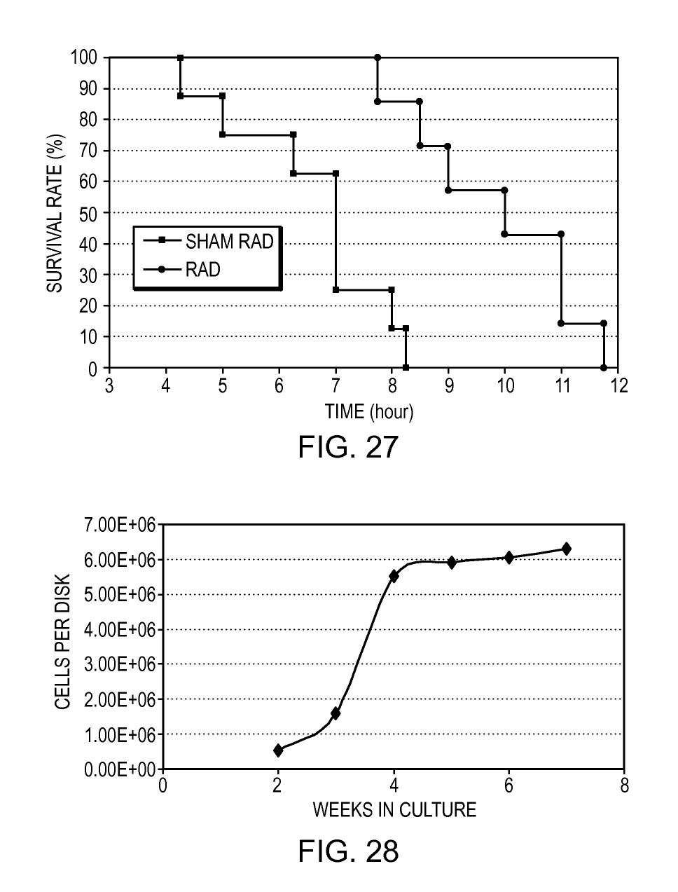

As demonstrated in FIG. 23, the administration of bacteria into the peritoneal cavity induced a rapid, profound, and eventually fatal decline in mean arterial pressure (MAP) in all groups. The cardiac outputs (CO) are detailed for each group in FIG. 24. The CO was substantially higher in the RAD group compared to the sham group. A similar difference among the groups was observed in stroke volume as well (FIG. 25). As an approximate measure of systemic capillary leak induced with this septic course, the changes in hematocrit are displayed in FIG. 26. As shown, the sham controls have a higher rate of increase with time, reflective of larger rates of volume loss from the intravascular compartment, compared to the RAD group. These changes were associated with a substantial survival advantage in the RAD group compared to the sham control (FIG. 27). The average survival times were 6.6 hours±0.5 and 9.9 hours±0.6 for control and RAD groups, respectively.

These data with this improved porcine model confirm that the RAD improved cardiac performance, renal blood flow (data not shown), and survival times compared to sham controls.

To investigate the effect of various therapeutic interventions in this model of SIRS, BAL fluid was obtained at the time of death and evaluated for protein content as a parameter of microvascular damage, various inflammatory cytokines and the absolute number of polymorphonuclear cells (PMNs). As summarized in Table 1 below, data suggested that RAD treatment resulted in less vascular damage and protein leak and less inflammatory cytokine release in the early phase of pulmonary involvement in SIRS. Levels of IL-6, IL-8 and TNF-α were lower in the treatment interventions versus sham controls. Levels of IL-1 and IL-10 (not shown due to no detectable levels in each group) were not different. Absolute neutrophil counts in the sham controls were above 1000 cells/mL, and the RAD group trended lower, although the n=2 in each group does not allow for statistical analysis.

TABLE 1
Protein and cytokine levels in bronchoalveolar lavage (BAL) fluid from
pigs with septic shock.

| | Protein | TNF-α | IL-1 | IL-6 | IL-8 |
|---|---|---|---|---|---|
| | (μg/mL) | (pg/mL) | (pg/mL) | (pg/mL) | (pg/mL) |
| Sham control | 143 ± 11 | 21 ± 1 | 18 ± 2 | 63 ± 14 | 126 ± 42 |
| (n = 6) | | | | | |
| RAD (n = 3) | 78 ± 10 | 18 ± 5 | 18 ± 5 | 32 ± 6 | 33 ± 10 |

Note:
Mean ± SE. BAL performed at time of death.

BAL sampling was initiated for a subset of animals to assess the utility of this analysis. With these encouraging results, the next steps will incorporate this as a routine inflammatory index at 3-4 hours post-bacteria infusion. Data for plasma levels of various cytokines also showed substantive effects in the RAD treatment group compared to sham controls. At 6 to 8 hours post-sepsis induction, plasma levels for IL-10 were 59±13 and 49±16 (p=0.03); IL-8, 602±550 and 168±165 (p=0.01); and IL-6, 8.3±3.4 and 9.0±2.6 (p>0.05) for the RAD (n=7) and sham controls (n=7), respectively. The higher levels of IL-8 may reflect the effect of sepsis in increasing IL-8 release from renal epithelial cells.

In summary, a more reproducible porcine model of sepsis was developed with longer survival times to compare therapeutic efficacy of new device formulations, which will be the focus of the next research steps. In addition, tissue-specific inflammatory effects of therapy with BAL fluid analysis were incorporated into this model system.

### Test the Efficacy of the RAD in the Reversal of Septic Shock

This research will develop further preclinical data to demonstrate that cell therapy with a RAD can also reverse and improve cardiovascular performance, cytokine balance and survival times in the porcine model of E. Coli-induced septic shock discussed above. As opposed to the established “prevention” model of septic shock described above, which introduces sepsis and RAD therapy simultaneously, this proposed model will more closely simulate the clinical setting. Septic shock will be induced 2-4 hours prior to initiation of RAD therapy. This model will assess the ability of the RAD to reverse the process of septic shock, as measured by increased survival time, hemodynamic stability and reestablishment of cytokine balance.

Results

With the development of this improved porcine model, cell therapy interventions were introduced 2-4 hours after induction of sepsis to evaluate the efficacy of the RAD in reversing the progression of septic shock. Several animals were tested with the introduction of the RAD into the extracorporeal circuit 2-4 hours into sepsis. The introduction of the RAD and its accompanying blood lines resulted in such a drastic after-load reduction maneuver that all animals died within one hour of device placement, despite large volume resuscitation efforts with both acellular and cellular devices. Thus, this acute model system was not adequate to assess the potential for reversibility of septic shock progression. The results of ongoing clinical trials with human cell RADs, however, have presented data supportive of reversing the progression of sepsis in patients with ARF and MOF.

### A Simplified Extracorporeal Circuit for RAD Therapy

Current extracorporeal treatment with the RAD requires blood to be circulated into the ECS of the RAD while ultrafiltrate (UF) from the synthetic hemofilter is delivered to the luminal spaces in which the cells reside in the RAD, duplicating the structural and functional anatomy of the nephron. Blood perfusion in the ECS makes the treatment with the RAD more complex and, perhaps, less safe (due to clotting and increased patient blood volume required by RAD circuit). Utilizing UF delivery alone to the RAD may provide sufficient pathophysiologic signals to the renal tubule cells to exert their immunoregulating effects during SIRS. A series of experiments were established to evaluate whether the biochemical effects of RAD therapy and potential amelioration of the natural history of SIRS in this porcine model can be accomplished with removal of blood perfusion within the RAD circuit.

Results

Work focused on the in vitro development, followed by a preliminary proof-of-concept pre-clinical animal testing, of a miniaturized BRECS to replace renal metabolic and hormonal processes. The successful employment of the BRECS unit will enable use of this technology for a broader number of indications, including utilization of a blood-free delivery system. Results of the work done above with the larger renal assist device (RAD) serve as initial proof-of-principle for the miniature BRECS unit. A BRECS unit that could be stored at the clinical site for immediate use will improve its ability to be used in a clinical setting. Development of a cell device that can be cryopreserved and stored at clinical sites is desirable clinically. The most advanced design of these BRECS prototypes (the carbon disk-based BRECS-d) was then tested in 2 animals, under the established porcine model of sepsis, utilizing an extracorporeal system that incorporated ultrafiltrate delivery, rather than blood, to the BRECS-d unit. The data below detail 1) the in vitro results that show the BREC-d design to allow for a robust cell system that can be cryopreserved and thawed with no detrimental effects on cell viability and 2) the incorporation of the BRECS-d unit in the septic animal model utilizing ultrafiltrate delivery, rather than blood, showing an improvement in survival time over both sham- and RAD-treated animals.

### BRECS-d In Vitro Data

PPRECs were seeded on Nb-coated carbon-based disks at a density of 10⁶ cells/disk. Prior to cell seeding, disks were coated with collagen IV. Preliminary studies determined that collagen IV—coated disks displayed superior cell growth as compared to disks coated with collagen I, laminin, or matrigel. The disks were maintained in a spinner culture for up to 7 weeks. After the initial post-seeding expansion, cell number, as determined by lactate production, remained stable over the 7-week period (FIG. 28).

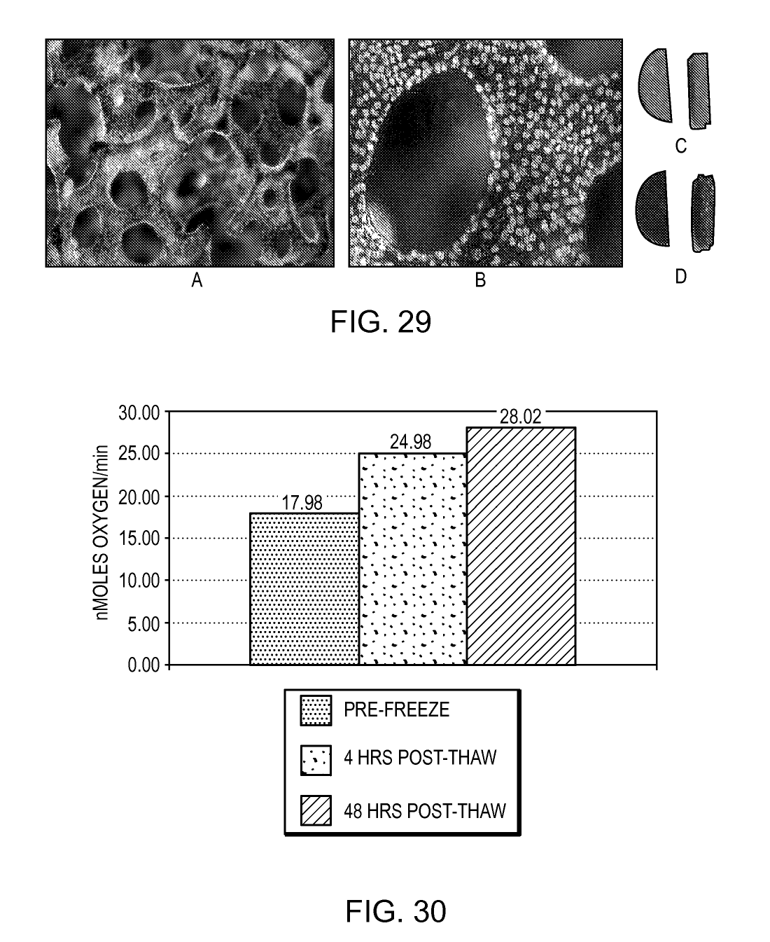

Cell growth coverage on the disks was assessed by both staining with crystal violet and DAPI. FIGS. 29A-D show the disks to be covered uniformly, as assessed by these two staining methods. FIG. 29A shows a cell-seeded carbon disk after several weeks in Starwheel culture stained with DAPI, a nuclear stain. FIG. 29B is a close-up view of a section of FIG. 29A. FIGS. 29C and D show a collagen IV-coated sham carbon disk and a cell-seeded carbon disk, respectively, after staining with crystal violet. Cells are readily visualized via the dark staining on the cell disk as compared to no staining on the sham disk.

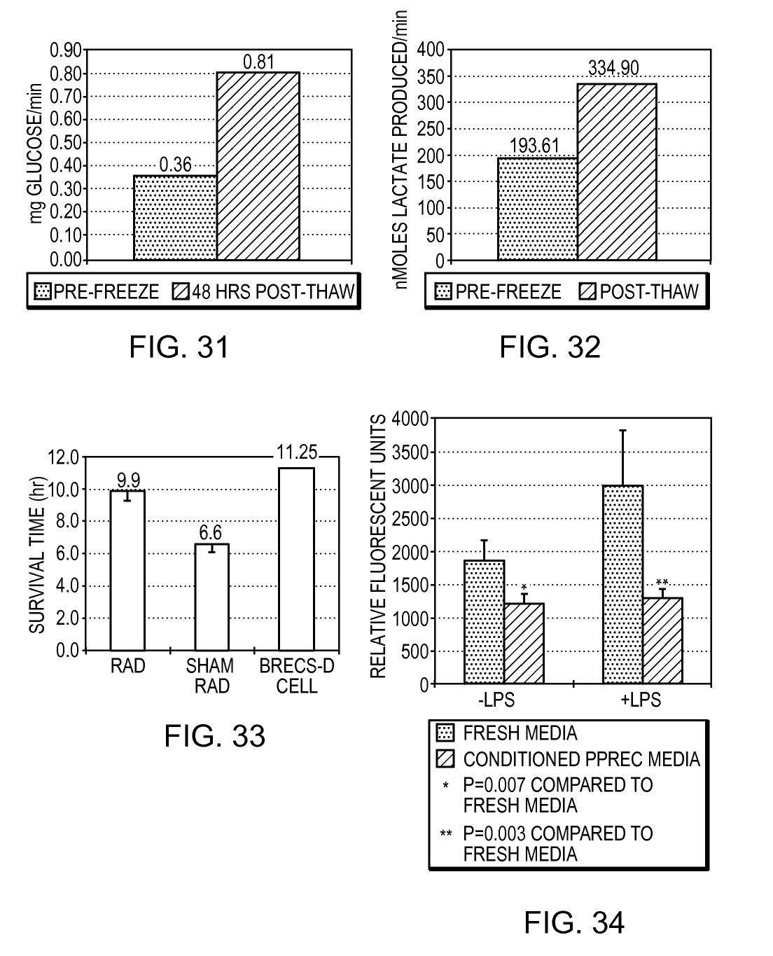

To determine the feasibility of cryopreservation of the cell-seeded disks, the following study was performed. PPRECs were seeded on Nb-coated carbon-based disks, pre-coated with collagen IV, at a cell density of 10⁶ cells/disk. The disks were maintained in a Starwheel spinner culture for two weeks. The Starwheel system was designed so that four disks were held by a tuning fork (i.e., a scaffold) in a vertical position with up to eight scaffolds per Starwheel flask. After two weeks, five scaffolds, each containing four disks, were placed in a BRECS-d unit for pre-cryopreservation oxygen and glucose consumption and lactate production rate determination. The cell-seeded disks were then cryopreserved in groups consisting of four disks per scaffold system, stored in liquid nitrogen for 48-72 hours, and then thawed for post-cryopreservation oxygen and glucose consumption rate assessment. The 4-disk groups were quick thawed at 37° C., and the five scaffold systems were placed in the BRECS-d unit illustrated in FIG. 13. The BRECS-d unit was perfused with media at increasing flow rates over a 2-hour period. The post BRECS-d perfusate was collected for cell counts to determine cell attachment post-cryopreservation. The number of released cells in the post BRECS-d perfusate was insignificant as determined by hemocytometer (data not shown). FIGS. 30, 31, and 32 depict pre- and post-cryopreservation oxygen consumption rates, glucose consumption rates, and lactate production rates, respectively. Insofar as oxygen rates were not decreased, it is contemplated that freeze/thawing does not negatively impact cell viability of frozen cells.

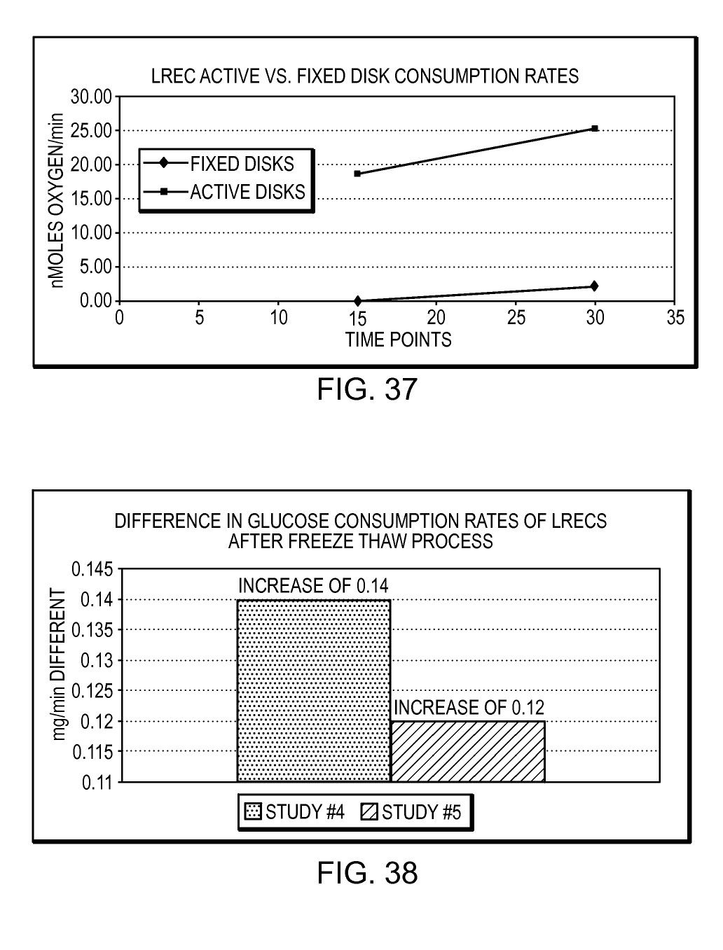

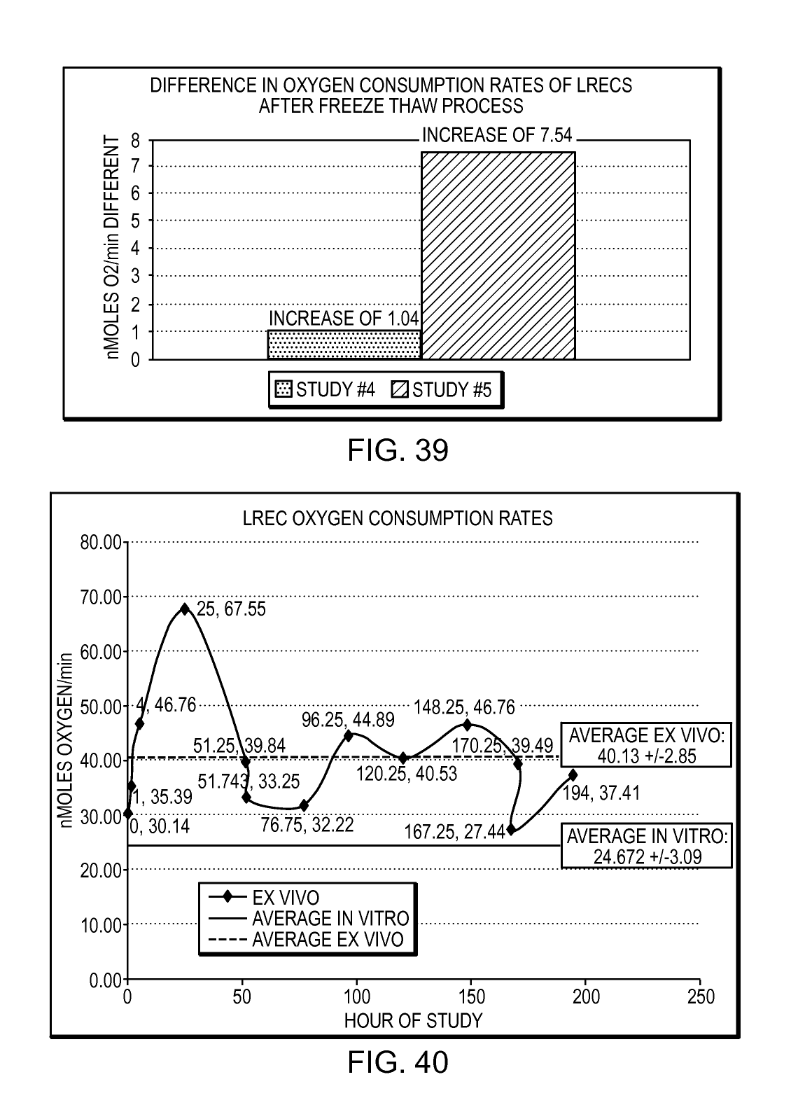

Apart from these results, further in vitro testing of the BRECS-d unit, illustrated in FIG. 13, is shown in FIGS. 37-39. FIG. 37 shows a comparison of lamb renal epithelial cell (LREC) active disk consumption rates versus LREC fixed disk consumption rates over time. FIG. 38 shows the difference in glucose consumption rates of LRECs after a freeze thaw process. FIG. 39 illustrates the difference in oxygen consumption rates of LRECs after a freeze thaw process. Study 4 and Study 5 of FIGS. 38-39 were two different sets of LRECs grown on disks that underwent the cryopreservation process. These two studies demonstrated maintenance of cell viability when seeded on disks after thawing (i.e., post-cryopreservation). The results compare the measured consumption rates of the cells pre- and post-freeze to show that the cells are viable after undergoing such a strenuous process. These results indicate that the cells can be frozen on disks and then be viable and metabolically active after being thawed.

### BRECS-d Preliminary Pre-Clinical Animal Studies

Upon the above promising in vitro studies shown in FIGS. 30-32, two PPREC-seeded BRECS-d units were prepared for testing in the large-animal model system previously discussed. The BRECS-d circuits displayed in FIGS. 18A and B, which were previously described, were both tested in one animal each, using the BRECS-d device shown in FIG. 13.

The study results obtained from these two animals treated with the device shown in FIG. 13 in the circuits of FIGS. 18A and B, displayed similar, if not improved, therapeutic efficacy as the RAD-treated group of animals compared to sham-treated when evaluated for hemodynamic parameters (mean arterial pressure, cardiac output, and stroke volume), hematocrit, and systemic cytokines (data not shown). This improved outcome was most apparent in the survival time. For one of the two BRECS-d-treated animals, the study was stopped due to achieving the study end point of 12 hours. Survival times for animals treated with RAD, sham, and BRECS-d are displayed in FIG. 33. The BRECS-d result shown in FIG. 33 is a combination of data from one animal treated using the circuit of FIG. 18A and one animal treated using the circuit of FIG. 18B.

### Effect of PPREC-Conditioned Media on Activated Leukocytes

The effect of PPRECs on normal porcine leukocyte activation was assessed. These studies isolated leukocytes from heparinized blood utilizing polymorph solution (per manufacturer's recommendation), followed by a 30-minute incubation period with or without LPS. During this 30-minute period, the leukocytes were incubated in fresh media or PPREC-conditioned media (exposed to PPRECs for 24 hours prior to removal for leukocyte incubation). Intracellular oxidative capacity was measured at the end of the incubation period by addition of 5 μM 2′,7′-dichlorofluorescein (DCFH)-diacetate. The PPREC-conditioned media significantly decreased leukocyte activation, as determined by oxidative burst, under both non-LPS and LPS conditions compared to the fresh media condition. The results are depicted in FIG. 34 with DCHF expressed as relative fluorescent units (RFUs). This result suggests that renal epithelial cells secrete a soluble substance in the conditioned media to inhibit leukocyte activation, and it supports the developing hypothesis that renal epithelial cells produce immunomodulatory compounds as a mechanism of action accounting for their efficacy in tissue injury.

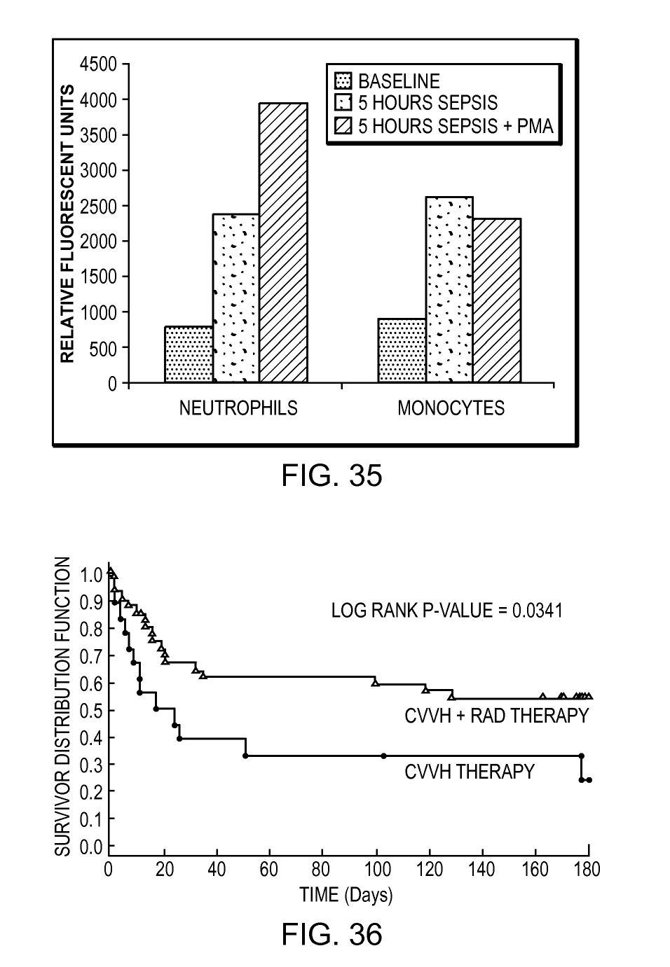

Although the above-described leukocyte oxidative burst assay generated preliminary data that could be used to show the potential for assessing differences in the activated state of systemic leukocytes, this assay can be cumbersome and dependent on the isolation methods employed to obtain the leukocytes to be used in this assay. Variability was found due to the isolation process itself activating the leukocytes. An alternative method which could be used as an indicator of leukocyte activation has now been developed. The following protocol will be used for assessing the level of neutrophil, monocyte, and eosinophil activation in porcine peripheral blood. This approach measures the level of CD11b expression on the cell surface of each leukocyte population as an indicator of the “activation” level attained by the population. Neutrophils and to a lesser extent monocytes, and to an even lesser extent eosinophils, mobilize intracellular stores of CD11b to the cell surface as they become activated. The protocol calls for measuring the level of leukocyte surface expression by staining freshly isolated whole blood with a FITC-conjugated anti-porcine CD11b antibody. Red cells are then lysed and the leukocytes fixed using Becton-Dickenson's “Facs lyse” solution. This serves a three-fold purpose: it fixes the antibody-stained leukocytes for delayed flow cytometry analysis, it destroys the red blood cells, eliminating them from the analysis, and it separates the two granulocyte populations (neutrophils and eosinophils) from each other on the flow cytometer's forward and side scatter plots. Cells are then collected by centrifugation and resuspended for flow cytometry analysis. The data depicting the ability to assess the level of activation in various leukocyte populations is shown in FIG. 35. Leukocytes were processed from systemic blood at baseline and 5 hours post bacterial infusion from an acellular (sham) treated pig. The blood was then either incubated immediately with a FITC-conjugated anti-porcine CD11b antibody or after a 30 minute exposure to PMA. The PMA condition acts as a positive control for maximal stimulation of leukocytes. The antibody/blood mixture was then fixed with a Becton-Dickenson's “Facs lyse” solution and the leukocyte population was analyzed for activated neutrophils, monocytes and eosinophils. The intent of this data was to assess the assay ability to discern between various leukocyte cell populations and be a sensitive index of leukocyte activation. From the data generated, both of these criteria were achieved.

This assay will be used in two ways. First, the effect of cell therapy compared to sham controls on leukocyte activation will be assessed. Blood will be collected and leukocytes isolated at baseline (prior to bacteria infusion) and then again at 6 hours post-bacteria infusion. The intensity of fluorescent signal of FITC-conjugated anti-porcine CD11b antibody bound to the leukocyte surface will be analyzed by flow as an indicator of leukocyte activation and compared between sham- and cell-treated groups. The effect of an HREC-seeded BRECS-d device on leukocyte activation in an ovine anephric model is described in Example 7. Second, conditioned media will be collected from the BRECS-d units and incubated with normal, non-septic isolated porcine leukocytes under non-LPS and LPS exposure conditions. Anti-porcine CD11b antibody staining of the leukocytes will be assessed and used as a biomarker index of the BRECS unit's ability to inhibit the activation of leukocytes. It is contemplated that the BRECS unit will function similar to the kidney, by secreting various cytokines/molecules to mediate the activation of the leukocytes.

### Clinical Evaluation of Efficacy of Renal Cell Therapy

After encouraging pre-clinical animal data, including the use of the porcine SIRS model described previously, the FDA approved the initiation of a Phase I/II clinical trial to evaluate the safety and efficacy of this renal cell therapy utilizing the RAD on 10 critically ill patients with ARF and multiorgan failure receiving CVVH. See Humes et al., Initial Clinical Results of the Bioartificial Kidney Containing Human Cells in ICU Patients with Acute Renal Failure, Kidney Int'l 66(4):1578-1588 (2004). The predicted hospital mortality rates for these patients averaged greater than 85%. The devices used in this study were seeded with human renal proximal tubule cells isolated from kidneys donated for cadaveric transplantation but found to be unsuitable for transplantation due to anatomic or fibrotic defects. The results of this clinical trial demonstrated that the experimental treatment could be delivered safely under study protocol guidelines for up to 24 hours when used in conjunction with CVVH. The clinical data indicated that the RAD exhibited and maintained viability, durability, and functionality in this clinical setting. Cardiovascular stability of the patients was maintained, and increased native kidney function, as determined by elevated urine outputs, temporally correlated with RAD treatment. The device also demonstrated differentiated metabolic and endocrinologic activity. All but one treated patient with more than a 3-day follow-up showed improvement, as assessed by acute physiologic scores. Six of the 10 treated patients survived past 28-days with kidney function recovery, although mortality rates predicted for these 10 patients using the acute physiology, age, chronic health evaluation (APACHE) 3 scoring system were on average 85 percent. Plasma cytokine levels suggests that RAD therapy produces dynamic and individualized responses in patients depending on their unique pathophysiologic conditions.

These favorable Phase I/II trial results led to a subsequent FDA-approved, randomized, controlled, Phase II investigation at 12 clinical sites to determine whether this cell therapy approach alters patient mortality. This Phase II study involved 58 patients, of whom 40 were randomized to RAD therapy and 18 made up a control group with comparable demographics and severity of illness by SOFA scores. Of importance, the incidence of sepsis in this clinical trial was 73% in the RAD treated group and 67% in the control group. The efficacy of the RAD in patients with sepsis demonstrates the ability of renal cell therapy to reverse sepsis in the clinical setting. The early results were as compelling as the Phase I/II results. Renal cell therapy improved the 28-day mortality rate from 61% in the conventional hemofiltration-treated control group to 34% in the RAD-treated group. This survival impact continued through the 90- and 180-day follow-up periods (p<0.04), with the Cox proportional hazard ratio indicating that the risk of death was 50% of that observed in the continuous venovenous hemofiltration (CVVH) therapy group (FIG. 36).

As shown above, the BRECS-d design allows for the device to be both miniaturized and cryopreserved, thus eliminating the shortcomings of the original RAD design, such as needing to maintain the RAD in culture prior to clinical use, which demanded significant hands-in technical time. The technology is a bioengineered device that utilizes renal cells in the treatment of diseases, such as SIRS and septic shock. The target treatment population includes patients with SIRS and septic shock. Based on the in vitro testing results shown in FIGS. 37 through 39, further optimization of the BRECS-d design will include the following: (1) implementation of an oxygen probe/sensor system; (2) inclusion of a filtration system design that may use miniaturized pumps or eliminate immunoprotection methods; and (3) optimization of seeding methods/cell density by way of dynamic seeding and multiple seeding. A compressed BRECS-d unit is contemplated that would be wearable for therapy. This would be useful in clinical applications.

### Example 3

### Development and Testing of a Fully-Freezable or Cryopreservable BRECS-d Unit

As described above, the BRECS-d device of FIGS. 13 and 14, with its cryopreservable and thawable disks, is useful to facilitate easy manufacturing and quick development at a treatment center. However, the entire device might be cryopreserved, rather than just the disks, to further facilitate manufacturing and deployment. An example of such a fully-freezable device is shown in FIGS. 15 and 16. Accordingly, this example describes the design considerations and testing for development of a fully-freezable embodiment of the treatment devices of the invention, such as that shown in FIGS. 15 and 16. As used in this example, “BRECS” and “BRECS-d” refer to a fully-freezable treatment device, such as that shown in FIGS. 15 and 16.

The goal is to develop a prototype design to allow the entire BRECS-d unit to be cryopreserved. Additionally, the BRECS-d system will continue to be tested in vitro to assess cryopreserved shelf life, establish protocols for manufacturing, transportation and clinical deployment. The prototype development will determine the robustness of the unit design within the perceived use/lifecycle environment. The lifecycle process consists of the following: 1) injection molding of the unit, followed by sterilization and packaging, 2) sterile loading of the cell disk tuning fork cassettes into the unit, 3) sterile filling of the unit with freezing medium, and 4) sealing the cell holding unit for storage. The unit will then be stored in a sterile cryo-compatible bag filled with the same freezing medium. Once sealed, pressure ports will be opened to allow for fluid expansion, and the unit will begin a temperature step-down process, in reasonable degree increments that allow for immersion in liquid nitrogen for maintenance of maximum cell viability over an indefinite length of storage time. At point of use, the bagged unit is removed from its liquid nitrogen storage and placed into a bath of 37° C. water for thawing. Once the bagged unit is temperature stabilized, the pressure ports will be closed to maintain sterility as the unit is removed from the bag, dried, aseptically wiped, and connected to the ancillary circuit for patient use.

The resins being contemplated for injection molding are approved for biomedical use and have data showing compatibility with EtO and gamma radiation sterilization. Additionally, these materials have been used in cryo-storage tubes, but data have not been found showing performance at extended extreme low temperatures under the exact conditions of the envisioned prototype. The issues of thermal shock, stress cracking, and impact strength will be monitored, as well as differences between the coefficient of thermal expansion of the threaded fasteners and gasket material. The gaskets and fasteners are rated for the sterilization and low temperatures. The development process will address these parameters and provide data showing the robustness of the molded device's performance within its proposed environment.

Testing on the device will include the following.
1. Establish a shelf life with respect to cryopreservation time. BRECS-d units will be cryopreserved for increasing times to determine if there are limits to the cryo-storage time. Units will be tested at 1 week, 1 month, 3 months, 6 months, and 12 months post cryopreservation. In vitro test parameters will include 1) lactate production, 2) oxygen and glucose consumption, 3) cell release upon re-thaw and perfusion, 4) conditioned media ability to inhibit leukocyte activation, and 5) IL-8 secretion under baseline and LPS stimulated conditions. Each unit will have pre-cryopreservation baseline parameters taken for direct comparison. Five BRECS-d units will be cryopreserved for each of the first 4 time point with 6 BRECS-d units being used for the 1 year time point. It is contemplated that the maintenance of cell viability post-freeze will be within an acceptable standard deviation and that the integrity of the BRECS materials will be retained post-thaw. Further testing will test the longevity of the device post-freeze.
2. Develop protocols for use in manufacturing, transportation, and clinical deployment. Manufacturing standard operating procedures will be developed using human renal cells. Packaging for dry ice shipment will be determined and practice shipping runs will be held utilizing actual shipping service providers. Kits will be developed for use at the clinical site for the thawing, rinsing and circuit integration of the BRECS-d unit. Thirty units will be used to carry out these planned studies (10 each for each of the protocol areas: manufacturing, transportation, and clinical deployment).
3. Release criteria will be established. Also, parameters will be established for optimal conditions for the expansion of PPRECs on the carbon disks. Cell seeding density and protocol, collagen IV—coating concentration and method, various stir rates while in culture will be assessed and cryopreservation freezing media and protocols will all be reviewed and assessed for optimization. Forty units will be used to carry out these planned studies (10 each for 2 release criteria, 10 for assessment of the cryopreservation protocols, which after the optimal protocol is determined, it will be employed in the shelf life studies and 10 for the optimization of culture parameters).

Therefore, a total of 96 BRECS-d units (26+30+40) will be tested in the in vitro study plan. The above studies will use 8 PPREC isolations in order to have primary cells from multiple animals.

Methods

The Starwheel carbon-based disk cultures will be established utilizing the following protocol: 8×10⁷ PPRECs per 10 mL media will be perfused under recirculating conditions into a flow through chamber containing collagen IV—coated disks (0.5 μg/cm²) to allow for the PPRECs to optimally infiltrate the center of the carbon disks. Disks are incubated at 37° C. for 3-4 h before being placed in the Starwheel Dynamatrix cell growth system. Cells are spun at 2 rpm and in 175 mL of media. Media is changed 3 times weekly by aspirating old media through side arm and adding new media. Side arm caps are loosened a quarter turn to allow for gas exchange in incubator. Disks are typically used after 2-5 weeks of culture. For initiation of the BRECS-d culture, Starwheel disk tuning forks, containing 4 cell-seeded carbon disks each, are sterilely transferred to the BRECS-d system. The BRECS-d unit will then either be connected to the in vitro perfusion system for culture and in vitro studies or prepared for cryopreservation. Tuning fork cryopreservation protocol will be followed for the initial BRECS-d cryopreservation protocols. The BRECS-d chamber will be filled sterilely with a freezing medium; care will be taken so that the fill level covers all disks but still allows for expansion during the freezing process. The unit will be placed in a sterile bag designed for cryopreservation and the bag will then be filled with freezing medium and sealed such that the BRECS-d unit ports can be manipulated and caps opened for release of any pressure build-up that could develop during the cryopreservation process. After opening of the ports, the BRECS-d cryo-system will then be reduced in temperature in a step-down process until transfer to liquid nitrogen. At the time of use, the BRECS-d cryo-system will be removed from liquid nitrogen storage and placed in a 37° C. waterbath for rapid thaw. The unit will then have the ports closed while still in the cryo-bag and will then be removed once port closure and device integrity have been visually confirmed. The BRECS-d will be sterilely connected to a perfusion circuit, utilizing techniques similar to those used for peritoneal dialysis catheter connection. The perfusion rate will be slowly increased until a desirable rate to sustain the nutritional and oxygen needs of the cells is achieved. Lactate will be measured using a commercially available kit (BioVision, Inc., Mountain View, Calif.). Glucose levels will be measured via a glucometer. Oxygen consumption will be measured online with, for example, a multi-frequency phase fluorometer that utilizes a fiber optic reflection oxygen probe and a ruthenium based or porphyrin complex sensor that works by fluorescence quenching of the complex to measure the partial pressure of dissolved or gaseous oxygen. Cell release will be assessed via manual hemocytometer cell counts and leukocyte activation via the DCFH assay described above.

Contemplated Results

A BRECS-d prototype will be designed and fabricated that can be cryopreserved and thawed. This design will allow for a BRECS-d unit that has therapeutic value. A shelf life of up to 1 year will be determined using established techniques for the cryopreservation of PPREC. These cells have been thawed after several years of cryopreservation with no noticeable negative effect.

### Assessment of the Therapeutic Value of the Fully-Freezable BRECS-d in the Treatment of Sepsis and SIRS in a Porcine Septic Shock Model

A series of studies will be carried out to test the efficacy of a BRECS-d unit (post-cryopreservation) seeded with primary porcine renal epithelial cells (PPRECs) in the treatment of sepsis using a large-animal model of septic shock. A variety of hemodynamic parameters, systemic cytokines, BAL parameters, systemic myeloperoxidase (MPO), survival time, urine and ultrafiltrate NGAL levels (as an indicator of acute kidney injury (AKI)), systemic free-elastase levels, and leukocyte activation parameters will be assessed and compared in sham- and cell-treated groups.

Rationale

The following parameters will be measured in the large animal studies.
1. Cardiovascular parameters: heart rate; systolic, diastolic, and MAP; cardiac output; systemic vascular resistance; stroke volume; renal artery blood flow; central venous pressure; pulmonary capillary wedge pressure.
2. Pulmonary parameters: pulmonary artery systolic and diastolic pressures, pulmonary vascular resistance, arterial to alveolar O₂ gradient.
3. Arterial blood gases: pO₂, pCO₂, pH, total CO₂.
4. Complete blood counts: hematocrit (indirect measurement of capillary leak), CBC and differential will be measured q 1 hour.
5. Inflammatory indices: systemic serum levels of cytokines (IL-1β, IL-6, IL-8, IL-10, INF-γ, TNF-α), myeloperoxidase, and elastase will be measured q 2 hours.
6. Pulmonary inflammation by BAL fluid parameters will be measured at baseline and 6 hours post-bacterial infusion: protein content (vascular leak); total cell counts with differential; IL-1β, IL-6, IL-8, IL-10, INF-γ, TNF-α, neutrophil myeloperoxidase, elastase. Alveolar macrophages will be isolated from BAL fluid, and various baseline and stimulated levels of cytokines assessed after LPS challenge using methods established in the laboratory.
7. Leukocyte Activation: Blood will be collected in heparinized tubes at baseline and q 1 hour post-bacteria infusion at the pre-BRECS-d hemofilter and post-BRECS-d hemofilter blood ports for the processing of leukocytes for assessment of leukocyte activation
8. Acute Kidney Injury: Urine and ultrafiltrate will be collected q 1 hr for relative comparison of NGAL levels, serum creatinine and BUN will measured at baseline and at the final time point.

Research Plan

Two groups of animals will be evaluated and compared: Group 1 receiving BRECS-d therapy and Group 2 receiving a sham BRECS-d unit containing no cells. Each group will have 11 animals. Outcome measurements will include the above parameters in addition to survival time. Outcome will be considered positive if the BRECS-d units are shown to be effective in treating sepsis as indicated by an overall decreased inflammatory state (as indicated by systemic, BAL macrophage and circulating monocyte cytokine panels and MPO activity), increased cardiovascular and pulmonary function, increased survival time, and a decrease in leukocyte activation. An additional outcome measurement is to compare these above detailed parameters in BRECS-d units that have gone through the cryopreservation process to BRECS-d units that have not been cryopreserved.

Methods

Animal Study Design

The planned experiments this proposal are based on a well-established porcine model of bacteremic septic shock, which was further refined and in which death occurs in 8 hours or less. In brief, pigs weighing 30-35 kg will be used in these studies. All pigs will be treated for 7 days prior to therapy with the antibiotic Baytril in an attempt to eliminate any pre-existing infections that could skew experimental results. Baseline blood cultures will be drawn to assess the animal infection state prior to initiation of the study. Animals with positive baseline cultures that could affect study outcome will be eliminated from the study group set. After administration of anesthesia and intubation, pigs will undergo placement of dialysis catheter, arterial catheter, and Swan-Ganz thermodilution catheter (which are connected to transducers) to monitor arterial blood pressure, cardiac output, and central venous pressures. A Foley catheter is placed in the urinary bladder to measure hourly urine output. Doppler flow probes are placed around a renal artery and connected to a flow meter (available from Transonics Systems Inc.®, Ithaca, N.Y.). After all access lines are in place and secured and baseline parameters and laboratory studies obtained, 200 cc of broth containing 30×10¹⁰ bacteria/kg body weight of E. Coli (serotype 06:K2:H1) is instilled into the animals' peritoneal cavities. To better replicate the human clinical situation, cefriaxione (100 mg/kg) broad spectrum antibiotic will be administered 15 min after bacteria instillation. At this time, 80 mL/kg of crystalloid and 20 mL/kg of colloid (Hetastarch) is administered. Systemic heparinization is administered to maintain patency of the extracorporeal circuit. At the time of bacteria administration, conventional CVVH is initiated with a Fresenius F-40 hollow-fiber dialysis cartridge (HF-1; placed pre-BRECS-d) along with a BRECS-d unit and second F-40 cartridge (HF-2; placed post-BRECS-d), as depicted in both FIGS. 18A and B (i.e., both circuits will be tested). Extracorporeal blood flow is regulated at 200 mL/min to the lumen of HF-1. UF production from the ECS outlet of HF-1 is warmed to 37° C. via a thermal regulator and maintained at a desirable rate to sustain the nutritional and oxygen needs of the cells in the BRECS-d unit, where it passes through the cell chamber and then enters the lumen of HF-2. The lumen outlet UF flow rate of HF-2 is regulated at a rate of 5 mL/min to maintain physiologic pressures, and the 20 mL/min balance of the HF-2 UF exits via ultrafiltration out the ECS UF outlet, where it is once again combined with the blood that comes from the HF-1 lumen outlet and will then be returned to the animal. A balanced electrolyte replacement solution containing bicarbonate base is infused into the blood line pre-HF-1 on a 1:1 volume replacement basis of net UF that will exit the circuit as waste via HF-2 lumen outlet. Continuous volume resuscitation with normal saline will also be employed so that replacement fluid will be increased after initiation of CVVH plus BRECS-d to provide a net balance of 150 mL/h to maintain MAP and cardiac output. All treatment groups will receive identical volume resuscitation protocols. No animals will receive vasopressor or inotropic agents. Animals will be observed until no arterial waveforms can be detected or at 12 hours post-bacterial infusion, at which time the experiment is terminated.

Cardiovascular parameter measurements and samples for biochemical analysis are obtained at various time intervals during the experiment. At baseline and 6 hours post-bacteria infusion, a BAL will be performed for assessment of pulmonary microvascular damage and inflammation in response to SIRS. Leukocytes will be isolated at baseline and hourly post-bacteria infusion for assessment of the effect of cell therapy compared to sham controls on leukocyte activation. Urine and ultrafiltrate samples will be collected hourly for assessment of NGAL levels as an early indicator of acute kidney injury, and serum elastase will be measured to assess neutrophil degranulation.

Test Parameter Methods

Serum chemistries will be measured with an automated chemical analyzer. Cytokine levels will be measured with commercial ELISA assay kits reactive to porcine cytokines: IL-1β, IL-6, IL-8, IL-10, TNF-α, and IFN-γ (R&D Systems). BAL fluid will be obtained for cell counts and cell-type distribution, protein as a measure of vascular leak, myeloperoxidase (MPO) as an indicator of the activated state of neutrophils that have infiltrated the lung air space from the lung tissue, elastase as indicator of neutrophil degranulation and cytokine levels, including IL-1β, IL-6, IL-8, IL-10, TNF-α, and IFN-γ. BAL fluid is obtained with cannulation of the right middle lobe bronchus and infusion of 60 mL of saline utilizing three aliquots of 20 mL each. Each aliquot sample is obtained by brisk injection and slow withdrawal of fluid. MPO will be measured via a standard published method. Protein will be measured via a standard Bio-Rad protein assay kit II. A protocol for measuring the level of leukocyte surface expression by staining freshly isolated whole blood with a FITC-conjugated anti-porcine CD11b antibody has been developed. To assess leukocyte activation, leukocytes will be processed from heparinized blood which is either incubated immediately with a FITC-conjugated anti-porcine CD11b antibody or after a 30 minute exposure to PMA. PMA acts as a positive control/maximal stimulation condition. The antibody/blood mixture is then be fixed with a Becton-Dickenson's “Facs lyse” solution and the leukocyte population will then be analyzed for activated neutrophils, monocytes and eosinophils. The Becton-Dickenson's “Facs lyse” solution serves a three-fold purpose: it fixes the antibody-stained leukocytes for delayed flow cytometry analysis, it destroys the red blood cells, eliminating them from the analysis, and it separates the two granulocyte populations (neutrophils and eosinophils) from each other on the flow cytometer's forward and side scatter plots. Cells are then collected by centrifugation and resuspended for flow cytometry analysis for assessment of leukocyte activation of various cell populations. Early acute kidney injury (AKI) will be assessed by analyzing levels of neutrophil gelatinase-associated lipocalin (NGAL) in both the urine and the ultrafiltrate samples. Recent clinical data have clearly demonstrated that NGAL is an early biomarker for acute kidney injury. The amount of NGAL in the urine and serum is a highly specific and sensitive predictive marker of acute kidney injury. Relative NGAL levels will be determined by western blot using an antibody that cross reacts with porcine NGAL as indicated by the expected 25 kb band on western blot. Differences in NGAL levels should reflect the degree of AM in this animal model under sham and cell treated conditions. Serum elastase will be assessed as an indicator of peripheral blood neutrophil degranulation. Neutrophil elastase is released into the serum as neutrophils undergo degranulation. Under normal circumstances the serum elastase is inhibited by α1-antitrypsin, forming an elastase-α1-antitrypsin dimer. However, if the serum concentration of elastase exceeds the available inhibitor or α1-antitrypsin secretion by the liver is decreased due to hepatic injury, it may be possible to measure the activity of free elastase from the serum. Briefly, freshly isolated serum will be incubated in 100 μl reactions diluted in 1× Hank's buffered saline containing 50 μmol/L MeOSuc-AAPV-AMC (Biomol® International, Inc., Plymouth Meeting, Pa.) a fluorogenic substrate, incubated at 37° C. for 30 minutes. The conversion of fluorogenic substrate to detectable fluorescence will be measured on a microplate reader using the following settings: excitation wavelength, 380 nm; emission wavelength, 460 nm Controls will include samples with no added serum (negative control) and the addition of purified human neutrophil elastase (Biomol® International, Inc.).

Cardiovascular and biochemical data will be analyzed by repeated-measures analysis of variance (ANOVA). Plasma levels of various moieties, lung tissue and BAL MPO and elastase levels, leukocyte activation, NGAL fold over baseline, serum elastase and survival times will be compared utilizing Student's T-test, paired or non-paired as appropriate.

Contemplated Results

As suggested by the preliminary data detailed below, BRECS-d treated animals will have better cardiovascular indices, less pulmonary dysfunction and lung inflammation, and significantly extended survival times as compared to the sham group. BRECS-d treatment will act to inhibit leukocyte activation when compared to sham group.

### Testing of the Freezable BRECS-d Device of FIGS. 15 and 16

As previously mentioned, a fully-freezable device has been designed and tested. This device is illustrated in FIGS. 15 and 16 and, as the development described above indicates, is designed to allow the entire BRECS-d unit to be frozen or cryopreserved while maintaining cell viability and holding subcomponents and also to allow holding the subject's ultrafiltrate. Cryopreservation of the device should be viable in a liquid nitrogen vapor phase at a temperature range of negative 90 to negative 150 degrees Celsius; alternatively, the device could be stored in the liquid phase at negative 190 degrees Celsius. Further, to hold the subject's ultrafiltrate, the device should be biocompatible, maintain a seal, and withstand sterilization (e.g., use an autoclave at 115 degrees Celsius for 45 minutes or immersion of the device in ethylene oxide at 50 degrees Celsius for 2 to 3 hours) and pressure (e.g., no leaks up to 600 mg Hg). Also, there should be no cracks or leaks in the device.

Prior to clinical application of the entire device, each component of the device is sterilized using, for example, ethylene oxide, gamma radiation, and/or autoclave. Thereafter, the device is frozen for four hours at negative 20 degrees Celsius, followed by an additional four hours at negative 80 degrees Celsius, followed by a transfer to liquid nitrogen vapor or liquid phase, as desired. For clinical application, the device can be thawed in a water bath, the DMSO may be flushed out, the device can be filled with media, and then the device can be connected to an extracorporeal circuit.

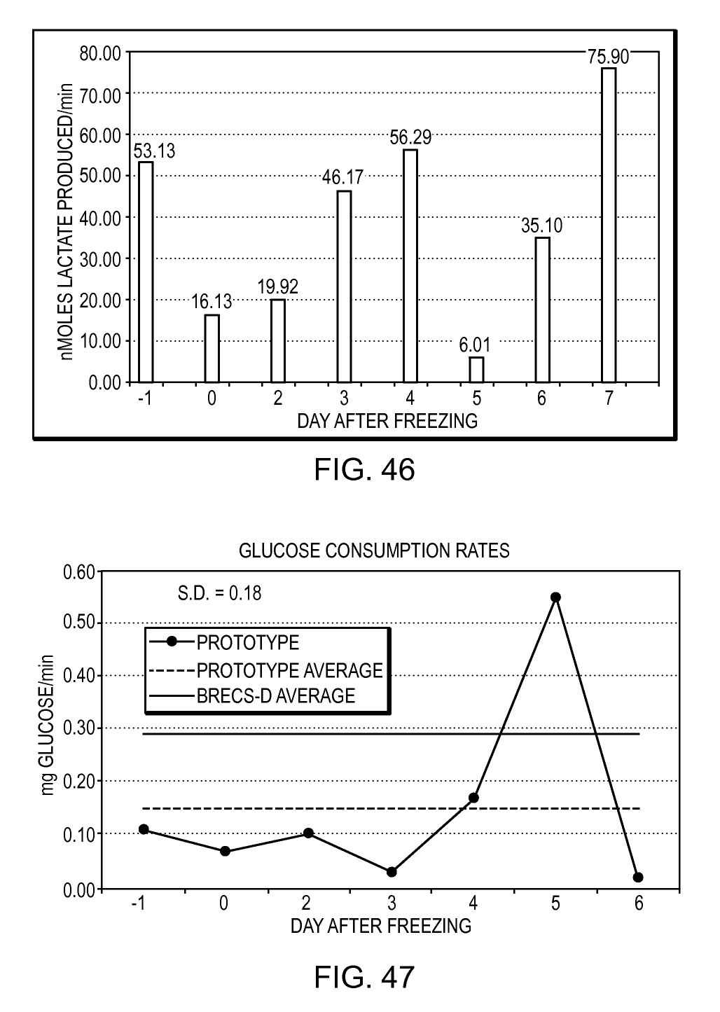

Flow and perfusion studies showed that the freezable BRECS-d of FIGS. 15 and 16 achieved the goals discussed above. First, the device maintained uniform distributed flow throughout, which showed that the device held the subcomponents. The device was shown to be sealed because there was no contamination. The device was also sterilizable because there was no deformation of the device. Under freezing, the device did not crack or show any leaks. The device also withstood pressure, because there were no leaks at 600 mm Hg. Perfusion studies showed that that there was no contamination of the device after four days of incubation at 37 degrees Celsius of cultured samples of non-antibiotic media. Also, the device maintained cell viability as shown in FIGS. 46 and 47. FIG. 46 shows the lactate production by live cells for pre-incubated cells and post-incubated cells after freezing the device. FIG. 47 shows the comparable glucose consumption rates of a BRECS-d device (such as, for example, that shown in FIG. 13; “BRECS-d Average”) and a freezable BRECS-d device (as shown in FIGS. 15 and 16; “Prototype” and “Prototype Average”).

Next, the freezable BRECS-d device of FIGS. 15 and 16 will be further optimized to improve cell viability as measured by oxygen consumption. To this end, more trials of validation tests and a longer cell viability test will be used to determine the statistical significance of this device. Additionally, the seal will be further optimized, for example, by using adhesive and gaskets with the ports (including the inlet and outlet) of the device. A double gasket may also be used in place of the single gasket. Also, other possible materials can be used, such as polysteel or ethylene propylene diene monomer for the gasket and ultra high molecular weight polyethylene for the housing. Thus, not only has a fully-freezable BRECS-d device been developed (FIGS. 15 and 16), but also experimental validation (for example, for use in humans) of the device is contemplated as described above. The cryopreservation and reconstitution of a PPREC-seeded BRECS-d unit is described in Example 8.

### Example 4

### Testing of BRECS-d Peritoneal Fluid Circuits

This example describes the development of extracorporeal circuits (such as, for example, the ones shown in FIGS. 19-21) using peritoneal fluid, rather than blood, using a BRECS-d device (such as, for example, the ones shown in FIGS. 13-16). More particularly, the research described herein is focused on developing large animal models of renal insufficiency for testing the bioartificial nephronal and ultrafiltration prototypes developed previously to assess these renal based devices in an ex vivo system as to their ability to maintain renal function as well as sustain viability over time. This will validate use of the devices in humans. A chronic uremic sheep model was developed for treatment with sorbent regenerated and recycling peritoneal dialysis combined with BRECS units. This model was used to test the BRECS-d device of FIG. 13 in the circuits of FIGS. 19 and 20. Viability of cells in BRECS units will also be evaluated.

### Surgical Protocol and Post-Operation Management

In a pilot study, two different types of catheter were implanted to a sheep peritoneal dialysis model. Compared with an “Ash” Catheter, a double pigtail catheter is less likely to be clotted by omentum and fibrin. Tunneling the catheter as much as possible and/or wrapping external PD catheters with thicker tubing are helpful to better protect catheter from breaking or kinking.

Severe omental adhesion in autopsy was found to compart peritoneal cavity, probably due to reaction to omentectomy during catheter implantation. It was found that one-direction outflow obstruction is very common as a result of fibrin formation within the catheter. Therefore, high dose heparin at 2000 units in 10 mL saline is routinely added to each PD catheter once per day until commencement of PD. To reduce local inflammatory and traumatic response, omentectomy should not be performed when implanting PD catheter.

Optimization of Peritoneal Fluid Recycling with BRECS Unit

Catheters should be used at least 7 days post-op for better healing. In a pilot study, at least 2 liters fluid is needed to maintain appropriate inlet and outlet pressure, which is desirable to ensure PD recycling flow rate at about 100 mL/min A continuous supplement of fresh PD solution to the circuit is desirable in order to compensate for peritoneal absorption. This infusion rate should be at least 100 mL/hour. The first sheep was tested using a BRECS-d unit seeded with lamb kidney cells. Two peritoneal catheters were implanted into the peritoneal cavity, and antibiotic was routinely used to prevent infection. After 1 week post-op recovery, two liters peritoneal dialysate fluid with 1.5% dextrose (Deflex®, available from Fresenius Medical Care North America, Waltham, Mass.) were installed into the peritoneal cavity, with a continuous infusion rate of 100 to 200 mL/hour to supplement intra-peritoneal fluid absorption. The recycling PD circuit consists of two high flux polysulphone filters, whose membrane is both water- and solute-permeable, allowing for differentiated vectorial transport and metabolic and endocrine activity. Any bacterial contaminants with molecular weight larger than cut-off point of polysulphone membrane are held back and/or absorbed by the membrane, thus preventing development of peritonitis. A branch circuit at a desirable flow rate to sustain the nutritional and oxygen needs of the cells was initiated to maintain BRECS viability Immunoprotection of cultured cells in the BRECS unit is achieved concurrent with long-term functional performance as long as the filter membrane is intact. The BRECS is kept in a 37° C. temperature-controlled environment to ensure optimal functionality of the cells. The first sheep was maintained on recycling peritoneal dialysis for 10 days, validating feasibility of the recycling design.

Various compositions of PD solution have been used with this animal model. For example, peritoneal dialysate fluid with 1.5% dextrose solution (Deflex®; code 054-20201, Na 132 mEq/L, Ca 3.5 mEq/L, Mg 0.5 mEq/L, Cl 95 mEq/L, Lactate 40 mEq/L, Dextrose 1.5%, available from Fresenius Medical Care North America, Waltham, Mass.), 4.25% dextrose solution (Deflex®; code 054-20204, Na 132 mEq/L, Ca 3.5 mEq/L, Mg 0.5 mEq/L, Cl 95 mEq/L, Lactate 40 mEq/L, Dextrose 4.25%, available from Fresenius Medical Care North America, Waltham, Mass.), or Extraneal™ solution (Na 132 mEq/L, Ca 3.5 mEq/L, Mg 0.5 mEq/L, C; 96 mEq/L, Lactate 40 mEq/L, Dextrose 0%, Icodextrin 7.5%, available from Baxter International, Inc., Deerfield, Ill.) are used. For human subjects, peritoneal dialysate fluid with 2.5% dextrose solution (Deflex®; code 054-20202, Na 132 mEq/L, Ca 3.5 mEq/L, Mg 0.5 mEq/L, Cl 95 mEq/L, Lactate 40 mEq/L, Dextrose 2.5%, available from Fresenius Medical Care North America, Waltham, Mass.) can be used.

### Biochemical Parameters of Sheep in Recycling PD Circuit

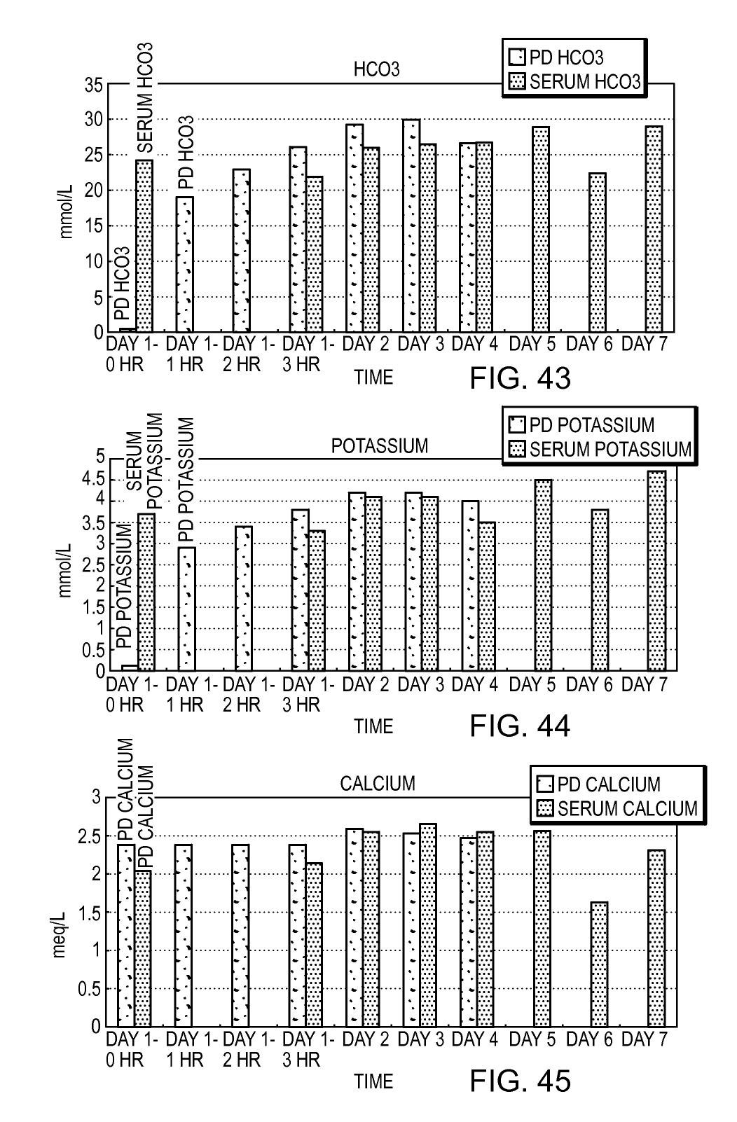

Although no potassium PD solution is used in this study, there are no significant changes in serum sodium and potassium concentration, probably compensated by the food intake of the sheep. There is a slight increase of calcium from 2.04 mEq/L at baseline to 2.31 mEq/L at day 7, but is still in the physiological range. Arterial blood gas was stable during the whole study period, reflecting a good ventilation and acid-base balance. The sheep's glucose metabolism is well-maintained even continuously exposed to high glucose environment, reflected by stable blood glucose level (69 mg/dl at day 1, 70 mg/dl at day 2). This data is displayed in FIGS. 42-45. The results shown in FIGS. 42-45 combine data from tests involving both the extracorporeal circuit of FIG. 19 and the extracorporeal circuit of FIG. 20.

### Prevention and Treatment of Infection

Prophylactic antibiotic was given intravenously. Data showed that combining nafcilin with gentamicin at an appropriate dose to balance the benefit of peritonitis prevention and the risk of pseudocolitis is beneficial. The first sheep developed peritonitis at day 4, presenting cloudy and bloody effluent. PD solution culture showed pseudomonas growth. The second sheep also caught a sepsis because of a pre-existing pneumonia and peritonitis. Thus, prevention and treatment of peritonitis in a prolonged treatment is desirable. Therefore, modifications (FIG. 20) were made to the protocols, which include redesigning the PD recycling circuit to reduce branch number, pre-connecting all tubing in the system and sterilizing it before use, prolonged antibiotic application, and asepsis manipulation. Further modifications included increasing the surface area of the dialysis filter and filtering PD from the subject pre-BRECS and post-BRECS before returning to the subject. Also set up were a standard diagnosis and treatment protocol for sheep peritonitis, which include clinical diagnosis, laboratory diagnosis, empiric antibiotics selection, and specific antibiotics treatment.

### Further Results of Ex Vivo Testing of the Circuits Shown in FIGS. 19 and 20

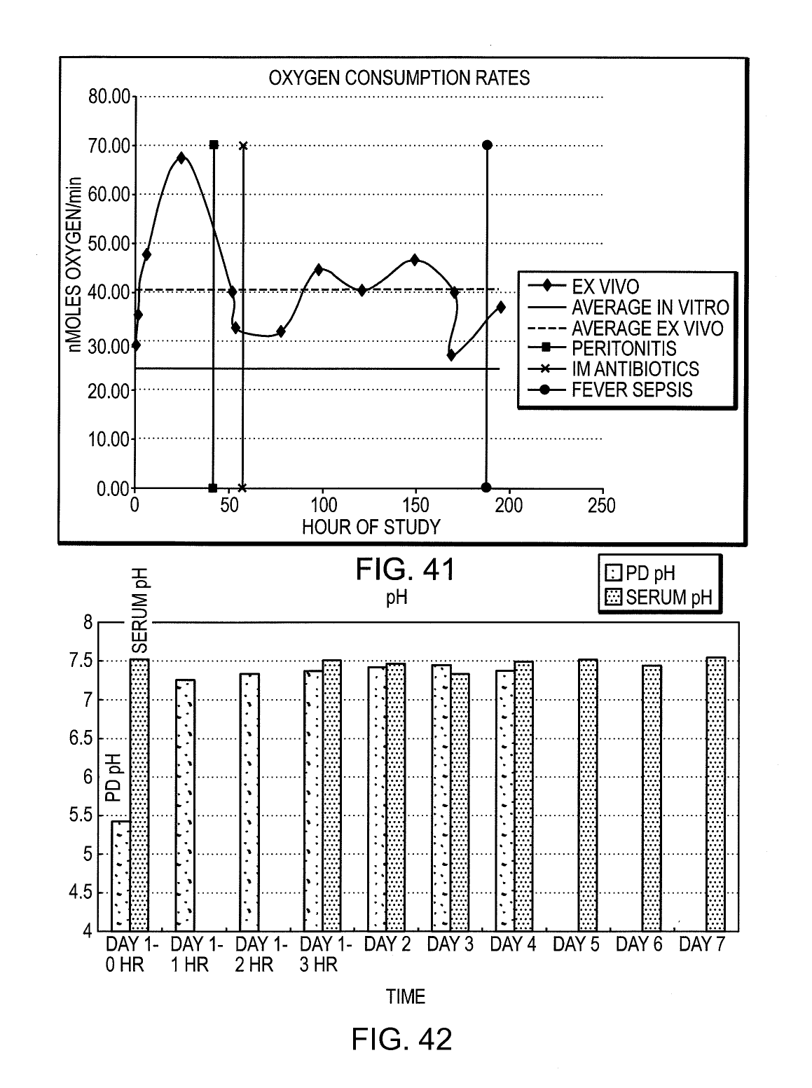

Further results of the ex vivo testing of the circuits shown in FIGS. 19 and 20, using the BRECS-d device of FIG. 13, are depicted in FIGS. 40 and 41. The results shown in FIGS. 40 and 41 combine data from tests involving both the circuit of FIG. 19 and the circuit of FIG. 20. FIG. 40 shows the LREC oxygen consumption rates of ex vivo testing of the circuits illustrated in FIGS. 19 and 20 compared to average in vitro testing of the device illustrated in FIG. 13. FIG. 41 shows the same data of FIG. 40 in comparison with other known data for peritonitis, IM antibiotics, and fever sepsis. These results indicate the ability of the peritoneal dialysis recirculation circuit to sustain the cells through all the conditions that the subject was experiencing. These data also show the effects that the systemic conditions had on the metabolic properties of the cells.

### Conclusions

This research has set up systemic approaches of peritoneal dialysis catheter implantation, post-operation management, and a scheme of peritoneal fluid recycling circuit. The validity and feasibility of current designs will be further tested in a chronic uremic animal model. The peritoneal dialysis catheter implantation and post-operation management minimize the possibility of peritonitis and fibrin formation. Also, the peritoneal fluid recycling circuit is applicable to chronic uremic sheep models as well as other mammals, including humans, with BRECS unit viability maintained over time.

### Sorbent System

Several sham operations were performed with a sorbent delivery system from Renal Solutions, Inc. A sorbent regeneration system may be put into the circuit in parallel to the BRECS circuit. The used peritoneal dialysate will be regenerated at an interval of 4 times per day, with each regeneration lasting for about 1 hour, which is very close to inflow and drainage duration for one PD exchange in clinical practice. To ensure solute and water clearance, the speed of the infusion pump in the sorbent system will be programmed to ensure the final concentration of vital solute exactly the same as fresh dialysate.

Freezable BRECS-d Device with a Peritoneal Fluid Circuit

The freezable BRECS-d device, of FIGS. 15 and 16, also can be used in a peritoneal fluid circuit, such as, for example, the circuit shown in FIG. 21. This circuit can be tested in a similar manner as the circuits of FIGS. 19 and 20. It is contemplated that using the freezable BRECS-d device will obtain similar results. Example 7 describes an uremic animal model utilizing the BRECS-d device of FIGS. 15 and 16 and using a continuous flow peritoneal dialysis-based extracorporeal circuit as shown in FIG. 55.

### Example 5

### Testing of a Miniaturized BRECS Device Based on Hollow Fiber and Microcarrier Technology

This example describes the further design and testing of the device illustrated in part in FIGS. 17A and B (referred to in this Example as the “BRECS-h”). The research described herein discusses a miniaturized bioartificial renal cell system (BRECS) device based on hollow fiber and microcarrier technology. The device can be manufactured under conditions that allow for cells grown on microcarriers to be cryopreserved and stored at clinical sites for easy deployment, allowing this technology to be used in a number of indications and settings. As discussed above in Example 2, results of the work done with the larger RAD system served as initial proof-of-principle for these miniature BRECS-h devices.

A BRECS-h device will be fabricated that allows for cryopreservation/thawing under commercial manufacturing/clinical use—based conditions. The effects of cryopreservation on the BRECS-h viability and functionality will be determined under in vitro conditions. Additionally, conditions will be developed to optimize cell number in the BRECS-h unit, and BRECS-h maintenance parameters will be assessed to gain insight as to optimal conditions and for sustaining cell viability and functionality under simulated therapeutic circuit conditions. This research will further optimize the BRECS-h device in a pre-clinical large animal model of SIRS and sepsis, which can then be used to validate the device, for example, for use in humans.

The BRECS-h design allows for a simplified therapy circuit that uses a peripheral catheter access and a single blood pump system, such as the circuit shown in FIG. 22, allowing for treatment to occur in a clinical setting, such as, for example, in a hospital setting outside the ICU.

### BRECS-h Clinical Use

The BRECS-h design utilizes cells grown on microcarriers or particles which are then resuspended in either a biomatrix 3-D gel or physiologic buffer, followed by seeding into the lumens of a hollow fiber bioreactor. The following method of deployment to the research/clinical site is contemplated. First, a tube of cryopreserved cells on microcarriers will either be mixed with a tube of pre-measured biomatrix or physiologic buffer. The cryopreserved cells and infusate medium would be stored at the research/clinical site or a central storage facility until needed. When needed, cryopreserved cells would be thawed, mixed with the biomatrix material or buffer, and syringe-loaded into the treatment device. Alternatively, the cryopreserved cells on microcarriers, already mixed with the biomatrix material or buffer (with both frozen), can be thawed and syringe-loaded into the treatment device. The cryopreserved tube could contain varying numbers of cells for dose-dependent therapy.

### BRECS-h In Vitro Data

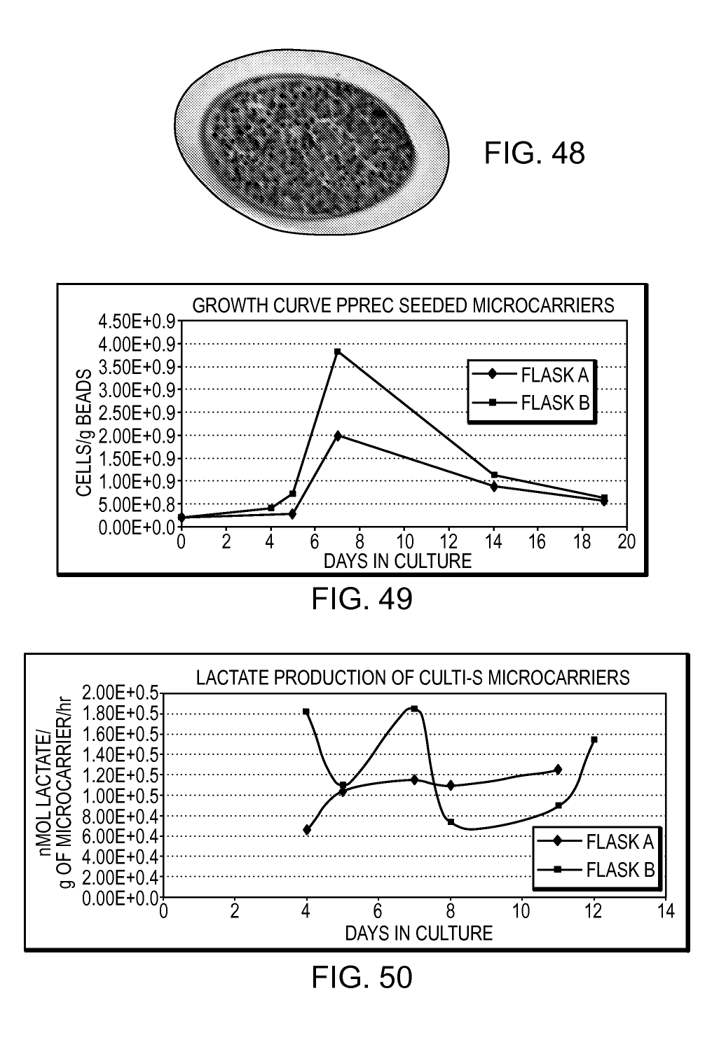

Hollow fibers have been successfully seeded with cells at near tissue density. FIG. 48 shows a hollow fiber, similar to the fibers to be used in the BRECS-h unit, seeded with HEP-G2 cells, an immortalized hepatocyte cell line, and grown in culture for 4 weeks. The cells appear viable, even in the central core of the fiber.

The initial BRECS-h studies were set up to determine optimal methods to obtain a hollow fiber loaded cartridge with 10⁸ cells per BRECS-h unit. Because PPRECs are polarized epithelial cells that typically align to allow for apical and basolateral homogenous configuration, it was decided to first try seeding the cells in a 3-D matrix suspension, rather than cells alone, as with the HEP-G2 cells. Upon histologic assessment of cells resuspended in a 3-D matrix followed by seeding into a hollow fiber cartridge, it appeared desirable that the PPRECs use an attachment surface for optimal cell viability and expansion (data not shown). Study design was then shifted from free cells resuspended in a 3-D matrix, to cells grown on microcarriers, which are then either added to a 3-D matrix or physiologic buffer and seeded into hollow fibers.

### Initial Microcarrier Studies

Studies were undertaken to determine the feasibility of using a hollow fiber/microcarrier system to achieve a BRECS-h unit containing 1×10⁸ cells. Cultispher® S microcarriers were used (available from HyClone, Inc. South Logan, Utah). They are a high thermally-stable, heavily cross-linked, porous gelatin bead. FIG. 49 shows growth curves of two flasks (A, B), each containing PPREC-seeded microcarriers, over a 21-day time course. As can be seen in the graph, cell densities of 2×10⁹ cells per gram of microcarrier were achieved. Also, there is a peak of cell growth at day 7 followed by a rapid decline in cell number per gram of microcarrier, perhaps indicative of nutrient and oxygen limitations in the spinner flask system at the very high cell densities of 2×10⁹ cells/gm of microcarrier or greater. The above data indicated that 0.25 g of microcarriers can readily support 1×10⁸ cells. The volume of cell/microcarrier suspension containing 1×10⁸ cells/0.25 g is approximately 4 mL and, when combined with 3.5 mL of alginate (one of the 3-D matrices of interest) or physiologic buffer, can be loaded into the lumen of a BRECS-h unit. In the case of the alginate condition, calcium chloride can be added to the BRECS-h ECS for gelation. A BRECS-h unit with luminal fill volume of 7.5 mL would be approximately 14 cm in length and 2 cm in diameter, and would contain approximately 2000 hollow fibers of 360 μm inner diameter and 400 μm outer diameter. These dimensions accomplish the desired radial distance of 100-200 μm for oxygen and nutrient delivery to cells at a central core under tissue density conditions. This miniaturized device could be easily deployed in a variety of clinical settings utilizing a single blood pump. For example, FIG. 22 shows a therapy circuit utilizing the BRECS-h treatment device of FIGS. 17A and B.

### Initial Microcarrier Seeded BRECS-h Units

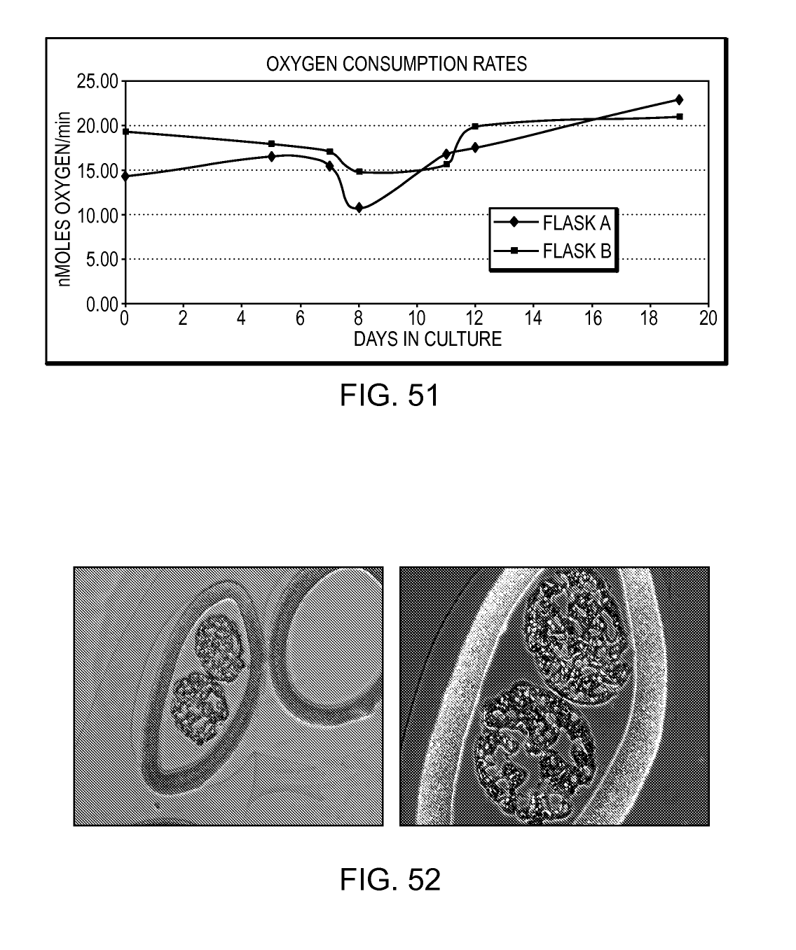

PPRECs were seeded on gelatin-based Cultispher® microcarrier beads (available from HyClone, Inc.). These PPREC-seeded microcarriers were then grown in spinner flask culture for 21 days with samples taken for lactate production, oxygen consumption, and cell counts every day for two weeks and every other day for the last week. FIG. 50 shows lactate production per gram of microcarrier per hour in culture over the first 12 days in culture, with FIG. 51 displaying oxygen consumption over the first 19 days in culture. The decrease in lactate production and oxygen consumption at day 8 in culture may be indicative of nutrient and oxygen limitations to support the exponential growth seen between days 6 and 8 in FIG. 49.

At 5 days in culture, 150 μL of concentrated microcarriers were seeded into mini-hollow fiber devices containing 3 single hollow fibers per device (hollow fiber dimensions, 500 μm inner diameter, 660 μm outer diameter). Units were seeded under two conditions: suspended in a three dimensional alginate matrix and in renal cell culture media. The microcarrier seeded hollow fibers were maintained in culture for 5 days and then fixed for histologic examination. FIG. 52 shows a sample stained section. As can be seen, the microcarriers are contained in the hollow fibers at a loose packing density.

### Initial Microcarrier Cryopreservation Studies

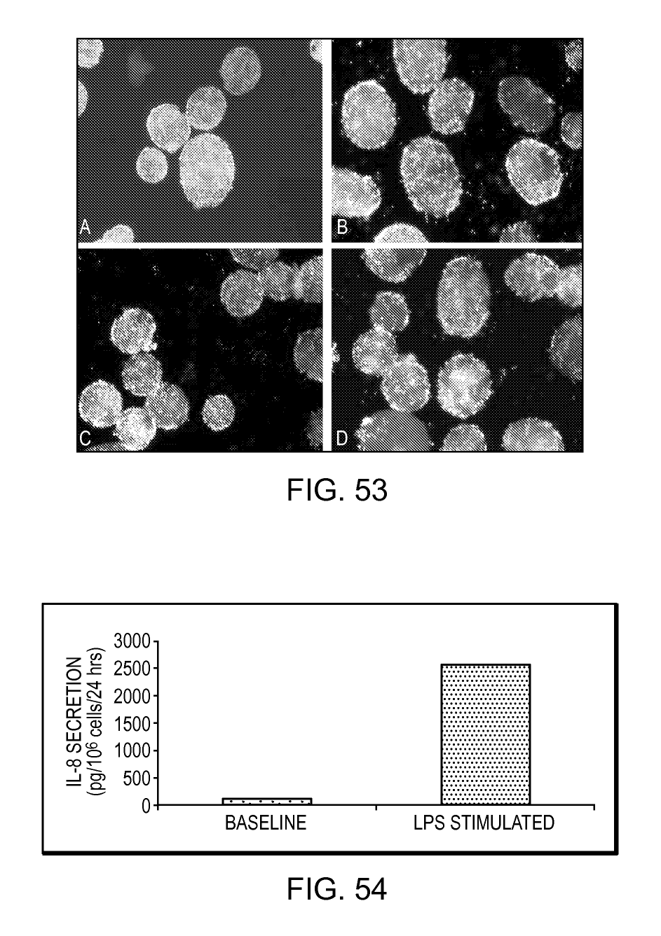

To determine the feasibility of cryopreservation of the Cultispher® microcarriers, the following study was designed. Samples of microcarriers were taken from a spinner culture flask. Samples were taken for cell counts and DAPI staining. Microcarriers were frozen in one of three freezing solutions: CryoStor™ CS5, a cryopreservation medium containing 5% DSMO, (available from BioLife Solutions, Inc., Bothell, Wash.), 5% DMSO in renal cell media, or 10% DMSO in renal cell media. A 100 μL sample of concentrated microcarriers were placed in each of the respective freezing solutions with a final volume of 1 mL. The microcarriers were gently frozen by gradual temperature decrease in a Mr. Frosty freezing container (available from Nalge Nunc International, Rochester, N.Y.) filled with isopropyl alcohol for four hours at −20° C. and then transferred to liquid nitrogen for five days. The microcarriers were then quick thawed in a 37° C. water bath. Media was added drop-wise to a final volume of 10 mL. The microcarriers were incubated in 15 mL conical vials with 10 mL of media for 18 hours on a test tube rocker at 37° C. The microcarriers were then spun down, supernatant was removed, followed by trypsin digestion and trypan blue cell count to assess percent recovery vs. pre-thaw cell number estimates per vial and percent viability. FIG. 53 shows a comparison of the microcarriers before and after cryopreservation in the three cryopreservation solutions. As can be seen in FIG. 53, the microcarriers are visibly more sheared in appearance around the periphery post-cryopreservation than in the fresh, non-cryopreserved sample. Additionally, free cells can be seen in the post-cryopreserved fields.

Table 2 summarizes the results from the three different cryopreservation solutions, with respect to percent recovery post-thaw of a reconstituted vial of cell/microcarriers vs. the estimated pre-thaw cell number per frozen vial of cell/microcarriers, the percent viable vs. dead cells (via trypan blue exclusion) in the post-thaw recovered cells and the percent viable cells in the post-thaw vial compared to the estimated cell number of the cell/microcarriers in the pre-thaw vial. The data show beneficial results from the cryo-protective agent, CryoStor™ CS5. The “% Post-Thaw Recovery of Estimated Pre-Thaw Cell Number” column shows the percent ratio of the number of cells that were frozen as assessed by trypsinizing a sample of the beads aliquoted in freezing solution and staining with trypan blue in comparison to the number counted when trypsinizing a sample of the beads under each condition post-thaw and staining with trypan blue. The number of both viable and non-viable cells are included in the cell count. The “% Death of Post-Thaw Recovery” column shows the percent ratio of the post-thaw dispersed cells that, when counted, are not viable based on trypan blue exclusion assay. The “% Viable Cells of Post-Thaw Recovered” column shows the percent ratio of post-thaw viable cells as a fraction of the total number of cells determined by cell dispersion from the microcarriers followed by cell counts (the data from the “% Post-Thaw Recovery of Estimated Pre-Thaw Cell Number” column), rather than the pre-thaw seeded cell number per gram of microcarrier. The “% Viable Cells of Estimated Pre-Freeze Cell Number” column shows the percent ratio of post-thaw viable cells as a function of the total number of cells prior to freezing, where total cells are the number of cells seeded per gram of microcarrier prior to the freeze/thaw process. The CS5 condition had 100% recovery of cells with almost 80% viability.

TABLE 2
Table showing the results of freeze-thaw effects for various freezing
media. The overall percent recovery is highest for CryoStor ™ CS5,
however cell viability is better for 5% DMSO.

| | % Post-Thaw | | | % Viable |
|---|---|---|---|---|
| | Recovery of | % Death | | Cells |
| | Estimated Pre- | of | % Viable Cells | of Estimated |
| Freezing | Thaw Cell | Post-Thaw | of Post-Thaw | Pre-Freeze |
| Medium | Number | Recovery | Recovered | Cell Number |
| CS5 | 100.00% | 20.80% | 79.20% | 79.20% |
| 5% DMSO | 20.32% | 9.30% | 90.70% | 18.43% |
| 10% DMSO | 28.60% | 18.10% | 81.90% | 23.42% |
| Overall | 49.64% | 16.07% | 83.93% | 40.35% |

### BRECS-h Functional Assessment Criteria

### Effect of PPREC-Conditioned Media on Activated Leukocytes

The assay discussed above in Example 2 regarding detection of leukocyte activation (under “Effect of PPREC-conditioned Media on Activated Leukocytes”) will be used to determine if conditioned media collected from the BRECS-h units after various manipulations and incubated with normal isolated porcine leukocytes under non-LPS and LPS stimulated conditions inhibit activation of leukocytes as compared to the non-manipulated BRECS-h media. Anti-porcine CD11b antibody staining of the leukocytes will be assessed and used as a biomarker index of the BRECS unit's ability to inhibit the activation of leukocytes. It is contemplated that the leukocytes, when exposed to PPREC-conditioned media, will be less activated at each condition than leukocytes not exposed to PPREC-conditioned media, as determined by CD11b flow analyzed studies.

### PPREC IL-8 Secretion

Renal epithelial cells secrete low, but measurable levels of IL-8 under baseline conditions with significant increases post-LPS stimulation. FIG. 54 shows IL-8 secretion rates of PPRECs grown to confluence on culture plates under baseline and 24 hours LPS stimulated conditions. The baseline IL-8 secretion level was approved by the FDA to be used as a release criteria for the renal assist device in the Phase I/II and Phase II clinical trials discussed above in Example 2 (under “Clinical Evaluation of Efficacy of Renal Cell Therapy”), in which the human renal cells used in the RAD display the same characteristics and are isolated using similar protocols as the Porcine cells used in the BRECS-h. This precedent with the FD A, in addition to patients with SIRS and sepsis being the target population for BRECS-h therapy, warrant IL-8 baseline and LPS stimulated secretion rates to be used as a measure of functional assessment when determining the affect of various parameter modifications on the BRECS-h. The data implies that renal epithelial cells normally produce a low level of IL-8 and, when exposed to the endotoxin in LPS, significantly upregulate their secretion of this molecule. Thus, these renal cells may have an impact on the septic/inflamed disease state.

As shown above, the BRECS-h design allows for the device to be both miniaturized and the cells to be cryopreserved, followed by a quick thaw process and syringe loading, thus eliminating the shortcomings of the RAD design.

Development of a BRECS-h Prototype that can be Cryopreserved

The initial data gathered above will be further expanded.

Cell Growth and Seeding

PPRECs will be seeded and expanded on microcarrier beads. Optimal cryopreservation/thaw conditions will be established for the cell-seeded microcarriers. Thawed microcarriers will then either be resuspended in an alginate solution or in a physiologic buffer, seeded into a BRECS-h unit and, if alginate-based, gelled under increased calcium conditions. The BRECS-h system will also be tested in vitro to assess cryopreserved shelf life of the cell-seeded microcarriers 1 week, 1 month, 3 months and 6 months cryostorage.

The following parameters will be measured.
1. Several microcarriers (examples include Cultispher® gelatin-based microcarriers (available from HyClone Inc., South Logan, Utah); HyQ® Spheres™ polystyrene-based microcarriers (available from HyClone, Inc., South Logan, Utah), microcarriers available from SoloHill Engineering, Inc. (Ann Arbor, Mich.); Cytodex® dextran-based microcarrier beads (available from GE Healthcare Bio-Sciences Corp., Piscataway, N.J.), and Sigma-Solohill polystyrene-based microcarrier beads (available from Sigma-Aldrich Inc., St. Louis, Mo.) will be assessed with regard to cryopreservation characteristics. Initial cryopreservation protocols will follow published techniques established for liver cells. In addition several freezing mediums will be tested for optimal recovery, viability and cell function post-cryopreservation. Examples include, but are not limited to, Cryostor™ CS-5, HypoThermosol®-Purge Solution (available from BioLife Solutions, Inc., Bothell, Wash.), 5% DMSO, 10% DMSO, VS55 (a solution containing 24.2% w/v (3.1M) dimethyl sulfoxide, 16.8% w/v (2.2M) 1,2-propanediol, and 14.0% w/v (3.1M) formamide), KYO-1 (5.38-Methylene glycol, 2M DMSO, 0.1M PEG 1000, and 0.00175M PVP K10 in EuroCollins solution), and any additional buffers used for cryopreservation or vitrification of organs for storage. Cell viability assessment criteria will include: cell release from the microcarrier, lactate production, oxygen and glucose consumption, cell recovery, and trypan blue exclusion pre- and post-cryopreservation. Cell functional assessment will include the ability of microcarrier-conditioned media to inhibit leukocyte activation and baseline and LPS stimulated IL-8 production. Both functional criteria will be assessed pre- and post-cryopreservation. It is contemplated that the stimulation potential and IL-8 production will be maintained pre- and post-freeze, as will the other metabolic markers. Because LPS stimulation is an endpoint test, multiple samples of microcarriers from the same spinner flask culture will be assessed for the pre- and post-cryopreservation comparison.
2. Once parameters for cryopreservation and microcarrier cell density are optimized, a cryo-storage shelf life study will be initiated. Cell-seeded microcarriers will be cryopreserved for 1 week, 1 month, 3 months and 6 months, with the established viability and functional criteria assessed pre- and post-cryopreservation. Upon thaw, 4 BRECS-h units will be seeded from microcarriers for each of the 4 cryopreservation time points (4 cryo-time points×4 BRECS-h/time point=16 BRECS-h units).

One consideration is the choice of cryoprotective agent. Cryoprotective agents such as glycerol and DMSO, which increase osmolarity, effectively dehydrate cells during the freezing process and thereby eliminate damaging ice crystal formation, will be considered for optimization. However, due to physical constraints, cells in solid organs, or in this case adhered to solid microcarrier structures, do not adjust as readily as cells in suspension to the abrupt changes in osmolarity inherent to standard cryopreservation protocols. Because microcarrier cultures have a high cell density, they should be transitioned quickly to avoid nutrient depletion; however, the diffusion of cryoprotectants is variable across solid structures and should be controlled to avoid mechanical stress to the surface cells while being allowed to permeate the microcarrier interior. A balance should be reached between these two factors.

Methods will be developed that optimize loading of cryopreserved PPRECs grown on microcarriers into hollow fibers post-thaw. It is contemplated that the cell-seeded microcarriers will be able to withstand cryopreservation with no detrimental effect on PPREC viability and function.

Optimization of Cell Number per BRECS-h Unit

Several methods will be tested to optimize both the number of cells per microcarrier and the loading capacity of microcarriers into the BRECS-h unit. Microcarrier cultures seeded and maintained under various conditions will be evaluated over time to obtain growth curves in culture to assess the number of cells per gram of microcarrier. The determined optimal growth parameters will then be used to test several loading protocols into the BRECS-h unit to develop a method that allows for maximal microcarriers per BRECS-h.

The following parameters will be measured.
1. PPRECs will be seeded and expanded on microcarrier beads. A variety of seeding techniques will be employed to maximize cell number per gram of microcarrier. These include varying the initial cell to gram microcarrier seeding ratio, performing multiple seeding of the same microcarriers, modifying the microcarrier concentration in the spinner flask culture and establishing spinner flask maintenance protocols that decrease nutrient and oxygen limitations in the spinner flask cultures. Assessment criteria for cell viability and functionality will include cell growth curves as determined by cell counts with trypan blue staining, lactate production and oxygen depletion in culture, length of time for cell growth plateau, and length of time the microcarrier can sustain cell growth/viability once the plateau is reached. Data detailed above suggests that spinner flask culture maintenance methods need to be established with attention to potential nutrient and oxygen limitations when spinner flask cultures begin an exponential growth phase.
2. Protocols will also optimize maximal cell-seeded microcarrier packing density in the hollow fibers of the BRECS-h. Several techniques will be employed to confirm which single process or combination of processes yields a tightly packed BRECS-h unit. Cartridge orientation (1-horizontal vs. 2-vertical), in addition to loading direction (3-against or 4-with gravity), 5-pressure loading via clamping of the luminal exit port with careful monitoring of pressure build-up, and 6-multiple seedings alternated between centrifugations will be employed to optimize the microcarrier packing density. Assessment criteria for cell number will include lactate production, oxygen depletion, glucose consumption rates, histologic assessment. (6 techniques×4 BRECS-h units per technique=24 BRECS-h units)

It is expected that these methods will allow for BRECS-h units containing 10⁸ to 10⁹ cells. Further optimization of methods that allow for a slow, steady exchange of media with a flow through system that has a “catch trap,” so microcarriers are not removed with the spent media, may be desirable. Supplemental oxygen may also need to be supplied to the spinner flask system. Due to the potential of the hollow fiber packing density to make a non-homogeneous delivery of oxygen and nutrients to the encapsulated cell-seeded microcarriers, it is desirable to design hollow fibers with smaller outer diameter, which would be potted into the BRECS-h casing.

Determination of BRECS-h Maintenance Conditions that Sustain Optimal Cell Viability and Functionality

Conditions for flow rates, pressure gradients (both across the ECS to lumen path and along the pre- to post-ECS path), luminal buffer or alginate monomer composition, fiber length, and fiber density in the ECS housing will be assessed to determine which parameters support optimal oxygen and nutrient gradients to the cells grown on microcarriers and contained in the hollow fibers of the BRECS-h unit. Conditions will be simulated that will allow for the BRECS-h device to be assessed with respect to viability and functionality under parameters seen in an ex vivo extracorporeal circuit, such as the circuit of FIG. 22, over an extended period of time. The optimal BRECS-h microcarrier packing density, discussed above, will be tested in vitro under various conditions that assess cartridge and circuit parameters.

Various conditions for flow rates, pressure gradients (both across the ECS to lumen path and along the pre- to post-ECS path), luminal buffer or alginate monomer composition, fiber composition, fiber outer diameter and wall thickness, fiber length, and fiber density in the ECS housing will be assessed to determine which parameters optimally support oxygen and nutrient gradients to the cells grown on the microcarriers contained in the hollow fibers of the BRECS-h unit. Conditions will be simulated that will allow for the BRECS-h to be assessed with respect to functionality under parameters seen in an ex vivo extracorporeal circuit with respect to oxygen and nutrient delivery and pressure gradients. In order to more closely match the clinical circuit setting, heparinized bovine blood will be used in the in vitro test circuit for some of the studies in this component of the research plan. Functional assessment criteria will include lactate production, oxygen depletion, glucose consumption rates, histologic assessment, leukocyte conditioned media assay and IL-8 baseline and LPS stimulated secretion rates. Many of these studies (luminal buffer or alginate monomer composition, fiber composition, fiber outer diameter and wall thickness) will initially be performed in mini-hollow fiber units to test optimal parameters. Once these have been determined, then the BRECS-h units will be used for testing final parameters for flow rates, pressure gradients and fiber packing density. 8 BRECS-h units will be tested for each of these parameters with 8 BRECS-h unit being tested with the final optimal combination ((3 test parameters×8 BRECS-h per parameter)+8 BRECS-h for optimal combination=32 BRECS-h units).

It is contemplated that the cells in the BRECS-h will prove both viable and functional with respect to the assessment test parameters. Conditions for flow through the BRECS-h ECS will allow for adequate nutrient and oxygen delivery over the anticipated therapeutic application time period. Hollow fiber length and/or hollow fiber packing density inside the ECS may be varied. A membrane oxygenator may be used in the pre-BRECS-h section of a circuit to enhance further delivering oxygen to the cells. Such an optimized BRECS-h unit may be tested in a pre-clinical large animal model of sepsis and SIRS that would target the septic shock and SIRS patient population. This model would confirm the utility of the BRECS-h and validate its use in humans.

### Example 6

### Collagen Coating and Post-Seeding Culture of Substrates, Such as Carbon Nb Disks

This example describes a protocol for coating substrates according to the invention, such as the Nb-coated carbon disks of FIGS. 13-16, with collagen IV, seeding such substrates with live cells, and post-seeding culture of these seeded substrates.

Collagen Coating of Carbon Nb Disks

The technique for passive coupling of proteins to the Dynamatrix carbon Nb disk matrices recommended by Dynamatrix, the manufacturer, is a one-sided static coating technique. The protocol from the manufacturer calls for disks to be coated by placing full tuning fork assemblies, with four disk matrices, each into tissue culture plates and loading 150 μl of protein solution onto each individual disk. A concentration of 10 μg/mL is recommended by the manufacturer for the protein solution, but the protocol states that other concentrations may be appropriate depending on the specific protein and application. After the solution is added, there is an incubation step of one hour at room temperature to allow for protein adsorption onto the matrices. Excess fluid is then aspirated, and the matrix units are dried at room temperature in a sterile hood for one hour prior to cell seeding.

In order to optimize collagen IV coating of disk matrices, the distribution of collagen IV on the disks achieved by static coating was compared to an alternative coating technique in which collagen solution was loaded onto each side of the disk, and centrifugal force was applied after loading. The applied force was intended to push collagen solution into the center of the matrix in an attempt to produce more uniform collagen coverage throughout the matrix than the coverage achieved by the manufacturer's static loading protocol. The distribution of collagen through the disk matrices was evaluated qualitatively by assessing the signal of fluorescent-conjugated collagen IV on the top, bottom, and cross-sectional middle of disks coated by both single-sided static and double-sided centrifugation techniques.

The manufacturer's static seeding technique resulted in heavy coverage of the top of the carbon Nb disk, moderate coverage of the bottom of the disk, and minimal coverage of the middle of the disk. The bottom of the disk showed a strong collagen signal around the edge which rapidly decreased toward the center of the disk, implying that most collagen solution passed around the disk instead of through it to fill the inner matrix. An amended static technique in which collagen was loaded to both sides of the disk resulted in heavy coverage of the top and bottom, but still resulted in minimal coating in the middle of the disk. Since collagen coating occurs by protein adsorption, the lack of coating in the middle of the disks is probably due to passive loading of the collagen to the top of the disk, allowing protein solution to simply pass around the edges of the disk. Without any force applied to push collagen through the disk, the center of the disk does not have adequate contact with collagen to allow for adsorption.

Double-sided coating with force applied by a one-minute 14.84×g (300 rpm) centrifuge spin cycle on each side resulted in similar collagen coating of the top of the carbon Nb disk and improved coating of the middle and bottom of the disk compared to static coating. The applied centrifugal force improves the distribution of collagen solution throughout the disk and results in more uniform collagen adsorption by ensuring that collagen passes into the center of the disks where it can be adsorbed during incubation. The coating of the bottom surface of the disk is uniform as a result of loading collagen to both sides, in contrast to the non-uniform coating of the bottom surface of the disk loaded on only one side.

From the results of these collagen coating studies, the protocol for coating carbon Nb disks with collagen IV has been optimized to a double-sided centrifugation coating technique. Collagen solution is prepared at a concentration between about 0.75 ng and about 1 μg collagen IV per cm² of disk matrix to be coated. Prior to coating, all disks are removed from tuning fork assemblies and placed into separate wells of a 48-well cell culture plate. After collagen solution is adjusted to a pH between 7.0 and 7.5, 75 μl of collagen solution is loaded to the top of each disk. Immediately following loading, the culture plate is spun at 300 rpm for one minute. The disks are then incubated in a laminar flow hood at room temperature for one hour to allow for collagen adsorption. Each disk is then turned over, and 75 μl of collagen solution is added to the second side. The culture plate is again spun at 14×g (300 rpm) for one minute, followed by a one-hour incubation at room temperature to allow for adsorption. The disks are then transferred to a new culture plate for cell seeding.

Cell Seeding onto Carbon Nb Disks

The technique for cell seeding onto the Dynamatrix carbon Nb disk matrices recommended by the manufacturer is a one-sided static seeding technique similar to the collagen coating protocol described above. The protocol calls for disks to be seeded by placing full tuning fork assemblies with four disk matrices each into tissue culture plates. A 75 μl cell suspension is then added drop-wise to each individual disk. The total number of cells to be seeded onto each unit is recommended within the range 2.5×10⁵ to 5.0×10⁵ cells, but the protocol states that other seeded cell totals may be appropriate depending on user need and experience. After the cell suspension is loaded, an incubation step of 2-4 hours in a humidified incubator allows for adhesion of cells onto the matrices.

To optimize the seeding of renal epithelial cells onto the carbon Nb disks, distribution of cells throughout the matrices achieved by manufacturer-recommended protocol was compared to an amended protocol in which cells were statically seeded to both sides of the disk. A disk seeded following manufacturer protocol was seeded on one side and incubated for 3 hours, while a disk seeded by the amended protocol was seeded on both sides with a 1.5 hour incubation following seeding onto each side. Distribution of cells throughout the disk matrices was evaluated qualitatively by assessing the signal of fluorescent cell stain on the top surface, bottom surface, and cross-sectional middle of disks comparing manufacturer's standard protocol and the amended double-sided protocol.

The amended double-sided seeding technique resulted in an improved, more uniform distribution of cells throughout the carbon Nb disk as compared to the manufacturer's one-sided seeding technique. Cell coverage was especially improved in the middle and on the bottom surface of the two-sided disk.

One-sided seeding demonstrated heavy cell coverage of the top surface of the disk, moderate coverage of the middle of the disk, and minimal coverage of the bottom surface of the disk. The difference in cell coverage of the top and bottom surfaces results in disks that are sided and could affect the performance of the disks in the BRECS-d units. The one-sided seeding disk shows that the renal cells are able to pass through the disk pores and attach to the interior without external applied force, but loading cells from only one side of the disk showed incomplete cell coverage.

Double-sided seeding produced heavy coverage of the top surface, bottom surface, and middle of the disk. Compared to one-sided seeding, cell coverage was much more uniform throughout the double-sided disk. Loading cells on both sides eliminates the previous issues as cell coverage is more uniform between bottom and top surfaces. Double-sided static seeding still allows cells to pass into and attach to the interior of the disk, and the resulting cell uniformity makes this technique a more effective option than single-sided loading.

From the results of the cell seeding study, the optimized protocol for seeding renal epithelial cells onto carbon Nb disks coated with collagen IV is a double-sided static seeding technique. Prior to seeding, carbon Nb disks are coated with collagen IV by the optimized coating protocol discussed above. The coated disks are placed into separate wells of a 48-well cell culture plate for seeding. A cell suspension of renal epithelial cells in a customized UltraMDCK media (available from Lonza Group Ltd., Basel, Switzerland, catalog number 2042042) is prepared with a concentration of 1×10⁶ cells per 75 μl of media. 1 L of UltraMDCK media is mixed with 1 ml of a 500× Insulin, Transferrin, Ethanolamine, and Selenium (ITES) mixture for a final concentration of 0.5× (available from Lonza Group Ltd., Basel, Switzerland, catalog number 17-8392), 3 μl Triiodothyronine (T3) of 2×10⁻⁶M for a final concentration of 6×10⁻¹²M (available from Sigma-Aldrich, Inc., St. Louis, Mo., catalog number T-6397), 500 μl Epidermal Growth Factor (hEGF) of 2×10⁻⁵M for a final concentration of 1×10⁻⁸M (available from R&D Systems®, Minneapolis, Minn., catalog number 236-EG), and 100 μl Retinoic acid (RA) of 1×10⁻³M for a final concentration of 1×10⁻⁷M (available from Sigma-Aldrich, Inc., St. Louis, Mo., catalog number R-2625). Alternatively, the concentrations of the additives to the UltraMDCK mixture can be of various ranges. For example, the final concentration of the ITES mixture can be of a range from about 0.0 to about 0.5×, the final concentration of T3 can be of a range from about 0.0M to about 6×10⁻¹²M, the final concentration of hEGF can be of a range from about 0.0M to about 1×10⁻⁸M, and the final concentration of RA can be of a range from about 0.0M to about 1×10⁻⁷M. UltraMDCK media is used for renal cells. Alternatively, other media are contemplated for other types of cells, such as other eukaryotic cells, such as other mammalian cells. A 75 μl cell suspension is then added to each disk, followed by 1.5 hours of incubation at 37° C. and 5% CO₂ to allow for adhesion of cells to the matrices. Each disk is then turned over, and an additional 75 μl of cell suspension is added to the second side. A second 1.5 hour incubation period at 37° C. and 5% CO₂ is carried out to allow for cell attachment.

Post-Seeding Culture of Carbon Nb Disks

Post-seeding culture of Dynamatrix carbon Nb disks by manufacturer protocol is performed in the Starwheel system. The system consists of a media reservoir which has a Starwheel spinner connected to the cap. The spinner has eight arms into which tuning fork assemblies having cell-seeded disks are placed. The spinner is magnetic and spins at a set rpm when the Starwheel system is placed on a stir plate. Manufacturer protocol calls for seeded tuning fork assemblies to be placed into spinner arms in a sterile environment. The cap is then secured onto the media reservoir. The reservoir is filled with 175 mL of media through a side arm, and the system is placed on CellGro spinner plates. Finally, the two side arm caps are loosened to allow for gas exchange during culture, and the spinner plate is set to the desired rpm. Culture rpm recommended by the manufacturer is 2 rpm, and the protocol warns against exceeding 10 rpm.

To optimize post-seeding culture of carbon Nb disks seeded with renal epithelial cells and to provide for a more robust manufacturing process, culture in the Starwheel system was compared to culture in a BRECS-d unit (such as the one shown in FIG. 13) with the perfusion of media. Perfusion in the BRECS-d unit would bypass the Starwheel systems and would streamline the device preparation process by eliminating an extra step and minimizing the required handling of the disks. Four tuning forks loaded with four disk matrices each were coated by double-sided centrifuge collagen coating and seeded by double-sided static seeding. Then, each of these four prepared tuning fork assemblies was cultured by four different experimental techniques: manufacturer-recommended 2 rpm Starwheel, a faster 4 rpm Starwheel, 25 mL/min BRECS-d perfusion, and a faster 50 mL/min BRECS-d perfusion. The 25 mL/min and 50 mL/min perfusion rates were chosen because they were calculated as the equivalent media delivery of 2 rpm and 4 rpm Starwheel culture, respectively. Each technique was evaluated for maintenance of renal cells on the disks for a period of two weeks in culture by assessing the signal of fluorescent cell stain on the top surface, bottom surface, and cross-sectional middle of disks by visual inspection using a camera-microscope configuration.

Perfusion in the BRECS-d systems resulted in better cell growth and more effective maintenance of viable cells on the carbon Nb disks than the Starwheel culture systems. After two weeks in culture, both the 25 mL/min and 50 mL/min BRECS disks showed improved cell coverage compared to either of the Starwheel system conditions. There was no noticeable difference in cell coverage between the two BRECS-d perfusion systems, but both had significantly higher fluorescent cell signal than both of the Starwheel systems.

From the results of the post-seeding culture study, an optimized protocol has been developed which does not need to use the Starwheel system. Instead of culturing seeded disks in the Starwheel system prior to use in the BRECS-d system, seeded disk matrices in tuning fork assemblies are placed directly into the BRECS unit. Culture in the BRECS-d is performed by the perfusion of UltraMDCK media through the unit at rate of 25 to 50 mL/min Alternatively, a perfusion rate of between about 10 mL/min and about 200 mL/min can be used.

### Example 7

### Cell-Seeded BRECS-d Devices in Large Animal Applications

This example describes the development of a cell therapy system implemented in two large animal applications, an anephric sheep model of uremia and a porcine model of sepsis. The goal was to evaluate the functionality of cell-seeded BRECS-d units in pre-clinical acute and chronic therapy applications. More particularly, the chronic uremic sheep model was used to test the BRECS-d device of FIGS. 15 and 16 in the circuit of FIG. 55. Additionally, BRECS-d therapy was applied to an acute porcine septic pig model. This model was used to test the BRECS-d devices of FIGS. 13-16 in the circuits of FIGS. 18A and B.

### Assessment of a HREC-Seeded BRECS-d Device Utilizing CFPD in a Sheep Model of Uremia

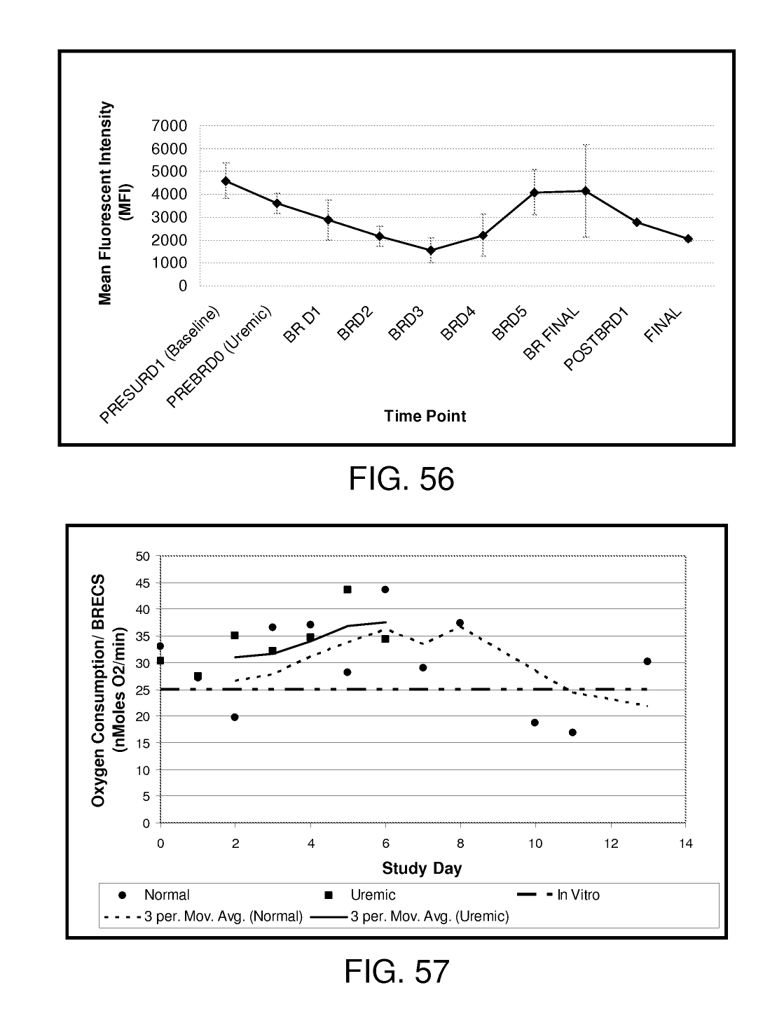

As described in Example 4, a twenty-four hour continuous flow peritoneal dialysis (CFPD) protocol was developed using normal sheep. This protocol utilized the LREC-seeded BRECS-d device of FIG. 13 in circuits as shown in FIGS. 19 and 20. In order to test the functionality of a BRECS-d device in chronic therapy applications, the CFPD protocol was implemented in an anephric sheep model of uremia. Female sheep weighing about 40 kg were used. In brief, two peritoneal catheters were implanted into the peritoneal cavity of each sheep one week before the start of CFPD. Normal animals were treated as described in Example 4. In uremic sheep, sequential nephrectomies were performed at one-week and 1 to 3 days before CFPD. CFPD was initiated in each uremic animal by infusing two liters of commercially available peritoneal dialysate (PD) fluid with 4.25% dextrose into the peritoneal cavity. A continuous infusion of fresh PD solution at a rate of 100-200 mL/hour was maintained to compensate for peritoneal fluid absorption. After at least twelve hours of CFPD in an uremic animal, a BRECS-d device of FIGS. 15 and 16 was incorporated into the circuit of FIG. 55. The tested BRECS-d devices contained up to 10⁸ cells. Experimental duration was limited by animal use committee regulations in the established protocol. Normal sheep were maintained on CFPD for up to 14 days, with BRECS-d therapy for up to 13 days. Uremic sheep were maintained on CFPD for up to 10 days, with BRECS-d treatment for up to 6 of those days (BRD1 through BR FINAL, as shown in FIG. 56). Hemodynamic and chemistry profiles of the animals were obtained and cell viability was monitored during the study period.

Results

CFPD was started and maintained successfully without PD catheter-associated complications or peritonitis in 10 animals (n=3 for normal sheep and n=7 for uremic sheep; average values of the data collected for the CFPD studies were calculated based on the number of animals that remained in the study at a particular time point). Further, CFPD provided a flow rate of ≧80 mL/min in the main circuit and a flow rate of 50 mL/min within each BRECS-d unit without interruption. This was achieved while maintaining a intra-peritoneal dwelling volume of 2 L, a dialysate infusion rate of 400 to 450 mL/hr, and a dialysate drainage rate of 450 to 470 mL/hr. Given this difference between the dialysate infusion and drainage rate, the net change in intraperitoneal fluid volume, otherwise known as the net ultrafiltration volume, in each uremic animal was estimated to be between about 480 mL/day to about 1200 mL/day.

During the study period, viability of cells in BRECS-d units was assessed by measuring O₂ consumption. As shown in FIG. 57, the LRECs in the BRECS-d device of FIG. 13 remained viable, as demonstrated by a mean O₂ consumption rate of 29.7 nMoles O₂/min during the 13 days of cell therapy in normal sheep. The HRECs in the BRECS-d device of FIGS. 15 and 16 remained viable during the 6 days of cell therapy in uremic sheep, as demonstrated by a mean O₂ consumption rate of 35.0 nMoles O₂/min Further, the oxygen consumption rates diverged from historical in vitro performance.

Neither interruption of circuit flow nor cell death occurred during the study period. The volume and hemodynamic state and chemistry profiles were adequately controlled in both models. Uremic control by CFPD measured by small solute uremic parameters was within acceptable ranges in the uremic sheep.

The effect of BRECS-d cellular therapy on the inflammatory state of uremic sheep was assessed (using the device of FIGS. 15 and 16 in the circuit of FIG. 55). The approach measured the level of CD11b, an inflammatory marker on the surface of activated neutrophils, during the course of the uremic study. For the experimental result shown in FIG. 56, blood was isolated from uremic sheep at the following time points: (1) PRESURD1, which was 1 day prior to sheep nephrectomy; (2) PREBRD0, which was the time point just prior to BRECS-d therapy when the sheep were at their most uremic state; (3) BRD1 to BRD5, which correspond to days 1-5 of BRECS-d therapy, respectively; (4) BR FINAL, which was the final day of BRECS-d therapy, in this case day 6, when the BRECS-d unit was removed; (5) POSTBRD1, which was 24 hours following BRECS-d removal; and (6) FINAL, which was the last time point of this study, occurring at 48 hours following BRECS-d removal. As previously described in Example 2, freshly isolated blood was stained with FITC-conjugated anti-sheep CD11b antibody. The antibody/blood mixture was fixed, and the leukocyte population was analyzed by forward and side scatter flow cytometry analysis. The mean fluorescent intensity (MFI) of the FITC-labeled anti-sheep CD11b antibody, which were bound to activated neutrophils, reflected the systemic inflammatory state of the uremic sheep. As shown in FIG. 56, leukocyte activation appeared to be modulated during BRECS-d therapy as indicated by the changing CD11b MFI from the PREBRD0 time point to the BR FINAL time point. Further, there was a reduction in CD11b MFI upon removal of the BRECS-d device starting after the BR FINAL time point. These studies suggest that the HRECs in BRECS-d devices secreted cytokines/molecules which modulated leukocyte activation in uremic sheep.

### Assessment of a PPREC-Seeded BRECS-D Device in the Treatment of Acute Renal Failure Using a Septic Pig Model

The utility of cell-seeded BRECS-d units in an acute therapy application was studied. In this regard, PPREC-seeded BRECS-d units (as shown in FIG. 13-16) in extracorporeal blood circuits (as shown in FIGS. 18A and B) were tested using an acute porcine model of septic shock. The animal study design was previously described in the section titled “Assessment of the Therapeutic Value of the Fully-Freezable BRECS-d in the Treatment of Sepsis and SIRS in a Porcine Septic Shock Model” in Example 3. In brief, 200 cc of broth containing 30×10¹⁰ bacteria/kg body weight of E. Coli (serotype 06:K2:H1) was instilled into the peritoneal cavity of each pig. To better replicate the human clinical situation, cefriaxione (100 mg/kg), a broad spectrum antibiotic, was administered 15 minutes after bacteria instillation. At this time, fluid resuscitation with 80 mL/kg of crystalloid and 20 mL/kg of colloid (Hetastarch) was administered. For this study, a BRECS-d unit (selected from those shown in FIGS. 13-16, each unit embodiment was tested) was incorporated into an extracorporeal blood circuit (selected from those shown in FIG. 18A or B, each circuit embodiment was tested). The BRECS-d unit and the circuit were chosen for each study depending upon which BRECS-d device and which circuit was available on the day of the study, and the data collected from all pairings of BRECS-d units and circuits were combined. In the non-treated sham group, acellular BRECS-d units were used. In the BRECS-d treated group, PPREC-seeded BRECS-d units were employed. Continuous volume resuscitation with normal saline was employed to provide a net balance of 150 mL/h to maintain MAP and cardiac output. The time to death in this porcine model of bacteremic septic shock was 10-16 hours.

Cardiovascular parameters were measured and samples for biochemical analysis were obtained at various time intervals during the experiment. PPREC-seeded BRECS-d treated animals showed overall improvement in cardiovascular parameters, with a 27% improvement in cardiac output. Further, BRECS-d therapy increased average survival time from 6.7 hours in the non-treated sham group to 11.3 hours in the BRECS-d treated group. (Of note, 3 BRECS-d treated animals were terminated at the predetermined 12 hour end point rather than expiring due to sepsis).

### Future Evaluation

Experiments are underway to confirm beneficial time frames for application of frozen and thawed BRECS-d devices. Preliminary studies will initially place a thawed unit immediately on the pig. The time to application also will be varied. Evaluation of these devices will confirm beneficial lag times before placement and use in clinical settings.

### Conclusions

The peritoneal dialysis treatment circuit of FIG. 55 provided acceptable uremic control while maintaining cell viability and functionality in HREC-seeded BRECS-d devices in a sheep model of uremia. The 24-hour CFPD model can be applied as a platform for various types of cell therapy and to PD-based wearable bioartificial kidneys. The circuit of FIG. 55 used in this example allows for potential cell cytokine production and metabolic components to be delivered to an animal with no interference or binding within a second hemofilter. Incorporation of a BRECS-d device into an animal model of septic shock expands therapeutic application to acute kidney injury as well. This example demonstrates a device, readily adaptable to the clinic, that expands function beyond xenographic applications.

### Example 8

### Durability, Freezing and Thawing of Cell-Seeded BRECS-d Devices

This example describes the durability of PPREC- and HREC-seeded BRECS-d units of FIGS. 13-16. Further, cryopreservation and reconstitution of a BRECS-d device of FIGS. 15 and 16 was tested.

### Durability of Cell-Seeded BREC-d Units as Tested In Vitro

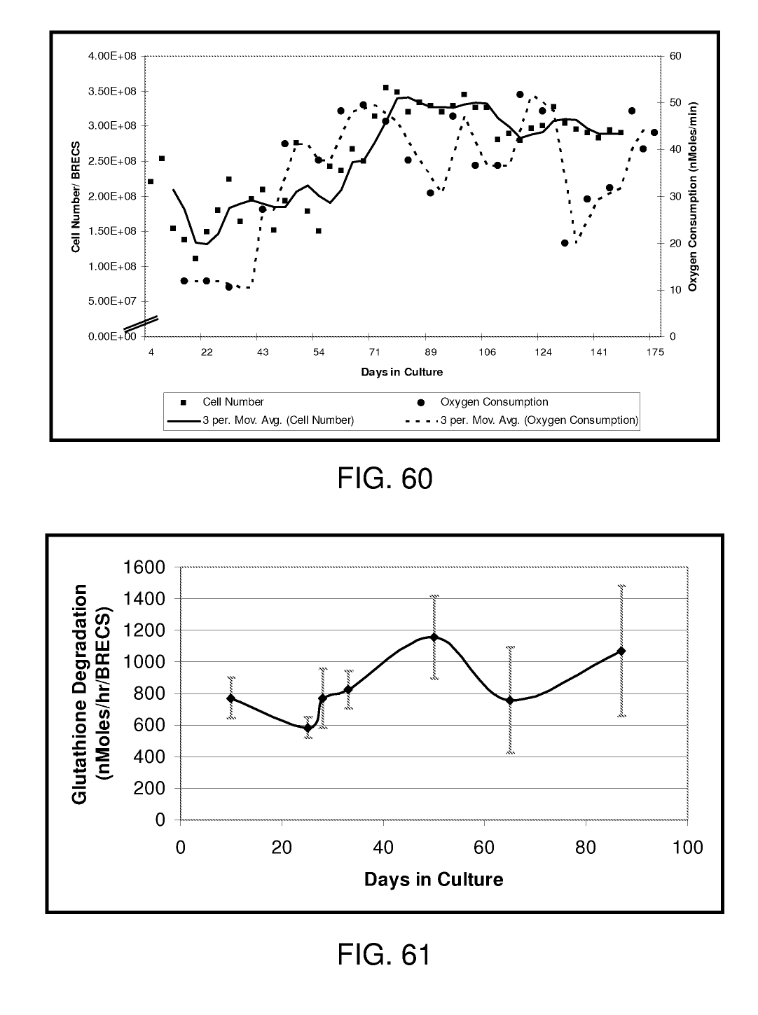

Long term in vitro culture studies were conducted to determine the durability of PPREC- and HREC-seeded cell devices. Cell longevity and functionality in BRECS-d devices are relevant to their targeted chronic therapeutic applications. As such, the viability and metabolic activity of the cells in BRECS-d units were assessed. FIG. 60 shows the results for PPREC-seeded BRECS-d units maintained for more than 6 months in culture. As in previous examples, lactate production was measured to extrapolate cell number and oxygen consumption was measured to monitor metabolic activity and to assess cell viability. In this experiment, BRECS-d devices (as shown in FIGS. 13-16) were tested. Briefly, each BRECS-d device consisted of 20 trabeculated carbon disks seeded with upwards of 10⁸ PPRECs. The disks were coated with collagen IV using the double-sided centrifugation coating technique (as described in Example 6), and were seeded with cells using the double-sided static seeding technique (as described in Example 6). Culture in each BRECS-d unit was performed by perfusing UltraMDCK media through the unit at a rate of 50 mL/min, which provided nutrients and oxygen for maintaining cell viability. Over the course of 186 days in culture, the number of cells in the BRECS-d devices increased, and each device maintained up to 3.5×10⁸ cells. In addition, cells in the tested BRECS-d devices remained viable as demonstrated by a mean oxygen consumption rate of 33 nMoles/min over the course of the study. These results demonstrate the efficiency of BRECS-d devices for both the expansion of cells seeded on the disks and for maintenance of cell viability during the culture period.

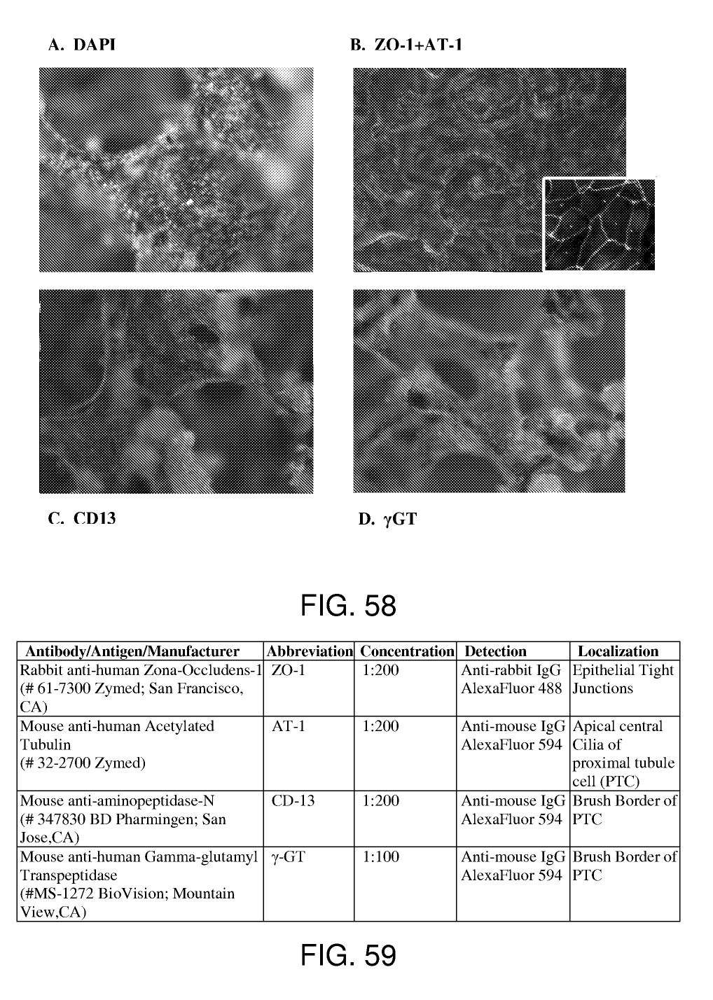

To assess whether cells cultured in BRECS-d devices maintained differentiated renal characteristics over time, HREC-seeded BRECS-d units (as shown in FIGS. 13-16) were analyzed by immunohistochemistry (IHC). The HREC-seeded BRECS-d units were generated by a combination of the double-sided centrifugation collagen IV coating technique and the double-sided static cell seeding technique as previously described. Each BRECS-d unit was perfused with UltraMDCK media, running at a perfusion rate of about 25 mL/min to about 50 mL/min. Commercially available human-specific antibodies and fluorescent conjugates allowed for the assessment of various renal differentiation markers. FIG. 58 shows representative images of HRECs seeded on collagen-IV coated carbon disks after BRECS-d culture of up to 90 days. In this experiment, HREC-seeded disks were removed from BRECS-d units after 90 days in culture and rinsed three times with sterile PBS. The cells were fixed in cold, freshly made 4% paraformaldehyde for 20 minutes on ice, permeated with 0.1% TritonX-100 for 10 minutes on ice, and subsequently blocked for 1 hour using 5% goat serum (# G9023 Sigma-Aldrich) and 1% NaN₃ in PBS. Following the blocking incubation, disks were washed three times with PBS, and incubated overnight at 4° C. with primary antibodies. After incubation, the primary antibodies were removed, followed by a 30 minute dark exposure to fluorescent secondary antibodies and to DAPI staininig. Three blocking buffer washes were performed (two minutes each wash), which was followed by extensive PBS washing prior to visualization. FIG. 59 shows the primary (first column of the table) and secondary (fourth column of the table) antibodies used for the IHC studies. Samples were viewed under fluorescent optics using a Zeiss Axiovert 200 fluorescent microscope.

Results from these studies indicate that HRECs cultured in BRECS-d devices maintained differentiated renal characteristics for up to 90 days. As shown in FIG. 58A, staining by DAPI showed uniform distribution of the nuclei over the entire trabeculated disk surface, indicating even distribution of the cells throughout the carbon disk. Characteristics associated with polarized renal epithelium were evident in the seeded HRECs by the punctate AT-1 positive staining indicative of centralized cilia and the ZO-1 positive staining indicative of epithelial tight junctions (FIG. 58B). Data also suggests that HRECs in BRECS-d devices retained their renal functional characteristics. Prominent CD13 distribution indicated the presence of the brush border enzyme for Na⁺²-dependent amino acid transport (FIG. 58C), and γ-glutamyltranspeptidase (γGT) staining indicated the presence of the brush border enzyme that facilitates glutathione (GSH) metabolism (FIG. 58D).

As an additional test of the functionality and differentiated state of HRECs maintained in BRECS-d units of FIGS. 13-16, γGT activity was assessed. γGT, which is expressed on the brush border membrane of the proximal tubule cells in the kidney, initiates the cleavage of extracellular glutathione (GSH) into its constituent amino acids, which are then transported into cells for maintenance of glutathione homeostasis. A non-destructive, metabolic living assay was established in which the degradation rate of an exogenous supplement of GSH added to a BRECS-d unit was monitored over a 60 minute period, during which time the BRECS-d unit was perfused with 70 mL of recirculating media. Media samples (1 mL) were taken from the BRECS-d chamber at 0 and 60 minutes, and GSH levels were measured. GSH degradation, which is associated with GSH uptake by the HRECs in a BRECS-d unit, was calculated by determining the difference in GSH levels at the two time points during the 1 hour test period. FIG. 61 shows stable GSH degradation rates in BRECS-d units over a 90 day time course in culture (n=4), which is indicative of sustained γGT differentiated renal functions. This data confirms that HRECs in BRECS-d devices retained their renal functions during long term culture.

### Freezing and Thawing of Acellular Devices

As a valuable component to clinical application of a cell therapy device, cryopreservation of BRECS-d units and storage options were evaluated. Liquid nitrogen vapor phase cryopreservation was assessed to determine material integrity under freezing and thawing conditions. Protocols were evaluated to investigate thermodynamic control in BRECS-d units. In these studies, BRECS-d units, as shown in FIGS. 15 and 16, were tested.

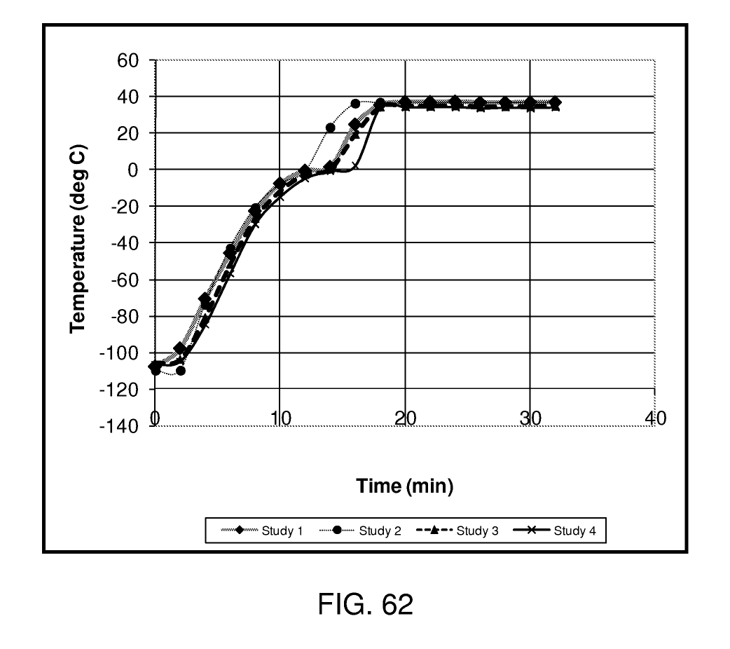

Thawing time is relevant to cell viability and clinical implementation. The initial thaw time for an entire BRECS-d unit was over 45 minutes, and sometimes lasted up to 1.5 hours. To further establish a thawing protocol, twenty six trials were conducted to develop the protocol described below. Each trial involved a single acellular device of FIGS. 15 and 16 being frozen and thawed under varying conditions. Thereafter, four trials were conducted using the developed protocol and the BRECS-d device of FIGS. 15 and 16. In these trials, BRECS-d units were filled with water and a one way valve was placed at the entrance port of each device to allow for volumetric expansion. Alternatively, sealing both the entrance and exit ports with luer caps was determined to be sufficient for acellular freezing. The units were frozen by incremental decreases in temperature from 4° C. to −140° C. over a period of four hours. After varying lengths of time in liquid nitrogen, the units were removed and immediately placed in 90° C. water bath for 12 minutes. This initial thaw period warmed the polycarbonate housing and approximately 20% of the outer media, leaving the disks, baffles, and scaffolds in a frozen cube within the center of each unit. A single pass perfusion circuit was built, which consisted of a media source bottle that was passed through each BRECS-d unit via a pump and tubing system into a collection receptical waste bottle. The single pass perfusion circuit, running at a perfusion rate of 50 mL/min, was initiated with 37° C. media flowing around the frozen cube. At the same time, the units were placed in a 37° C. water bath within an incubator to thaw each system from the outside. To ensure that the temperature of the media in the center of each device would not exceed 39° C., temperature measurements were collected every two minutes using thermocouples (TC) positioned at various locations throughout each BRECS-d unit. FIG. 62 summarizes the four studies, which were performed using the established protocol. As shown, the developed protocol produced a thawed BRECS-d device in 20 minutes while maintaining an internal temperature of no more than 39° C. within the device.

### Cryopreservation and Reconstitution of Cell-Seeded Devices

PPREC-seeded BRECS-d units (shown in FIGS. 15 and 16) were cryopreserved to assess cell retention rate and cell viability parameters after thawing. In brief, each BRECS-d unit was initially flushed with 60 mL of sterile phosphate buffered saline (PBS) and then filled with HTS-Purge Solution (BioLife Solutions, Inc.), a hypothermic solution that helps to prepare cells for cryopreservation. Subsequently, the cryopreservation buffer CryoStor CS5, which contains 5% DMSO, was gradually added to each BRECS-d unit. The entrance port injection site was replaced with a one way outward valve. The temperature drop in each unit was conducted slowly by placement in a Styrofoam cooler and was adjusted from 4° C., to −20° C., to −80° C., and then to the vapor phase of liquid nitrogen (−140° C.) over a period of four hours. The units were then stored in the liquid nitrogen vapor phase for varying lengths of time.

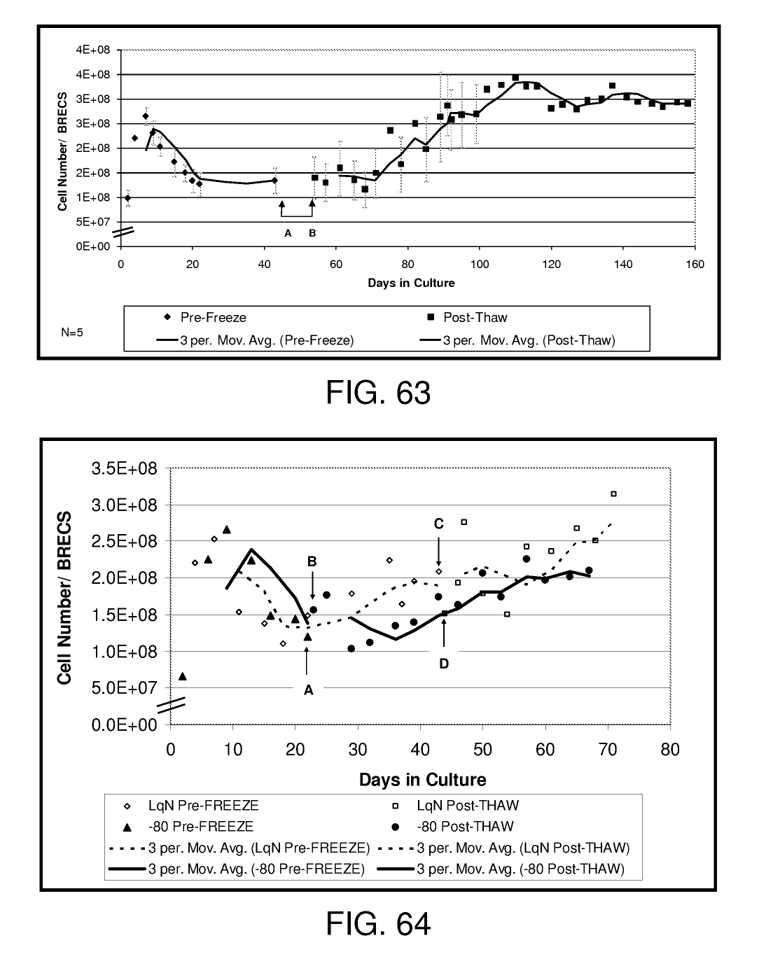

PPREC-seeded BRECS-d units were thawed according to the above-described protocol for acellular devices, resulting in a transition from −140° C. in the liquid nitrogen vapor phase to a 37° C. operating temperature within 20 minutes. The central core of each BRECS-d unit, which contained the cell-seeded disks, remained frozen during the initial 12 minute incubation at 90° C. The cells were subsequently thawed by perfusion with 37° C. media, which rapidly replaced the cryopreservation buffer. FIG. 63 shows the average number of PPRECs in the BRECS-d units before and after the freeze/thaw process (n=5). By comparing the number of cells at the time of cryopreservation and immediately after thawing, it was determined that, on average, 95% of PPRECs were retained in each BRECS-d unit after thawing. BRECS-d units have been cryopreserved in liquid nitrogen vapor phase for up to four months with successful reconstitution. As shown in FIG. 63, cells in post-thaw BRECS-d units appeared to have comparable or even better viability than cells in pre-thaw BRECS-d units as indicated by the increasing number of cells in culture after thawing.

Many clinical facilities have −80° C. freezers but are less likely to possess on-site liquid nitrogen storage. Therefore, additional testing was conducted to confirm that BRECS-d units (shown in FIGS. 15 and 16) could be stored for at least one month at −80° C., after initial liquid nitrogen vapor phase cryopreservation, without negative impact on cell retention or metabolic activity. After two weeks of liquid nitrogen vapor phase storage, two BRECS-d units were transferred to −80° C. storage for analysis at either 1 week or 1 month after transfer. FIG. 64 compares the average cell number in a first group of BRECS-d units stored in liquid nitrogen vapor phase (n=2) with a second group of units that were transferred from liquid nitrogen storage to −80° C. storage for 1 week or 1 month (n=2; one unit was stored at −80° C. for 1 week, and one unit was stored at −80° C. for 1 month). Points A and B indicate cell numbers before freezing and after thawing, respectively, for units transferred from liquid nitrogen storage to −80° C. storage. Points C and D show the cell numbers before freezing and after thawing, respectively, for units stored only in liquid nitrogen vapor phase. As shown, the two experimental groups exhibited comparable post-thaw cell retention rates. This was determined by comparing the number of cells at point A versus point B for cells stored in −80° C., and the number of cells at point C versus point D for cells stored in liquid nitrogen vapor phase. In addition, cells from both experimental groups exhibited similar growth rates after thawing. Finally, the metabolic activity of the PPRECs in BRECS-d units transferred to −80° C. storage, as measured by oxygen consumption rates, showed an average increase of 21% from pre- to post-cryopreservation within 48 hours post-reconstitution (n=2).

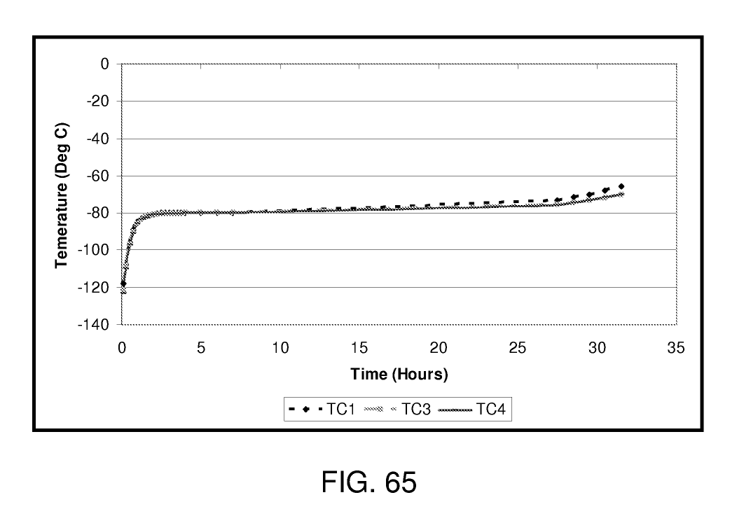

Lastly, studies were done to assess shipment to the clinical site. Dry ice was used to maintain temperature during shipping studies. A study using thermocouples placed inside a cryopreserved acellular BRECS-d unit of FIGS. 15 and 16 indicates that a 36 hour transfer time is feasible as an internal temperature of −80° C. within the shipped device is maintained during that time period (FIG. 65). It is contemplated that use of device insulation and extruded polystyrene foam for a casing is expected to extend these conditions.

### Conclusion

These studies detail the design, fabrication and successful testing of a compact, cryopreservable BRECS-d unit. This innovative, tissue-engineered device provides enabling technology for use in acute and chronic disease applications

### INCORPORATION BY REFERENCE

The entire content of each patent and non-patent document disclosed herein is expressly incorporated herein by reference for all purposes. Moreover, U.S. patent application publication No. 2007/0269489 is expressly incorporated herein by reference for all purposes.

### Other Embodiments and Clinical Objectives

The invention may be embodied in other specific forms without departing from the spirit or essential characteristics thereof. The present embodiments are therefore to be considered in all respects as illustrative and not restrictive.

### APPENDIX I

### Literature Cited

1. Pinsky M R, Vincent J L, Deviere J, Alegre M, Kahn R J, Dupont E: Serum cytokine levels in human septic shock. Chest 1993; 103:565-576.
2. Marty C, Misset B, Tamion F, Fitting C, Carlet J, and Cavallion J M: Circulating interleukin-8 concentrations in patients with multiple organ failure of septic and nonseptic origin. Crit Care Med 1994; 22:673-679.
3. Humes H D, Buffington D A, Lou L, Abrishami S, Wang M, Xia J, and Fissell W H: Cell therapy with a tissue-engineered reduces the multiple-organ consequences of septic shock. Crit Care Med 2003; 31:2421-2428.
4. Humes H D, Fissell W H, Weitzel W F, et al: Metabolic replacement of renal function in uremic animals with a bioartificial kidney containing human cells. Am J Kidney Dis 2002; 39(5):1078-1087.
5. Humes H D, Buffington D A, MacKay S M, et al: Replacement of renal function in uremic animals with a tissue-engineered kidney. Nat Biotechnol 1999; 17:451-455.
6. Humes H D, MacKay S M, Funke A J, et al: Tissue engineering of a bioartificial renal tubule assist device: In vitro transport and metabolic characteristics. Kidney Int 1999; 55:2502-2514.
7. Fissell W H, Dyke D B, Weitzel W F, et al: Bioartificial kidney alters cytokine response and hemodynamics in endotoxin challenged dogs. Blood Purif 2002; 20:55-60.
8. Fissell W H, Lou L, Abrishami S, et al: Bioartificial kidney ameliorates gram-negative bacteria-induced septic shock in uremic animals. J Am Soc Nephrol 2003; 14:454-461.
9. Humes H D, Buffington D A, Lou L, et al: Cell therapy with a tissue-engineered kidney protects against the multi-organ consequences of septic shock. Crit Care Med 2003; 31:2421-2428.
10. Humes H D, Weitzel W F, Bartlett R H, Swaniker F C, Paganini E P, Luderer J R, and Sobota J: Initial clinical results of the bioartificial kidney containing human cells in ICU patients with acute renal failure. Kidney Int 2004; 66:1578-1588.
11. Tumlin J, Wali R, Brennan H D, and Humes H D: Effect of the renal assist device (RAD) on mortality of dialysis-dependent acute renal failure: A randomized, open-labeled, multicenter, Phase II trial [Abstract]. J Am Soc Nephrol 2005; 16:46A.
12. Williams W, Tumlin J, Murray P, Tolwani A, RAD-002 Study Investigators: Renal bioreplacement therapy (RBT) reduces mortality in ICU patients with acute renal failure (ARF). J Am Soc Nephrol 2006; 17:49A.

What is claimed is:

1. An extracorporeal therapeutic system, the system comprising:
a housing defining an interior space;
a frozen substrate having a trabecular structure disposed within the housing; and
at least one cell disposed on the substrate.

2. The system of claim 1, wherein the housing comprises an inlet for receiving a fluid and an outlet for releasing a processed fluid.

3. The system of claim 1, wherein the substrate is initially separate from the housing.

4. The system of claim 1, wherein the substrate is coated with collagen IV.

5. The system of claim 2, wherein at least one flow separator is disposed between the inlet and the substrate.

6. An extracorporeal therapeutic system, the system comprising:
a housing defining an interior space;
a substrate comprising a carbon material coated with niobium disposed within the housing; and
at least one cell disposed on the substrate.

7. The system of claim 6, wherein the substrate has a trabecular structure.

8. The system of claim 6, wherein the substrate is initially separate from the housing.

9. The system of claim 6, wherein the substrate is coated with collagen IV.

10. The system of claim 6, wherein the housing comprises an inlet for receiving a fluid and an outlet for releasing a processed fluid and wherein at least one scaffold to retain the substrate is disposed within the housing.

11. The system of claim 10, wherein at least one flow separator is disposed between the inlet and the substrate.

12. The system of claim 6, wherein the substrate is frozen.

13. A substrate for maintaining cells, the substrate comprising a carbon material coated with niobium and with collagen IV and comprising a trabecular structure.

14. The substrate of claim 13, further comprising a cell.

15. The substrate of claim 14, wherein the cell is a renal cell.

16. The substrate of claim 13, wherein the substrate is frozen.

17. A method for loading an extracorporeal therapeutic system comprising:
thawing a cryopreserved cell; and
loading the cell into the system.

18. The method of claim 17, wherein the system comprises a housing and at least one hollow fiber associated with the housing, and wherein the cryopreserved cell is thawed, mixed with a biomatrix material, and loaded into the hollow fiber.

19. The method of claim 17, wherein the system comprises a housing, and wherein the cryopreserved cell, disposed on a substrate, is loaded into the housing.

20. The method of claim 19, wherein the substrate comprises a carbon material coated with niobium and, optionally, collagen IV.

21. A method for extracorporeal therapy, the method comprising:
connecting an extracorporeal therapeutic system to the peritoneum of a mammal, whereby a peritoneal fluid of the mammal circulates through the system.

22. The method of claim 21, wherein the system comprises a housing and at least one hollow fiber associated with the housing.

23. The method of claim 22, wherein the system further comprises a biomatrix material disposed within the at least one hollow fiber and at least one cell disposed within the biomatrix material.

24. The method of claim 21, wherein the system comprises a housing and a substrate comprising a carbon material coated with niobium and, optionally, collagen IV.

25. The method of claim 21, wherein the system comprises a housing and a frozen substrate having a trabeculated structure.

26. The method of claim 25, wherein the housing comprises an inlet for receiving fluid and an outlet for releasing a processed fluid.

27. The method of claim 25, wherein the system further comprises at least one cell disposed on the substrate.

28. An extracorporeal therapeutic system, the system comprising:
a housing;
at least one hollow fiber associated with the housing;
a biomatrix material disposed within the at least one hollow fiber; and
at least one cell disposed within the biomatrix material.

29. The system of claim 28, wherein the at least one cell is disposed upon a particle.

30. The system of claim 28, wherein the biomatrix material is selected from alginate solution, gelled alginate solution, or physiologic buffer.

31. The system of claim 28, wherein the biomatrix material is selected from nutragen or matrigel.

32. The system of claim 28, wherein the hollow fiber is disposed within the housing.

33. The system of claim 28, wherein the housing comprises an inlet and an outlet.
